(19) United States
(12) Patent Application Publication
(10) Pub. No.: US 2026/0232637 A1
(43) Pub. Date: Aug. 13, 2026

(54) METHODS OF TREATING PERIPHERAL AND CORONARY ARTERY DISEASE

(71) Applicant: VERU INC., Miami, FL (US)

(72) Inventors: Mitchell S. STEINER, Germantown, TN (US); Kester Gary BARNETTE, Denver, NC (US)

(73) Assignee: VERU, INC., Miami, FL (US)

(21) Appl. No.: 19/538,552

(22) Filed: Feb. 12, 2026

Related U.S. Application Data
(60) Provisional application No. 63/757,418, filed on Feb. 12, 2025.

(51) Int. Cl. A61K 31/4178; A61K 31/215; A61K 31/366; A61K 31/40; A61K 31/404; A61K 31/47; A61K 31/505; A61P 9/10

(52) U.S. Cl. CPC A61K 31/4178; A61K 31/215; A61K 31/366; A61K 31/40; A61K 31/404; A61K 31/47; A61K 31/505; A61P 9/10

(57) ABSTRACT

The present invention relates to methods of treating peripheral artery disease or coronary artery disease or other atherosclerotic cardiovascular disease (ASCVD) using colchicine binding-site inhibitor (CBSI) compounds of Formula (I):

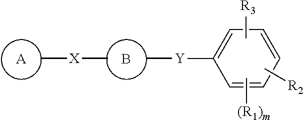

### CROSS REFERENCE TO RELATED APPLICATIONS

This application claims the benefit of priority of U.S. provisional application No. 63/757,418 filed on Feb. 12, 2025, hereby incorporated by reference.

### FIELD OF THE INVENTION

The present invention is directed to methods of treating atherosclerotic cardiovascular diseases (ASCVD) such as peripheral artery disease (PAD) and coronary artery disease (CAD) using colchicine binding site inhibitor (CBSI) compounds of the invention having microtubule-targeting activity (MTA), and formulations including said CBSIs with pharmaceutical acceptable excipients. Unlike colchicine, said anti-atherosclerotic activity with the CBSI compounds of the invention is not a liability when dosed with additional agents that comprise typical pharmacotherapy in these diseases, such as lipid lower agents, anti-platelet agents, other anti-coagulants, anti-hypertensive agents, anti-diabetic agents, anti-inflammatory agents, etc.

### BACKGROUND OF THE INVENTION

Cardiovascular diseases are a major cause of morbidity and mortality worldwide (Cui H et al. Adv Drug Deliv Rev 132:252-269, 2018). An important share of the burden of cardiovascular diseases relates to atherosclerosis, which is a process that is driven by well-known risk factors such as hyperlipidemia, hypertension, diabetes, and inflammation (Abrantes A et al. Circulations Reports 3:457-464, 2021). Inflammation is a key contributor for the initiation and progression of peripheral artery or coronary artery atherosclerosis and acute coronary syndrome (Paul T K et al. Angiology 72:501-502, 2021; Hansson G K et al. N Engl J Med 352:1685-1695, 2005; Libby P et al. J Am Coll Cardiol 54:2129-2138, 2009; Ridker P M et al. Eur Heart J 35:1782-1792, 2014; Wolf D et al. Cir Res 124:315-327, 2019). Atherosclerotic cardiovascular disease (ASCVD) refers to disease of the heart and blood vessels due to the accumulation of plaques. Plaques are lesions in the intima (inner endothelial lining) of arteries that contain a buildup of fats, cholesterol, cellular waste products, calcium, and fibrin. Plaques in and on the artery walls can cause the arteries to harden and/or narrow, blocking blood flow (e.g., stenosis). The plaque also can burst, leading to a blood clot (thrombosis). When ASCVD limits blood flow to the heart, it is known as coronary artery disease (CAD) which can lead to dangerous cardiovascular events such as a heart attack, known scientifically as acute myocardial infarction (AMI). Other conditions that are included within the ASCVD umbrella are: cerebrovascular accident (CVA), transient ischemic attack (TIA), peripheral artery disease (PAD), and abdominal aortic aneurysms (AAA).

Peripheral artery disease (PAD) is a disease characterized by atherosclerosis and/or stenosis of the arteries supplying the limbs. About 20-50% of PAD is asymptomatic. An early and most common symptom of PAD is intermittent claudication that manifests as pain, aching, heaviness, or cramping in the legs that comes from physical exertion, such as walking or climbing stairs, and goes away after rest. In the first five years after diagnosis, 70-80% of patients with claudication have stable symptoms, 10-20% have worsening symptoms, and 1-2% progress to critical limb ischemia (CLI). CLI is a severe form of PAD defined by ischemic rest pain, tissue loss, or gangrene, and is associated with high mortality and morbidity. One in four CLI patients may require amputation or die within one year of diagnosis. In addition to risks associated with their limbs, patients with PAD are also at increased risk for other types of ASCVD such as atherothrombosis of the heart (AMI or acute coronary syndrome (ACS; defined below)), brain (CVA or TIA), renal arteries, mesenteric arteries, etc. The diagnosis of PAD is obtained by measuring ratios of systolic blood pressure at different locations on the limbs. The resting ankle-brachial index (ABI) is considered the first-line test for diagnosing PAD. In this test, a sphygmomanometer cuff and Doppler probe are used to measure the systolic blood pressure in both brachial arteries, as well as the bilateral dorsalis pedis and posterior tibial arteries with the patient in the supine position. The ABI is the ratio of the higher reading of the 2 ankle pressures (dorsalis pedis and posterior tibial) over the higher reading of right or left brachial pressure and should be calculated in each limb. An ABI≤0.90 in either limb is diagnostic of PAD. A recent systematic review estimates that the global prevalence of PAD was 5.6% in 2015, indicating that approximately 236 million adults were living with PAD worldwide. PAD can affect people of any age, but the risk increases with age and is greater in high income countries. In the United States, PAD is estimated to be about 7% incidence, affecting 8.5 million adults and is most common in people 65 or older. There is a slightly elevated risk for blacks of either gender compared to non-Hispanic whites. Lifestyle modifications and medical management can treat asymptomatic to mild PAD. Severe ischemia requires revascularization surgery. Although PAD has long been underappreciated compared with coronary artery disease (CAD) and cerebrovascular disease, PAD is increasingly recognized as an important cause of cardiovascular morbidity and mortality. Although many risk factors are shared between PAD and other forms of atherosclerosis, epidemiological data have increasingly made it clear that PAD warrants recognition as a unique entity.

Since atherosclerosis is a systemic disease process, recent attention has focused on the coincidence of PAD with atherosclerosis in other arterial beds, most commonly CAD and cerebrovascular disease (occasionally including renal artery or mesenteric atherosclerosis). This framework, termed polyvascular disease, is defined by the presence of atherosclerosis in ≥2 arterial beds.

According to the Centers for Disease Control and Prevention (2023), 20.5 million U.S. adults have coronary artery disease (CAD)[also called ischemic heart disease or coronary heart disease], making it the most common type of heart disease in the United States. Atherosclerotic CAD remains the leading cause of mortality worldwide (Collaborators, G.B.D.C.o.D. Lancet 392(10159):1736-88, 2018; Verghese D et al. Am Heart J 277:20-26, 2024; Boland J et al. Current Cardiology Reports 23:6, 2021; Vaduganathan M et al. J Am Coll Cardiol 80:2361-71, 2022). Inflammation and high cholesterol jointly contribute to atherosclerotic disease. It appears that the pathogenesis and progression of CAD, however, is largely driven by inflammation in response to atheromatous plaques containing cholesterol in the arterial wall (Ross R. Am Heart J 138: 5419-420, 1999). In fact, inflammation mediates all stages of atherosclerotic coronary vascular disease: plaque initiation, plaque progression, and plaque rupture and the resulting thrombotic complications (Aldana-Bitar J et al. Progress in Cardiovascular Diseases 84:68-75, 2024). Even with cholesterol reduction therapies, there remains a major and largely untreated residual inflammatory risk. Using high sensitivity C-reactive protein (CRP) blood levels to assess the contribution of inflammation, a recent analysis of 31,245 patients receiving statin lipid-lowering therapy showed that inflammation was a stronger predictor for risk of future major cardiovascular events (MACE) and death than cholesterol by LDL-C(Ridker P M et al. Lancet 401:1293-1301, 2023). The realization that the combined use of aggressive lipid-lowering (statins, etc.; discussed herein) and inflammation-inhibiting therapies might be needed to further reduce atherosclerotic risk has sparked the search for anti-inflammatory medications that could lower the risk of atherosclerotic events in patients with PAD or CAD (Verghese D et al. Am Heart J 277:20-26, 2024; Ridker P M et al. Lancet 401:1293-1301, 2023).

An old drug, colchicine, inhibits tubulin polymerization to disrupt microtubules resulting in broad anti-inflammatory activity (Buckley L F et al. Arterioscler Thromb Vas Biol 44:1031-1041, 2024). Recent randomized controlled trials assessing the role of low-dose colchicine to treat inflammation to reduce MACE, including COLCOT (Efficacy and Safety of Low-Dose Colchicine after Myocardial Infarction), LoDoCo (Low-dose colchicine for secondary prevention of cardiovascular disease) and LoDoCo-2 (Colchicine in Patients with Chronic Coronary Disease) trials, had promising results demonstrating significant cardiovascular risk reduction. Colchicine lowered MACE by 31% among those with stable CAD and by 23% in patients following a recent myocardial infarction (Tardif J C et al. N Engl J Med 381:2497-2505, 2019; Nidorf S M et al. Am Coll Cardiol 61:404-410, 2013; Nidorf S M et al. N Engl J Med 383:1838-1347, 2020). This magnitude of benefit is greater than what has been observed in contemporary trials of lipid lowering medications including those with proprotein convertase subtilisin/kexin type 9 (PCSK-9; discussed herein) inhibitors (Sabatine M S et al. N Engl J Med 376:1713-1722, 2017; Schwartz G G et al. N Engl J Med 379:2097-2107, 2018).

To gain mechanistic insight, Vaidya et al. (Vaidya K et al. JACC: Cardiovascular Imaging 11:305-316, 2018) conducted a prospective open label single center clinical study to evaluate the effects of colchicine on modifying the atheromatous coronary artery plaques by coronary CT angiography (CCTA) imaging in eighty patients who had recent acute coronary syndrome (ACS). ACS is a medical emergency that is a more severe manifestation of CAD. Although CAD can be asymptomatic, ACS is usually symptomatic and can include conditions like unstable angina (USA) and myocardial infarction (MI), and symptoms such as chest pain or pressure, shortness of breath, excessive sweating, dizziness, and fatigue. Patients in these trials received either low dose colchicine plus optimal medical therapy or optimal medical therapy alone for 12 months. The primary endpoint was low attenuation plaque volume (LAPV), a marker of plaque instability on CCTA and a strong predictor of MACE. High sensitivity CRP blood levels, a biomarker of inflammation, were also measured. The study results showed that colchicine+optimal medical therapy versus optimal medical therapy alone significantly reduced the primary endpoint of LAPV by −40.9% and high sensitivity CRP by −37.3% (Vaidya K et al. JACC: Cardiovascular Imaging 11:305-316, 2018). This was an important milestone for the treatment of CAD as colchicine therapy was able to directly modify coronary arterial plaque independent of high dose statins that lower LDL. Further, because of the reduction of high sensitivity CRP, the improvements in plaque morphology were most likely driven by colchicine's anti-inflammatory effects (Aldana-Bitar J et al. Progress in Cardiovascular Diseases 84:68-75, 2024).

Data from these trials led the Food and Drug Administration (FDA) in June 2023 to approve colchicine for reducing cardiovascular events in adults with established atherosclerotic cardiovascular disease (ASCVD) or with multiple risk factors for developing it, making colchicine the first anti-inflammatory drug with such an indication (Verghese D et al. Am Heart J 277:20-26, 2024). Furthermore, the American College of Cardiology/American Heart Association (AHA), European Society of Cardiology as well as national guidelines in Canada and South America have endorsed the use of low-dose colchicine (0.5 mg/d orally) in patients with CAD, especially among those with uncontrolled risk factors or recurrent events (i.e., ACS) despite optimal medical therapy (Visseren F et al. Eur Heart J 42:3227-3337, 2021; Ponte-Negretti C I et al. Archivos de Cardiologia de Mexico 92:99-112, 2022; Licordari R et al. J Clin Med 13:1885, 2024).

Unfortunately, colchicine has well known safety concerns. Colchicine has high potential for drug-drug interactions as it is a substrate for CYP3A4 and P-glycoprotein (PGP). Consequently, inhibitors of CYP3A4 and/or PGP produce drug-drug interactions. Commonly used cardiovascular drugs such as diltiazem and verapamil (CYP3A4 inhibitors), ranolazine (PGP inhibitor), and almost all statins (also known as HMG-CoA reductase inhibitors have PK/PD interactions causing rhabdomyolysis) may interact with colchicine resulting in erratic or higher blood levels of colchicine. Colchicine has a narrow therapeutic index (i.e., margin for error between toxic and therapeutic doses is small) and extreme care must be taken to avoid accidental overdoses, which may be fatal. Accordingly, patients receiving regular colchicine therapy require close clinical supervision (Nidorf S M et al. Am Coll Cardiol 61:404-410, 2013) and consequently, colchicine is not commonly used to treat atherosclerotic CAD. Common side effects are gastrointestinal symptoms (diarrhea, vomiting, and abdominal cramping) and myalgia. Blood dyscrasias which may be fatal, neurotoxicity, and rhabdomyolysis (breakdown of muscles (myotoxicity) that causes renal failure) may also rarely occur (LODOCO FDA package insert, 2023). Statins are also myotoxic and PK/PD interactions between colchicine and statins may result in increased risk of rhabdomyolysis, a life-threatening condition, or other symptomatic myopathies such as muscle weakness, pain, fatigue, or shortness of breath.

There is a large unmet medical need to find anti-atherosclerotic compounds with a novel mechanism of action for treating ASCVD that is both an oral and broad anti-inflammatory agent with low liability for toxicity and low liability for drug-drug interactions with current medical therapies typically used in ASCVD. Such an anti-inflammatory agent should not create a liability that is made worse by concomitant use of CYP3A4 and/or PGP inhibitors, and regardless of hepatic and/or renal impairment. Particularly dangerous, despite ever improving pharmacotherapy, is CAD and its acute variants such as ACS. CAD is the leading cause of death and MACE. Data Bridge Market Research analyses suggest that the total global CAD market was valued at $22 billion in 2022 and is expected to reach the value of $40 billion by the year 2030 (databridgemarketresearch/com). If anti-inflammatory drugs are prescribed in combination with lipid lowering drugs for CAD, they should have similar total global market size. The total global market size for lipid lowering drugs was $33 billion in 2023 and is anticipated to be $45.6 billion by 2033 (precedenceresearch.com). PAD may be diagnosed before atherosclerosis of other artery beds, making its treatment with such anti-inflammatory therapy an attractive way to decrease incidence of other types of ASCVD.

Microtubules are cytoskeletal filaments consisting of α- and β-tubulin heterodimers and are involved in a wide range of cellular functions, including shape maintenance, vesicle transport, cell motility, and division. Tubulin is the major structural component of the microtubules and a verified target for a variety of anticancer drugs. Compounds that are able to interfere with microtubule-tubulin equilibrium in cells can be effective in reducing inflammation. Other compounds that interfere with microtubule-tubulin equilibrium in cells, such as colchicine, paclitaxel and vinblastine, are limited by their toxicity and drug-drug interactions, or may not have anti-inflammatory effects or may have pro-inflammatory effects.

Drugs that target the cytoskeleton, especially the microtubule components, are important therapeutic agents for cancer and inflammation. The clinical activity of these compounds is dictated by the location that these compounds bind on the α and β-tubulin heterodimers that compose the microtubule filament. Three major binding sites on α and β-tubulin subunits have been identified as taxanes-, vinca alkaloid-, and colchicine-binding sites. Such drugs are commonly classified into two major categories: microtubule-stabilizing (e.g., taxanes) and microtubule-destabilizing or depolymerizing agents (e.g., vinca alkaloids and colchicine).

Colchicine has a narrow therapeutic index with no clear distinction between nontoxic, toxic, and lethal doses. Metabolically, colchicine is eliminated via P-glycoprotein (PGP; also known as Multi-Drug Resistance 1 (MDR1) protein). Drug-drug interactions are common with CYP3A4 and P-glycoprotein inhibitors which can increase colchicine blood concentrations to toxic levels leading to colchicine poisoning and death. Life-threatening and fatal toxicities have been observed when colchicine is administered with PGP or strong CYP3A4 inhibitors even at approved therapeutic doses. Additional serious toxicities including myelosuppression, disseminated intravascular coagulation, and cell damage in renal, hepatic, circulatory, and central nervous systems have been observed with approved therapeutic doses of colchicine. These observed serious adverse events limit the clinical use of colchicine.

A major problem with taxanes, as with many biologically active natural products, is its lipophilicity and lack of solubility in aqueous systems. This leads to the use of emulsifiers like Cremophor EL and Tween 80 in clinical preparations, which leads to serious hypersensitivity reactions.

The inventions of this application address a novel method of treating ASCVD, and particularly PAD and CAD, using colchicine binding site inhibitor (CBSI) compounds of this invention having cytoskeleton disruptor activity and formulations including the CBSI compounds of this invention with pharmaceutical acceptable excipients and/or co-therapy with drugs that are part of typical pharmacotherapy for ASCVD, CAD, or PAD. The methods also include treating ASCVD, and particularly PAD and CAD, using CBSI compounds of this invention having cytoskeleton disruptor activity and formulations including combining the CBSI compounds of this invention with pharmaceutical acceptable excipients and/or other drugs typically used in ASCVD, CAD or PAD (as discussed herein) including co-therapy with at least one of CYP3A4 inhibitors or PGP inhibitors; including such co-therapy in patients with hepatic or renal impairment.

### SUMMARY OF THE INVENTION

The invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), in a subject in need thereof by administering to the subject a formulation having a therapeutically effective amount of a colchicine binding site inhibitor (CBSI) compound of Formula (I):

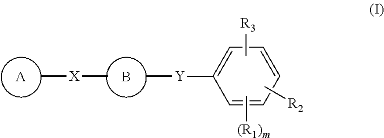

wherein
A is phenyl, indolyl, or indazolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole or benzimidazole, optionally substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O or bond; and
m is 1-3, or a pharmaceutically acceptable salt, hydrate, polymorph, or isomer thereof.

In another embodiment of the invention, the methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD) encompass compounds of Formula I wherein A is indolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; B is an imidazole, optionally substituted with at least one of (C₁-C₄)alkyl;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond;
Y is —C═O; and
m is 1-3, or a pharmaceutically acceptable salt, hydrate, polymorph, or isomer thereof.

Another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD) in a subject in need thereof by administering to the subject a formulation having a therapeutically effective amount of a colchicine binding site inhibitor (CBSI) compound of the Formula VII:

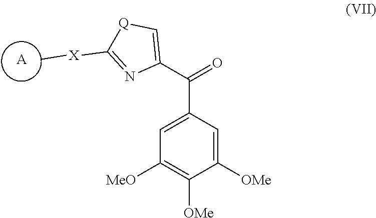

wherein
X is a bond or NH;
Q is NH; and
A is a phenyl, indolyl, or indazolyl ring optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer thereof.

In another embodiment of the invention, the method encompasses compounds of Formula VII wherein X is bond. In yet another embodiment of the invention, the method encompasses compounds of Formula VII, wherein X is a bond; Q is NH; and A is an indolyl ring optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer thereof.

An embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD) in a subject in need thereof by administering to the subject a formulation having a therapeutically effective amount of a colchicine binding site inhibitor (CBSI) compound of the Formula VII(c):

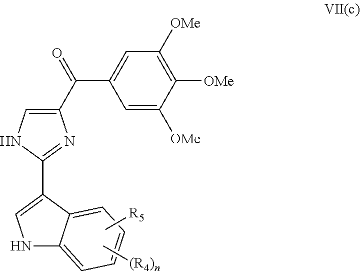

wherein
R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.

Another embodiment of the invention, encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD) in a subject in need thereof by administering to the subject a formulation having a therapeutically effective amount of a colchicine binding site inhibitor (CBSI) compound 17ya represented:

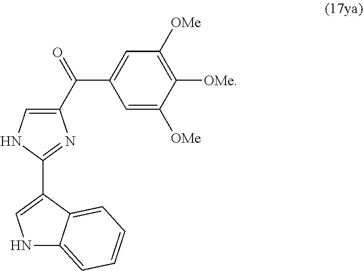 [SMILES: COc1cc(C(=O)c2c[nH]c(-c3c[nH]c4ccccc34)n2)cc(OC)c1OC]

Yet another embodiment of the invention encompasses methods of treating, reducing, inhibiting, slowing the progression of, promoting the regression of, or preventing ASCVD by administering to the subject a formulation having a therapeutically effective amount of a colchicine binding site inhibitor (CBSI) of this invention, wherein the ASCVD is at least one of a cerebrovascular accident (CVA), transient ischemic accident (TIA), or abdominal aortic aneurysm (AAA).

Yet another embodiment of the invention encompasses methods of treating, reducing, inhibiting, slowing the progression of, promoting the regression of, or preventing ASCVD by administering to the subject a formulation having a therapeutically effective amount of a colchicine binding site inhibitor (CBSI) of this invention, wherein the ASCVD is at least one of coronary artery disease (CAD) or peripheral artery disease (PAD).

Yet another embodiment of the invention encompasses methods of treating, reducing, inhibiting, slowing the progression of, promoting the regression of, or preventing CAD by administering to the subject a formulation having a therapeutically effective amount of a colchicine binding site inhibitor (CBSI) of this invention, wherein said CAD is stable CAD.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein said administering of said CBSI compound of this invention treats, reduces, inhibits, slows the progression of, promotes the regression of, or prevents intermittent claudication, critical limb ischemia, or atherothrombosis of the heart, brain or limbs.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein said administering of said CBSI compound of this invention slows the progression or promotes the regression of atheromatous plaques.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein said administering of said CBSI compound of this invention treats, reduces, inhibits, slows the progression of, promotes the regression of, or prevents at least one of plaque initiation, plaque progression, plaque rupture and/or plaque related thrombosis and its complications.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein said administering of said CBSI compound of this invention improves atheromatous plaque morphology or plaque composition.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein said administering of said CBSI compound of this invention improves plaque morphology or plaque composition by reducing plaque volume, decreasing plaque necrotic core size, increasing fibrous cap thickness of the plaque, decreasing the extent of calcification of the plaque, and/or decreasing maladaptive plaque remodeling.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein said administering of said CBSI compound of this invention decreases the low-attenuation plaque volume (LAPV) in said subject measured by coronary computed tomography angiography (CCTA).

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein said administering of said CBSI compound of this invention decreases blood levels of proinflammatory cytokines. In some of these embodiments, the proinflammatory cytokines include one or more of high sensitivity C-reactive protein (CRP), interferon (INF) γ, ferritin, interleukin (IL)-7, IL-15, IL-18, monocyte chemoattractant protein (MCP) 1, macrophage inflammatory protein (MIP)-1α, or MIP-1β.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein said administering of said CBSI compound of this invention decreases both LAPV and CRP.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein said administering of said CBSI compound of this invention treats, inhibits, reduces the incidence of, or prevents adverse cardiovascular events that occur secondary to at least one of said ASCVD, CAD, or PAD.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein said administering of said CBSI compound of this invention treats, reduces, inhibits, slows the progression of, promotes the regression of, or prevents acute coronary syndrome (ACS) that occurs secondary to at least one of said ASCVD, CAD, or PAD.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD) or alternatively acute coronary syndrome (ACS), wherein said administering of said CBSI compound of this invention treats, inhibits, reduces the incidence of, or prevents at least one of acute myocardial infarction (AMI), unstable angina (USA), chest pain or pressure, shortness of breath, excessive sweating, dizziness, or fatigue.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD) or alternatively acute coronary syndrome (ACS), wherein said administering of said CBSI compound of this invention treats, inhibits, reduces the incidence of, or prevents major adverse cardiovascular events (MACE).

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD) or alternatively acute coronary syndrome (ACS), by administering a therapeutically effective amount of said CBSI compound of this invention to treat, reduce, inhibit, slow the progression of, promote the regression of, or prevent major adverse cardiovascular events (MACE), wherein said MACE events include but are not limited to myocardial infarction, stroke, death, heart failure, hospitalization for heart failure, revascularization, e.g., percutaneous coronary intervention (PCI) or coronary artery bypass graft, arrhythmias, endocarditis, aortic dissection, or thrombotic events.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD) or alternatively acute coronary syndrome (ACS), by administering a therapeutically effective amount of said CBSI compound of this invention to treat, inhibit, reduce the incidence of, or prevent MACE despite ongoing ACS or uncontrolled risk factors.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD) or alternatively acute coronary syndrome (ACS), by administering a therapeutically effective amount of said CBSI compound of this invention to treat, reduce, inhibit, slow the progression of, promote the regression of, or prevent major adverse cardiovascular events (MACE), wherein said uncontrolled risk factors include but are not limited to age, diabetes mellitus, hypertension, urologic conditions, chronic kidney disease, smoking, high LDL cholesterol, low HDL cholesterol, elevated triglycerides, family history of CAD, obesity, physical inactivity, or a history of previous cardiovascular events.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein said administering of said CBSI compound of this invention slows the progression of or reverses the decline of physical function.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), by administering a therapeutically effective amount of said CBSI compound of this invention to treat, reduce, inhibit, slow the progression of, promote the regression of, or prevent ASCVD, or CAD, or PAD, wherein said physical function is assessed by six-minute walk test or stress test.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), by administering a therapeutically effective amount of said CBSI compound of this invention to provide the therapeutic benefit of colchicine without the risk of the adverse events associated with colchicine.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), by administering a therapeutically effective amount of said CBSI compound of this invention to treat, reduce, inhibit, slow the progression of, promote the regression of, or prevent ASCVD, or CAD, or PAD, wherein at least one of ASCVD, CAD, or PAD is treated with reduced risk of, or without the risk of, adverse events associated with colchicine use; said adverse events include but not limited to diarrhea, vomiting, abdominal cramps, neutropenia, blood dyscrasias, myalgia, rhabdomyolysis, or neurotoxicity.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein said CBSI compound of this invention is administered in combination with other drugs typically used in ASCVD including CAD and PAD. These other drugs are described herein throughout including in “Pharmacotherapy in ASCVD” and “Pharmaceutical Compositions” sections.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein the other drugs often comprise at least lipid-lowering agents and anti-platelet agents.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein the other drugs may further comprise at least one of anti-coagulants, anti-hypertensives, anti-anginal agents, or anti-diabetics.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein said combination can be administered with reduced risk of, or without the risk of, drug-drug interactions (DDI) between said CBSI compound of this invention and the other drugs used in at least one of ASCVD, CAD, or PAD.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein the other drugs include an HMG-CoA reductase inhibitor (statin). In some embodiments of the invention, said statin is at least one of atorvastatin, fluvastatin, lovastatin, lovastatin extended-release, pitavastatin, pravastatin, rosuvastatin, or simvastatin.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein said CBSI compound of the invention provides the therapeutic benefit of colchicine without the risk of the adverse events associated with colchicine, wherein said CBSI compound is co-administered with at least one of an inhibitor of CYP3A4 or an inhibitor of PGP. In some embodiments of the invention, said CYP3A4 or PGP inhibitor is at least one of a direct acting oral anticoagulant, amiodarone, diltiazem, grapefruit juice, atorvastatin, lovastatin, ranolazine, simvastatin, or verapamil.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein said CBSI compound of the invention provides the therapeutic benefit of colchicine without the risk of the adverse events associated with colchicine, wherein said subject has renal or hepatic impairment. In some embodiments of the invention, said subject with renal or hepatic impairment is administered the CBSI compound of the invention as co-therapy with said at least one of an inhibitor of CYP3A4 or an inhibitor of PGP. In some embodiments of the invention, said inhibitor of CYP3A4 or an inhibitor of PGP is at least one of a direct acting oral anticoagulant, amiodarone, diltiazem, grapefruit juice, atorvastatin, lovastatin, ranolazine, simvastatin, or verapamil.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein said CBSI compound of this invention can be administered with reduced risk of, or without the risk of, drug toxicity despite administration of accepted therapeutic doses, or without need for dose adjustments.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein at least one of said ASCVD, CAD, or PAD can be treated by administering said CBSI compound of this invention with reduced risk of, or without the risk of, said adverse events associated with colchicine, wherein said adverse events include but are not limited to diarrhea, vomiting, abdominal cramps, neutropenia, blood dyscrasias, myalgia, rhabdomyolysis, or neurotoxicity.

Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), by administering a therapeutically effective amount of said CBSI of this invention or co-therapy of said CBSI of this invention plus other drugs used in said subject despite a history of intolerable adverse events due to colchicine therapy in the subject.

An embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein the CBSI compound of the invention is administered in an amount of about 0.1 mg to about 100 mg twice daily.

Another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein the CBSI compound of the invention is administered in an amount of about 1 to about 10 mg twice daily. Another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein the compound of the invention is administered in an amount of about 1.5 mg to about 4.5 mg twice daily.

In yet another embodiment of the methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), in a subject in need thereof by administering to the subject a formulation having a therapeutically effective amount of a colchicine binding site inhibitor (CBSI) compound of the invention further comprising at least one pharmaceutically acceptable excipient.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schedule of expected study evaluations in the CAD and PAD clinical trials (Examples 1 and 2).

### DETAILED DESCRIPTION OF THE INVENTION

Colchicine may be a first-in-class and first FDA approved treatment for the significant indication to treat atherosclerotic inflammation, but unfortunately colchicine has significant safety concerns that may limit its expected widespread use. Compound 17ya is a new molecular entity, small molecule that targets the colchicine binding site on β-tubulin. Like colchicine, compound 17ya inhibits microtubule polymerization and has demonstrated the ability to reduce the most important inflammatory mediators that play a role in the initiation and progression of atherosclerotic CAD. However, because compound 17ya has a different chemical structure than colchicine, it is not a substrate for CYP3A4 and P-glycoprotein potentially eliminating drug-drug interactions concerns associated with colchicine. In contrast to colchicine, compound 17ya has stable pharmacokinetics and low potential for drug-drug interactions; thus, compound 17ya may be administered potentially more safely as a secondary therapy for the reduction of inflammation to slow the progression or promote regression of ASCVD, or specifically CAD or PAD, alone or in combination with statin therapy or other drugs typically administered in ASCVD, CAD or PAD (as discussed herein).

The present invention is directed to anti-atherosclerotic therapy based upon the cytoskeleton disruptor activity of the claimed CBSI compounds that interrupt the intracellular microtubules trafficking network and assembly of inflammasomes. Intended to overcome the disadvantages of the prior art, including but not limited to toxicity and drug-drug interactions, the methods are directed to compounds with specific activity to prevent or reduce atherosclerotic cardiovascular disease (ASCVD). To address the need for novel, rapidly-acting, non-toxic, oral anti-inflammatory compounds as ASCVD therapy, the inventors proposed a method of treating at least one of peripheral artery disease (PAD) or coronary artery disease (CAD) by the administration of the colchicine binding site inhibitor (CBSI) compounds of the invention as described herein throughout and including Formulas I through XXII, and compound 17ya described below.

As discussed above, colchicine is a known tubulin inhibitor that binds to its own binding site on tubulin known as the colchicine binding site. Accordingly, compound 17ya (shown below) and related compounds such as those encompassed by generic Formula I and other sub-genera such as Formulas VII and VII(c), among many others herein below, are known as colchicine binding site inhibitor (CBSI) compounds of the invention. These CBSI compounds have been characterized as possessing therapeutic activity in the treatment of cancer and viral infections, and possessing preclinical anti-inflammatory activity. However, previous to this invention, these CBSI compounds have not been investigated in atherosclerotic cardiovascular diseases (ASCVDs) such as peripheral artery disease (PAD) or coronary artery disease (CAD).

Until now, no one has investigated the properties of the compounds of the invention in ASCVDs to determine whether or not such compounds are therapeutically effective, or even effective at all. Many classes of anti-inflammatory agents have been in existence for many decades. E.g., the steroidal glucocorticoid receptor agents discovered in 1953 as hydrocortisone and cortisone, and their many synthetic derivatives such dexamethasone. Similarly, many nonsteroidal anti-inflammatory drugs (NSAIDs) such as ibuprofen have been in use for decades. The skilled artisan will also be aware of many biologic agents that bind to specific inflammatory mediators that have been used as anti-inflammatory agents. Examples include etanercept (a TNF-α R/Fc fusion protein), adalimumab (an anti-TNF-α mAb), infliximab (an anti-TNF-α mAb), tocilizumab (an anti-IL-6R), golimumab (an anti-TNF agent), abatacept (an anti-CD28 agent), rituximab (an anti-CD20 agent), anakinra (an IL-1 inhibitor), canakinumab (an IL-1 agent), and rilonacept (an IL-1 agent). Though some have been tested in clinical trials, none of these classes of anti-inflammatory agents has ever been approved as anti-inflammatory treatment of ASCVD, CAD or PAD. Further, although many preclinical non-colchicine CBSI agents exist (McLoughlin E C, et al. Pharmaceuticals 13(1):8; 2020), no CBSI agent has ever been approved for any purpose.

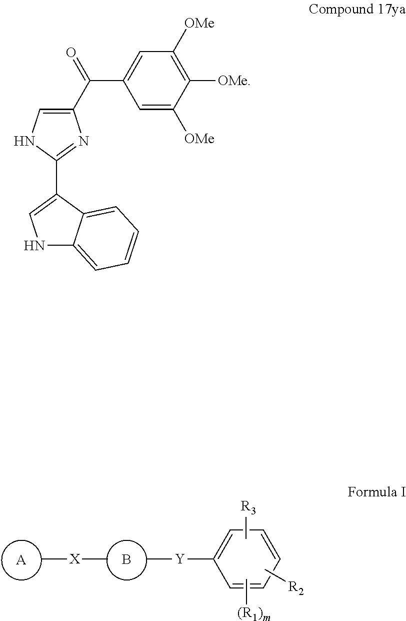

The CBSI compounds of this invention, namely compound 17ya and compounds of Formula I, may provide anti-inflammatory therapy for PAD and CAD, including ACS, similar to colchicine but without the toxicities, drug-drug interactions, and narrow therapeutic index of colchicine that are a liability in this already frail ASCVD patient population. Compound 17ya is evaluated in a small Phase 2 dose finding proof of concept study (40 patients per arm) to assess the progression of coronary atherosclerosis in patients with stable CAD using as the primary endpoint coronary plaque volume and composition measured by coronary CT angiography (CCTA) imaging. More specifically, low attenuation plaque volume (LAPV) has been shown be an excellent predictor of cardiovascular events in the SCOT-HEART Trial (Scottish Computed Tomography of the HEART) (Williams M C et al. Circulation 141:1452-1462, 2020). Furthermore, US FDA has granted drug approvals from CAD atherosclerosis clinical studies using coronary or carotid artery imaging as a primary endpoint to support the indication for the treatment to slow the progression or promote regression of atherosclerotic disease (see, CRESTOR package insert and NIASPAN package insert).

Compound 17ya is evaluated as anti-atherosclerotic and anti-inflammatory treatment for patients suffering with ASCVD. Compound 17ya is an oral novel microtubule disruptor that targets the colchicine binding site of beta tubulin, and binds and crosslinks both the α and β tubulin subunits to inhibit tubulin polymerization and to induce depolymerization of microtubules at low nanomolar concentrations. Compound 17ya is more effective than colchicine in inhibiting tubulin polymerization. Compound 17ya is orally bioavailable with a 5-hour half-life. Unlike colchicine, compound 17ya is not a substrate for P-glycoprotein (PGP) or CYP3A4, or other efflux channels or CYP enzymes. Overall preclinical data from in vitro and in vivo inflammation studies show that compound 17ya treatment suppressed all cytokines and chemokines tested which includes IL-la, IL-10, IL-6, IL-8, TNF-α, Interferon-7, and IP-10 (CXCL-10). In Phase 2 and 3 pulmonary inflammation COVID-19 clinical studies, compound 17ya has demonstrated anti-inflammatory activity. Likewise, in preclinical evaluation of influenza infections. Once the results of Example 1 are published, it will be confirmed whether Compound 17ya can be used as an anti-atherosclerotic agent in CAD or PAD. Furthermore, the results will confirm whether Compound 17ya has an improved therapeutic index or improved drug-drug interaction or improved pharmacokinetic safety profile in this population compared to colchicine.

The safety database consists of 266 dosed patients from the compound 17ya clinical development program comprised of the Phase 2 and Phase 3 studies in hospitalized COVID-19 patients for acute use (149 patients at 9 mg daily for 21 days), as well as data from the Phase 1b/2 (Markowski M C, et al., Clin. Cancer Res 28(13): 2789-2795, 2022) and Phase 3 (abstract in Dreicer et al. J Clin Oncology 40(6)_suppl. https://doi.org/10.1200/JCO.2022.40.6_suppl.TPS217 (VERACITY Phase 3)) studies in prostate cancer for chronic use (117 patients treated at up to 32 mg daily for up to 3 years).

The invention encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of Formula (I):

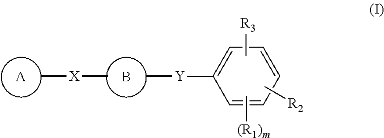

wherein
A is phenyl, indolyl, or indazolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole, thiazole, or benzimidazole, optionally substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond, NH, (C₁-C₄)alkyl, O, or S;
Y is a bond, —C═O, —C═S, SO₂, SO or S; and
m is 1-3, or a pharmaceutically acceptable salt, hydrate, polymorph, or isomer thereof.

Examples of CBSI compounds of Formula I include, but are not limited to, 2-phenyl-4-(3,4,5-trimethoxyphenyl)-1H-benzo[d]imidazole (3), 2-(1H-indol-3-yl)-4-(3,4,5-trimethoxyphenyl)-1H-benzo[d]imidazole (4), 2-(1H-indol-5-yl)-4-(3,4,5-trimethoxyphenyl)-1H-benzo[d]imidazole (5), or 3-(4-(3,4,5-trimethoxyphenyl)-1H-benzo[d]imidazol-2-yl)-1H-indazole (6).

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of Formula (II):

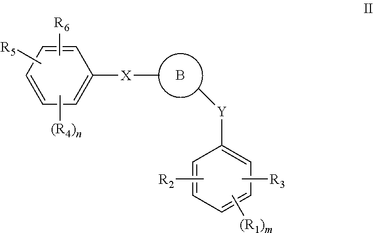

wherein
B is an imidazole, thiazole, or benzimidazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁, R₂, R₃, R₄, R₅ and R₆ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O or bond;
n is 1-3; and
m is 1-3; or a pharmaceutically acceptable salt, hydrate, polymorph, or isomer thereof.

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of Formula (III):

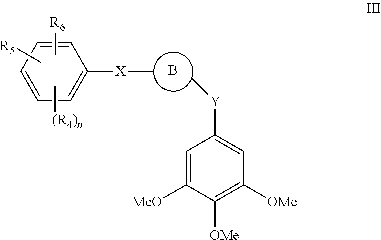

B is an imidazole, thiazole or benzimidazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₄, R₅ and R₆ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O or bond; and
n is 1-3; or a pharmaceutically acceptable salt, hydrate, polymorph, or isomer thereof.

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of Formula (IV):

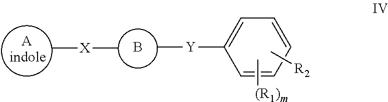

wherein ring A is an indolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole or benzimidazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁ and R₂ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O or bond; and
m is 1-4; or a pharmaceutically acceptable salt, hydrate, polymorph, or isomer thereof.

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of Formula IV(a):

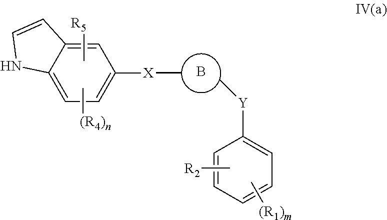

wherein
B is an imidazole or benzimidazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁, R₂, R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
X is a bond or NH;
Y is —C═O or bond;
n is 1-2; and
m is 1-4; or a pharmaceutically acceptable salt, hydrate, polymorph, or isomer thereof.

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of Formula (V):

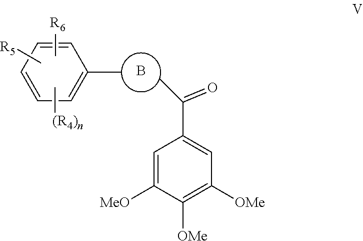

B is an imidazole or thiazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₄, R₅ and R₆ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
n is 1-3; or a pharmaceutically acceptable salt, hydrate, polymorph, or isomer thereof. Examples of CBSI compounds of Formula V include, but are not limited to, (2-phenyl-thiazol-4-yl)-(3,4,5-trimethoxyphenyl)methanone (8f), [2-(3-fluorophenyl)-thiazol-4-yl](3,4,5-trimethoxyphenyl)methanone (8m), (2-(4-fluorophenyl)-thiazol-4-yl)-(3,4,5-trimethoxyphenyl)methanone (8n), (2-(3,4-dimethoxyphenyl)-thiazol-4-yl)-(3,4,5-trimethoxyphenyl)-methanone (8o), (2-(4-nitrophenyl)-thiazol-4-yl)-(3,4,5-trimethoxyphenyl)methanone (8p), 4-(4-(3,4,5-trimethoxybenzoyl)-thiazol-2-yl)-benzonitrile (8q), (2-(4-(trifluoromethyl)-phenyl)-thiazol-4-yl)(3,4,5-trimethoxyphenyl)methanone (8t), (2-(4-bromophenyl)-thiazol-4-yl)-(3,4,5-trimethoxyphenyl)methanone (8u), and (2-(4-ethylphenyl)-thiazol-4-yl)-(3,4,5-trimethoxy-phenyl)methanone (8v).

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula (VI):

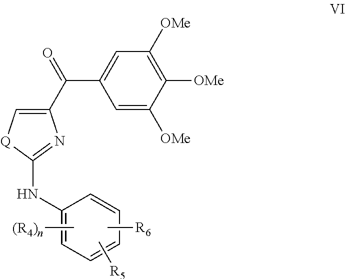

wherein
R₄, R₅ and R₆ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
Q is S or NH; and
n is 1-3; or a pharmaceutically acceptable salt, hydrate, polymorph, or isomer thereof.

Preferably, the variables for the compounds of Formula (VI) for R₄, R₅ and R₆ are independently hydrogen, (C₁-C₄)alkyl, halo(C₁-C₄)alkyl, O—(C₁-C₄)alkyl, O(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; Q is S or NH; and n is 1-3; or a pharmaceutically acceptable salt, hydrate, polymorph, or isomer thereof.

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula VI in the following Table 1:

TABLE 1

| Formula VI | Compound | R₄ | R₅ | R₆ | Q |
|---|---|---|---|---|---|
| 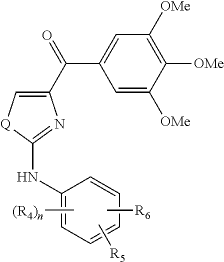 | 5e | H n = 1 | H | H | N |

Examples of CBSI compounds of Formula VI include, but are not limited to, (2-(phenylamino)thiazol-4-yl)(3,4,5-trimethoxyphenyl)methanone (5a), (2-(p-tolylamino)thiazol-4-yl)(3,4,5-trimethoxyphenyl)methanone (5b), (2-(p-fluorophenylamino)thiazol-4-yl)(3,4,5-trimethoxyphenyl)methanone (5c), or (2-((4-chlorophenyl)amino)thiazol-4-yl)(3,4,5-trimethoxyphenyl)methanone (5d).

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula VII:

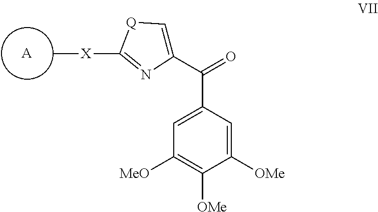

wherein
X is a bond or NH;
Q is S or NH; and
A is a phenyl, indolyl, or indazolyl ring optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer thereof.
   Examples of CBSI compounds of Formula VII include, but are not limited to, (2-(phenylamino)-1H-imidazol-4-yl)(3,4,5-trimethoxyphenyl)methanone (5e), (2-(phenylamino)-1H-imidazol-4-yl)(3,4,5-trimethoxyphenyl)methanone hydrochloride salt (5He), (2-(1H-indol-3-yl)-1H-imidazol-4-yl)(3,4,5-trimethoxyphenyl)methanone (17ya), (2-(phenylamino)thiazol-4-yl)(3,4,5-trimethoxyphenyl)methanone (5a), (2-(p-tolylamino)thiazol-4-yl)(3,4,5-trimethoxyphenyl)methanone (5b), (2-(p-fluorophenylamino)thiazol-4-yl)(3,4,5-trimethoxyphenyl)methanone (5c), and (2-((4-chlorophenyl)amino)thiazol-4-yl)(3,4,5-trimethoxyphenyl)methanone (5d).

Preferably, the variables in the compounds of Formula VII are X is a bond; Q is NH; and A is an indolyl ring optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer thereof.

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula VII(a):

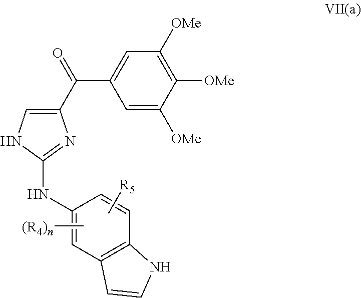

wherein
R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula VII(b):

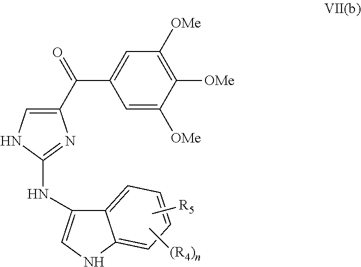

wherein R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula VII(c):

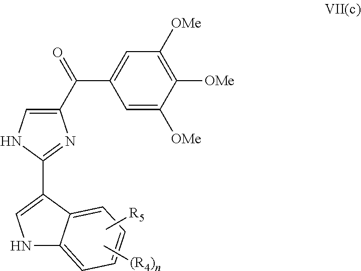

wherein R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.

Examples of CBSI compounds of Formula VII(c) include, but are not limited to, (2-(1H-indol-3-yl)-1H-imidazol-4-yl)(3,4,5-trimethoxyphenyl)methanone (17ya).

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula 17ya:

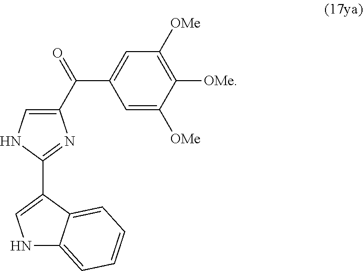 [SMILES: COc1cc(C(=O)c2c[nH]c(-c3c[nH]c4ccccc34)n2)cc(OC)c1OC]

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula XIII:

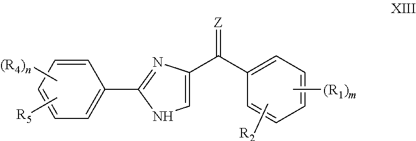

wherein
Z is O;
R₁ and R₄ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₂ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
m is an integer between 1-4; and
n is an integer between 1-4; or a pharmaceutically acceptable salt, hydrate, polymorph, or isomer.

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula XIV:

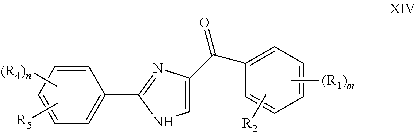

R₁ and R₄ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₂ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
m is an integer between 1-4; and
n is an integer between 1-4;
or a pharmaceutically acceptable salt, hydrate, polymorph, or isomer thereof.

Non limiting examples of CBSI compounds of Formula XIV are selected from: (2-phenyl-1H-imidazol-4-yl)(3,4,5-trimethoxyphenyl)methanone (12aa), (4-fluorophenyl)(2-phenyl-1H-imidazol-4-yl)methanone (12af), (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (4-fluorophenyl)(2-(4-methoxyphenyl)-1H-imidazol-4-yl)methanone (12cb), (2-(p-tolyl)-1H-imidazol-4-yl)(3,4,5-trimethoxyphenyl)methanone (12da), (4-fluorophenyl)(2-(p-tolyl)-1H-imidazol-4-yl)methanone (12db), (4-hydroxy-3,5-dimethoxyphenyl)(2-(p-tolyl)-1H-imidazol-4-yl)methanone (12dc), (2-(4-chlorophenyl)-1H-imidazol-4-yl)(3,4,5-trimethoxyphenyl)methanone (12fa), (2-(4-chlorophenyl)-1H-imidazol-4-yl)(4-fluorophenyl)methanone (12fb), (2-(4-chlorophenyl)-1H-imidazol-4-yl)(4-hydroxy-3,5-dimethoxyphenyl)methanone (12fc), (2-(4-(dimethylamino)phenyl)-1H-imidazol-4-yl)(3,4,5-trimethoxyphenyl)methanone (12ga), (2-(4-(dimethylamino)phenyl)-1H-imidazol-4-yl)(4-fluorophenyl)methanone (12gb), (2-(3,4-dimethoxyphenyl)-1H-imidazol-4-yl)(3,4,5-trimethoxyphenyl)methanone (12ha), (2-(4-(benzyloxy)phenyl)-1H-imidazol-4-yl)(4-fluorophenyl)methanone (12jb), (2-(4-bromophenyl)-1H-imidazol-4-yl)(3,4,5-trimethoxyphenyl)methanone (12la), or (2-(4-(trifluoromethyl)phenyl)-1H-imidazol-4-yl)(3,4,5-trimethoxyphenyl)methanone (12pa).

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula XIVa:

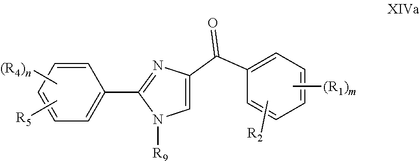

wherein
R₁ and R₄ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₂ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₉ is H, linear or branched alkyl, aryl, CH₂Ph, benzyl, haloalkyl, aminoalkyl, OCH₂Ph, SO₂-Aryl, —(C═O)-Aryl or OH, optionally substituted with at least one of hydrogen, hydroxyl, an aliphatic straight- or branched-chain C₁ to C₁₀ hydrocarbon, alkoxy, haloalkoxy, aryloxy, nitro, cyano, alkyl-CN, halo (e.g., F, Cl, Br, I), haloalkyl, dihaloalkyl, trihaloalkyl, COOH, C(O)Ph, C(O)-alkyl, C(O)O-alkyl, C(O)H, C(O)NH₂, —OC(O)CF₃, OCH₂Ph, amino, aminoalkyl, alkylamino, mesylamino, dialkylamino, arylamino, amido, NHC(O)-alkyl, urea, alkyl-urea, alkylamido (e.g., acetamide), haloalkylamido, arylamido, aryl, and C₅ to C₇ cycloalkyl, arylalkyl, and combinations thereof;
m is an integer between 1-4; and
n is an integer between 1-4;
or a pharmaceutically acceptable salt, hydrate, polymorph, or isomer thereof.

Non limiting examples of CBSI compounds of Formula XIVa are selected from: (4-fluorophenyl)(2-phenyl-1-(phenylsulfonyl)-1H-imidazol-4-yl)methanone (11af), (4-fluorophenyl)(2-(4-methoxyphenyl)-1-(phenylsulfonyl)-1H-imidazol-4-yl)methanone (11cb), (4-fluorophenyl)(1-(phenylsulfonyl)-2-(p-tolyl)-1H-imidazol-4-yl)methanone (11db), (2-(4-chlorophenyl)-1-(phenylsulfonyl)-1H-imidazol-4-yl)(4-fluorophenyl)methanone (11fb), (2-(4-(dimethylamino)phenyl)-1-(phenylsulfonyl)-1H-imidazol-4-yl)(3,4,5-trimethoxyphenyl)methanone (11ga), (2-(4-(dimethylamino)phenyl)-1-(phenylsulfonyl)-1H-imidazol-4-yl)(4-fluorophenyl)methanone (11gb), (2-(3,4-dimethoxyphenyl)-1-(phenylsulfonyl)-1H-imidazol-4-yl)(3,4,5-trimethoxyphenyl)methanone (11ha), (2-(4-(benzyloxy)phenyl)-1-(phenylsulfonyl)-1H-imidazol-4-yl)(4-fluorophenyl)methanone (11jb), (2-(4-(dimethylamino)phenyl)-1-((4-methoxyphenyl)sulfonyl)-1H-imidazol-4-yl)(4-fluorophenyl)methanone (12gba), (1-benzyl-2-(p-tolyl)-1H-imidazol-4-yl)(3,4,5-trimethoxyphenyl)methanone (12daa), (1-methyl-2-(p-tolyl)-1H-imidazol-4-yl)(3,4,5-trimethoxyphenyl)methanone (12dab), or (4-fluorophenyl)(2-(4-methoxyphenyl)-1-methyl-1H-imidazol-4-yl)methanone (12cba).

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula XV:

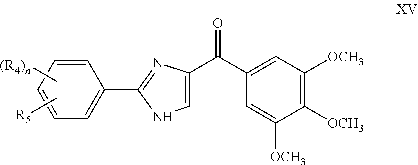

wherein
R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.

Non limiting examples of CBSI compounds of Formula XV are selected from: (2-phenyl-1H-imidazol-4-yl)(3,4,5-trimethoxyphenyl)methanone (12aa), (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (2-(p-tolyl)-1H-imidazol-4-yl)(3,4,5-trimethoxyphenyl)methanone (12da), (3,4,5-trimethoxyphenyl)(2-(3,4,5-trimethoxyphenyl)-1H-imidazol-4-yl)methanone (12ea), (2-(4-chlorophenyl)-1H-imidazol-4-yl)(3,4,5-trimethoxyphenyl)methanone (12fa), (2-(4-(dimethylamino)phenyl)-1H-imidazol-4-yl)(3,4,5-trimethoxyphenyl)methanone (12ga), (2-(3,4-dimethoxyphenyl)-1H-imidazol-4-yl)(3,4,5-trimethoxyphenyl)methanone (12ha), (2-(2-(trifluoromethyl)phenyl)-1H-imidazol-4-yl)(3,4,5-trimethoxyphenyl)methanone (12ia), (2-(4-(benzyloxy)phenyl)-1H-imidazol-4-yl)(3,4,5-trimethoxyphenyl)methanone (12ja), (2-(4-hydroxyphenyl)-1H-imidazol-4-yl)(3,4,5-trimethoxyphenyl)methanone (12ka), (2-(4-bromophenyl)-1H-imidazol-4-yl)(3,4,5-trimethoxyphenyl)methanone (12la), or (2-(4-(trifluoromethyl)phenyl)-1H-imidazol-4-yl)(3,4,5-trimethoxyphenyl)methanone (12pa).

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula XVI:

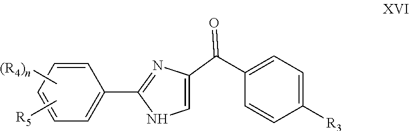

wherein
R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₃ is I, Br, Cl, or F; and
n is 1-4; or a pharmaceutically acceptable salt, hydrate, polymorph, or isomer.

Non limiting examples of CBSI compounds of Formula XVI are selected from: (4-fluorophenyl)(2-phenyl-1H-imidazol-4-yl)methanone (12af), (4-fluorophenyl)(2-(4-methoxyphenyl)-1H-imidazol-4-yl)methanone (12cb), (4-fluorophenyl)(2-(p-tolyl)-1H-imidazol-4-yl)methanone (12db), 4-fluorophenyl)(2-(3,4,5-trimethoxyphenyl)-1H-imidazol-4-yl)methanone (12eb), (2-(4-chlorophenyl)-1H-imidazol-4-yl)(4-fluorophenyl)methanone (12fb), (2-(4-(dimethylamino)phenyl)-1H-imidazol-4-yl)(4-fluorophenyl)methanone (12gb), or (2-(4-(benzyloxy)phenyl)-1H-imidazol-4-yl)(4-fluorophenyl)methanone (12jb).

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula XVII:

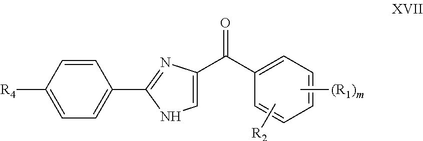

wherein
R₄ is H, O—(C₁-C₄)alkyl, I, Br, Cl, F, (C₁-C₄)alkyl, halo(C₁-C₄)alkyl, amino(C₁-C₄)alkyl, OCH₂Ph, OH, CN, NO₂, —NHCO—(C₁-C₄)alkyl, COOH, C(O)O—(C₁-C₄)alkyl or C(O)H; wherein R₁ and R₂ are independently H, O—(C₁-C₄)alkyl, I, Br, Cl, F, (C₁-C₄)alkyl, halo(C₁-C₄)alkyl, amino(C₁-C₄)alkyl, OCH₂Ph, OH, CN, NO₂, —NHCO—(C₁-C₄)alkyl, COOH, C(O)O—(C₁-C₄)alkyl or C(O)H; and
m is 1-4; or a pharmaceutically acceptable salt, hydrate, polymorph, or isomer thereof.

Non limiting examples of CBSI compounds of Formula XVII are selected from: (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (4-fluorophenyl)(2-(4-methoxyphenyl)-1H-imidazol-4-yl)methanone (12cb), (2-(p-tolyl)-1H-imidazol-4-yl)(3,4,5-trimethoxyphenyl)methanone (12da), (4-fluorophenyl)(2-(p-tolyl)-1H-imidazol-4-yl)methanone (12db), (4-hydroxy-3,5-dimethoxyphenyl)(2-(p-tolyl)-1H-imidazol-4-yl)methanone (12dc), (2-(4-chlorophenyl)-1H-imidazol-4-yl)(3,4,5-trimethoxyphenyl)methanone (12fa), (2-(4-chlorophenyl)-1H-imidazol-4-yl)(4-fluorophenyl)methanone (12fb), (2-(4-chlorophenyl)-1H-imidazol-4-yl)(3,4,5-trihydroxyphenyl)methanone (13fa), (2-(4-(dimethylamino)phenyl)-1H-imidazol-4-yl)(3,4,5-trimethoxyphenyl)methanone (12ga), (2-(4-(dimethylamino)phenyl)-1H-imidazol-4-yl)(4-fluorophenyl)methanone (12gb), (2-(4-(benzyloxy)phenyl)-1H-imidazol-4-yl)(4-fluorophenyl)methanone (12jb), (2-(4-hydroxyphenyl)-1H-imidazol-4-yl)(3,4,5-trimethoxyphenyl)methanone (12ka), (2-(4-bromophenyl)-1H-imidazol-4-yl)(3,4,5-trimethoxyphenyl)methanone (12la), or (2-(4-(trifluoromethyl)phenyl)-1H-imidazol-4-yl)(3,4,5-trimethoxyphenyl)methanone (12pa).

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula XVII represented by the structure of formula 12fb:

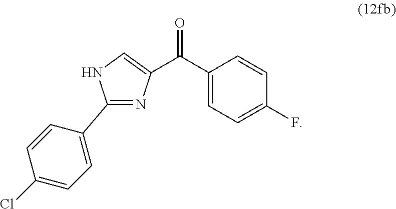 [SMILES: O=C(c1ccc(F)cc1)c1c[nH]c(-c2ccc(Cl)cc2)n1]

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula XVII represented by the structure of formula 12cb:

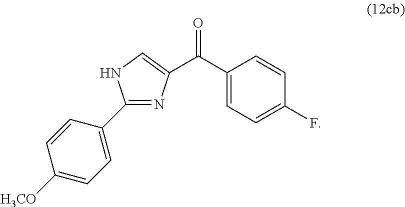 [SMILES: COc1ccc(-c2nc(C(=O)c3ccc(F)cc3)c[nH]2)cc1]

Non limiting examples of CBSI compounds of this invention are selected from: (4-methoxyphenyl)(2-phenyl-1H-imidazol-1-yl)methanone (12aba), (2-phenyl-1H-imidazol-1-yl)(3,4,5-trimethoxyphenyl)methanone (12aaa), 2-phenyl-1-(phenylsulfonyl)-1H-imidazole (10a), 2-(4-nitrophenyl)-1-(phenylsulfonyl)-1H-imidazole (10x), 2-(4-(benzyloxy)phenyl)-1-(phenylsulfonyl)-1H-imidazole (10j), or 2-(2H-indol-5-yl)-4-(3,4,5-trimethoxyphenyl)-1H-imidazo[4,5-c]pyridine (7).

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula XIX:

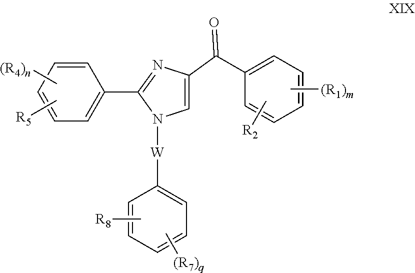

wherein
W is C═O, C═S, SO₂, or S═O;
R₁, R₄ and R₇ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₂, R₅ and R₈ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
m is 1-4;
n is 1-4; and
q is 1-4;
or its pharmaceutically acceptable salt, hydrate, polymorph, or isomer.

Non limiting examples of CBSI compounds of Formula XIX are selected from: (2-(4-(dimethylamino)phenyl)-1-((4-methoxyphenyl)sulfonyl)-1H-imidazol-4-yl)(3,4,5-trimethoxyphenyl)methanone (11gaa); (2-(4-bromophenyl)-1-(phenylsulfonyl)-1H-imidazol-4-yl)(3,4,5-trimethoxyphenyl)methanone (11a), (4-fluorophenyl)(2-(4-methoxyphenyl)-1-(phenylsulfonyl)-1H-imidazol-4-yl)methanone (11cb), (2-(4-chlorophenyl)-1-(phenylsulfonyl)-1H-imidazol-4-yl)(4-fluorophenyl)methanone (11fb), (4-fluorophenyl)(2-phenyl-1-(phenylsulfonyl)-1H-imidazol-4-yl)methanone (11af), (4-fluorophenyl)(1-(phenylsulfonyl)-2-(p-tolyl)-1H-imidazol-4-yl)methanone (11db), (2-(4-(dimethylamino)phenyl)-1-(phenylsulfonyl)-1H-imidazol-4-yl)(3,4,5-trimethoxyphenyl)methanone (11ga), (2-(4-(dimethylamino)phenyl)-1-(phenylsulfonyl)-1H-imidazol-4-yl)(4-fluorophenyl)methanone (11gb), (2-(3,4-dimethoxyphenyl)-1-(phenylsulfonyl)-1H-imidazol-4-yl)(3,4,5-trimethoxyphenyl)methanone (11ha), (2-(4-(benzyloxy)phenyl)-1-(phenylsulfonyl)-1H-imidazol-4-yl)(4-fluorophenyl)methanone (11jb), or (2-(4-(dimethylamino)phenyl)-1-((4-methoxyphenyl)sulfonyl)-1H-imidazol-4-yl)(4-fluorophenyl)methanone (12gba).

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula XIX represented by the structure of formula 11cb:

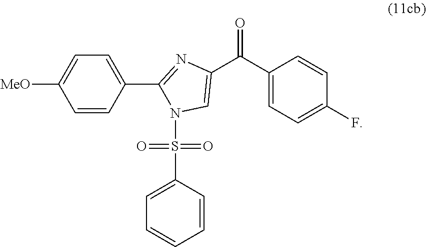 [SMILES: COc1ccc(-c2nc(C(=O)c3ccc(F)cc3)cn2S(=O)(=O)c2ccccc2)cc1]

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula XIX represented by the structure of formula 11fb:

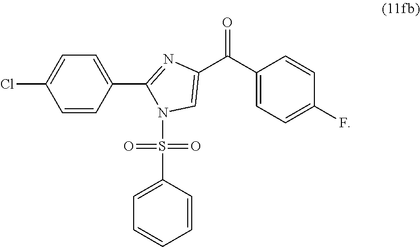 [SMILES: O=C(c1ccc(F)cc1)c1cn(S(=O)(=O)c2ccccc2)c(-c2ccc(Cl)cc2)n1]

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula XX:

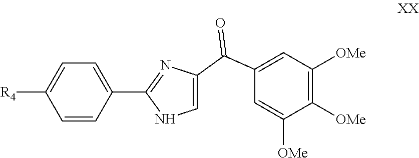

wherein
R₄ is independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer.

Non limiting examples of CBSI compounds of Formula XX are selected from: (2-phenyl-1H-imidazol-4-yl)(3,4,5-trimethoxyphenyl)methanone (12aa), (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (2-(p-tolyl)-1H-imidazol-4-yl)(3,4,5-trimethoxyphenyl)methanone (12da), (2-(4-chlorophenyl)-1H-imidazol-4-yl)(3,4,5-trimethoxyphenyl)methanone (12fa), (2-(4-(dimethylamino)phenyl)-1H-imidazol-4-yl)(3,4,5-trimethoxyphenyl)methanone (12ga), (2-(2-(trifluoromethyl)phenyl)-1H-imidazol-4-yl)(3,4,5-trimethoxyphenyl)methanone (12ia), (2-(4-(benzyloxy)phenyl)-1H-imidazol-4-yl)(3,4,5-trimethoxyphenyl)methanone (12ja), (2-(4-hydroxyphenyl)-1H-imidazol-4-yl)(3,4,5-trimethoxyphenyl)methanone (12ka), (2-(4-bromophenyl)-1H-imidazol-4-yl)(3,4,5-trimethoxyphenyl)methanone (12la), or (2-(4-(trifluoromethyl)phenyl)-1H-imidazol-4-yl)(3,4,5-trimethoxyphenyl)methanone (12pa).

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula XX represented by the structure of formula 12da:

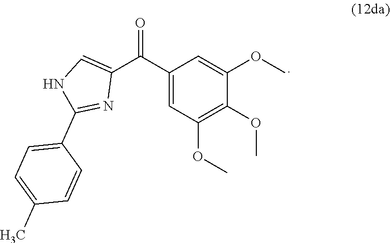 [SMILES: COc1cc(C(=O)c2c[nH]c(-c3ccc(C)cc3)n2)cc(OC)c1OC]

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula XX represented by the structure of formula 12fa:

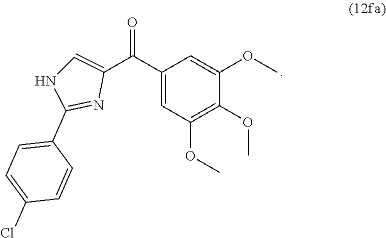 [SMILES: COc1cc(C(=O)c2c[nH]c(-c3ccc(Cl)cc3)n2)cc(OC)c1OC]

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula XXI:

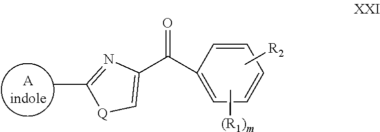

wherein
A is indolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
Q is S or NH;
R₁ and R₂ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, N1₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
m is 1-4; or a pharmaceutically acceptable salt, hydrate, polymorph, or isomer thereof.

In one embodiment of the method of treating, reducing, inhibiting, slowing the progression of, promoting the regression of, or preventing atherosclerotic cardiovascular disease (ASCVD), A ring of compound of Formula XXI is substituted 5-indolyl. In another embodiment the substitution is —(C═O)-Aryl. In another embodiment, the aryl is 3,4,5-(OCH₃)₃-Ph. In another embodiment, A ring of compound of Formula XXI is 3-indolyl. In another embodiment, A ring of compound of Formula XXI is 5-indolyl. In another embodiment, A ring of compound of Formula XXI is 2-indolyl. Non limiting examples of CBSI compounds of Formula XXI are selected from: (5-(4-(3,4,5-trimethoxybenzoyl)-1H-imidazol-2-yl)-1H-indol-2-yl)(3,4,5-trimethoxyphenyl)methanone (15xaa); (1-(phenylsulfonyl)-2-(1-(phenylsulfonyl)-2-(3,4,5-trimethoxybenzoyl)-1H-indol-5-yl)-1H-imidazol-4-yl)(3,4,5-trimethoxyphenyl)methanone (16xaa); (2-(1H-indol-3-yl)-1H-imidazol-4-yl)(3,4,5-trimethoxyphenyl)methanone (17ya); (2-(1H-indol-2-yl)thiazol-4-yl)(3,4,5-trimethoxyphenyl)methanone (62a); or (2-(1H-indol-5-yl)thiazol-4-yl)(3,4,5-trimethoxyphenyl)methanone (66a).

A particularly preferred method of treating, reducing, inhibiting, slowing the progression of, promoting the regression of, or preventing atherosclerotic cardiovascular disease (ASCVD) of the invention uses at least one CBSI compound of Formula XXI including (2-(1H-indol-1-yl)-1H-imidazol-4-yl)(3,4,5-trimethoxyphenyl)methanone; (2-(1H-indol-2-yl)-1H-imidazol-4-yl)(3,4,5-trimethoxyphenyl)methanone; (2-(1H-indol-3-yl)-1H-imidazol-4-yl)(3,4,5-trimethoxyphenyl)methanone (17ya); (2-(1H-indol-4-yl)-1H-imidazol-4-yl)(3,4,5-trimethoxyphenyl)methanone; (2-(1H-indol-5-yl)-1H-imidazol-4-yl)(3,4,5-trimethoxyphenyl)methanone; (2-(1H-indol-6-yl)-1H-imidazol-4-yl)(3,4,5-trimethoxyphenyl)methanone; or (2-(1H-indol-7-yl)-1H-imidazol-4-yl)(3,4,5-trimethoxyphenyl)methanone.

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula XXIa:

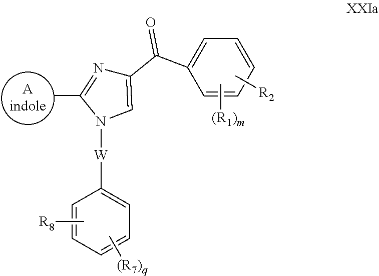

wherein
W is C═O, C═S, SO₂, or S═O;
A is indolyl optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₁ and R₂ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₇ and R₈ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
m is 1-4; and
q is 1-4; or a pharmaceutically acceptable salt, hydrate, polymorph, or isomer thereof.

Non limiting examples of CBSI compounds of Formula XXIa are selected from: (1-(phenylsulfonyl)-2-(1-(phenylsulfonyl)-2-(3,4,5-trimethoxybenzoyl)-1H-indol-5-yl)-1H-imidazol-4-yl)(3,4,5-trimethoxyphenyl)methanone (16xaa); or (1-(phenylsulfonyl)-2-(1-(phenylsulfonyl)-1H-indol-3-yl)-1H-imidazol-4-yl)(3,4,5-trimethoxyphenyl)methanone (17yaa).

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula XXII:

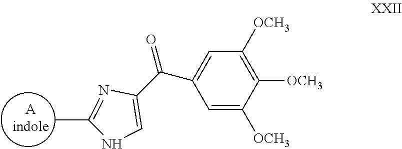

wherein
A is indolyl optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
or a pharmaceutically acceptable salt, hydrate, polymorph, or isomer thereof.

In one embodiment of the method of treating, reducing, inhibiting, slowing the progression of, promoting the regression of, or preventing atherosclerotic cardiovascular disease (ASCVD), A ring of compound of Formula XXII is substituted 5-indolyl. In another embodiment the substitution is —(C═O)-Aryl. In another embodiment, the aryl is 3,4,5-(OCH₃)₃-Ph. In another embodiment, A ring of compound of Formula XXII is 3-indolyl. Non limiting examples of CBSI compounds of Formula XXII are selected from: (5-(4-(3,4,5-trimethoxybenzoyl)-1H-imidazol-2-yl)-1H-indol-2-yl)(3,4,5-trimethoxyphenyl)methanone (15xaa); or (2-(1H-indol-3-yl)-1H-imidazol-4-yl)(3,4,5-trimethoxyphenyl)methanone (17ya).

The invention also encompasses methods of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of the Formula XXI or XXII represented by the structure of Formula 17ya:

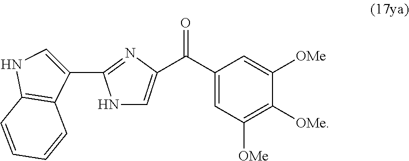 [SMILES: COc1cc(C(=O)c2c[nH]c(-c3c[nH]c4ccccc34)n2)cc(OC)c1OC]

In one embodiment of the method, R₄ and R₅ of compounds of formula XIII-XVI are hydrogens. Non-limiting examples of CBSI compounds of Formula XIII-XVI wherein R₄ and R₅ are hydrogens are selected from (2-phenyl-1H-imidazol-4-yl)(3,4,5-trimethoxyphenyl)methanone (12aa); (4-methoxyphenyl)(2-phenyl-1H-imidazol-4-yl)methanone (12ab); (3-methoxyphenyl)(2-phenyl-1H-imidazol-4-yl)methanone (12ac); (3,5-dimethoxyphenyl)(2-phenyl-1H-imidazol-4-yl)methanone (12ad); (3,4-dimethoxyphenyl)(2-phenyl-1H-imidazol-4-yl)methanone (12ae); (4-fluorophenyl)(2-phenyl-1H-imidazol-4-yl)methanone (12af); (3-fluorophenyl)(2-phenyl-1H-imidazol-4-yl)methanone (12ag); (2-phenyl-1H-imidazol-4-yl)(p-tolyl)methanone (12ah); or (2-phenyl-1H-imidazol-4-yl)(m-tolyl)methanone (12ai).

In many of the embodiments shown above, the method encompasses atherosclerotic cardiovascular disease (ASCVD) including any specific combination of the subtypes of ASCVD. In some embodiments, the invention encompasses at least one specific type of atherosclerotic cardiovascular disease (ASCVD) such as coronary artery disease (CAD) or peripheral artery disease (PAD). In one embodiment, the invention encompasses any specific combination of the subtypes of ASCVD. In some embodiments, the method of the invention encompasses at least coronary artery disease (CAD). In some embodiments, the method of the invention encompasses at least peripheral artery disease (PAD). In some embodiments, the method of the invention encompasses at least cerebrovascular accident (CVA) or transient ischemic accident (TIA). In some embodiments, the method of the invention encompasses abdominal aortic aneurysm (AAA). In some embodiments, the method of the invention encompasses atherosclerosis of the mesenteric arteries. In some embodiments, the method of the invention encompasses atherosclerosis of the renal arteries. In some embodiments, the method of the invention encompasses atherosclerosis of the carotid arteries. In some embodiments, the method of the invention encompasses atherosclerosis of the vertebral arteries (that supply blood to back of brain). In some embodiments, the invention only encompasses one specific type of atherosclerotic cardiovascular disease (ASCVD). In one embodiment, the invention only encompasses any one of CAD, PAD, CVA, TIA, AAA, or atherosclerosis of the mesenteric, renal, carotid, or vertebral arteries. In some embodiments, the invention encompasses at least one of the specific types of atherosclerotic cardiovascular disease (ASCVD) listed above. In some embodiments, the invention encompasses the method of treating, reducing, inhibiting, slowing the progression of, promoting the regression of, or preventing ASCVD, wherein the ASCVD is at least one of CAD or PAD. In some embodiments, the invention encompasses the method of treating, reducing, inhibiting, slowing the progression of, promoting the regression of, or preventing ASCVD, wherein the ASCVD is only CAD or only PAD.

In some embodiments, the methods of the invention involve determining the ‘progression’ or ‘regression’ of plaque [a single plaque, several different specified plaques, or collectively, all the plaque in the body]. In general, regression indicates that a disease's symptoms or signs partially or completely reverse from either the same patient before treatment began or as compared to a patient suffering from the same indication but has not received treatment with the compounds of the invention. E.g., a decreased tumor marker value is a sign that the tumor has regressed. Similarly in cardiovascular disease, lessened of signs and symptoms indicates regression of the underlying disease. Alternatively, monitoring plaque volume and/or character can be used to track the underlying disease. In general, progression indicates worsening or spreading in the body of a disease, suggesting that morbidity will increase over time. In ASCVD, the basic unit of the disease is the atherosclerotic plaque and monitoring progression or regression of ASCVD can be accomplished by monitoring the size and composition of said plaques. In some embodiments, the ‘progression’ or ‘regression’ of plaque will be determined by imaging said plaque using techniques such as computed tomography angiography (CTA) and particularly low attenuation plaque volume (LAPV) using CTA, as is discussed several places herein including Examples 1 and 2. In some embodiments, the terms “promotes the regression of” and “slows the progression of” plaque will be determined by imaging techniques used to visualize the plaque in a subject treated with a CBSI compound of the invention as compared to a subject not treated with a CBSI compound of the invention. In some embodiments, these imaging techniques used to determine whether the CBSI compound of the invention “promotes the regression of” or “slows the progression of” plaque will be computed tomography angiography (CTA) or coronary CTA (CCTA). In some embodiments, the CTA or CCTA imaging will allow low attenuation plaque volume (LAPV) to be monitored in said CBSI treated vs untreated groups, wherein LAPV correlates with plaque volume (size), as well as other parameters of the plaque as would be known by the skilled artisan. In the absence of the methods of the invention, atherosclerosis typically progresses as seen by increased absolute value of LAPV. In some embodiments, the method is expected to “promote the regression” of plaque, wherein treatment with the CBSI compound of the invention decreases the absolute value of LAPV (i.e., plaque volume observed to be smaller and/or plaque composition was more favorable) at the end of the CBSI treatment compared to baseline, whereas untreated group had no change (stable) LAPV or increased LAPV over the same period. In other embodiments, the method is expected to “slow the progression” of plaque, wherein treatment with the CBSI compound of the invention allows increases in the value of LAPV compared to baseline, but to a lesser extent compared to the increase in the untreated group over the same period of time (i.e., plaque volume and/or deleterious changes in plaque composition increased less due to treatment with CBSI compound of the invention than observed in a placebo treated group). Both “promotes regression” and “slows progression” represent beneficial outcomes of the administering of a CBSI compound of the invention. In some embodiments, the subjects will be receiving other drugs, cosmetics, foods, etc., in addition to a CBSI compound of the invention. In some embodiments, some of these other agents will be at least one of CYP inhibitors or inducers, CYP3A4 inhibitors or inducers, or PGP inhibitors or inducers. In some embodiments, these inhibitors, if dosed with colchicine, will be a liability for increasing colchicine blood levels, possibly to toxic levels, as will be discussed. In some embodiments, these inducers may be a liability for reducing colchicine (or typical ASCVD therapy) to subtherapeutic levels. In some embodiments, these other agents will have shared pharmacodynamic toxicity with colchicine, such as myotoxicity. In some embodiments, these patients will have renal or hepatic impairment, or other risk factors that exacerbate these drug-drug interaction liabilities. In some embodiments, the benefit of colchicine in terms of LAPV can be attained with a CBSI compound of the invention without the risk of drug-drug interactions associated with colchicine. In some embodiments, the benefit of colchicine in terms of LAPV can be attained with a CBSI compound of the invention without adverse events or death associated with colchicine.

In one embodiment, the invention encompasses the administration of a CBSI compound of the invention that slows the progression or promotes the regression of atheromatous plaques. In one embodiment, the invention encompasses the administration of a CBSI compound of the invention that slows the progression or promotes the regression of atheromatous plaques in a subject with ASCVD. In one embodiment, the CBSI compound of the invention slows the progression or promotes the regression of atheromatous plaques in a subject with CAD. In one embodiment, the CBSI compound of the invention slows the progression or promotes the regression of atheromatous plaques in a subject with PAD.

In one embodiment, the administration of a CBSI compound of the invention promotes the regression of atheromatous plaques as visualized by decreases of LAPV values. In some embodiments LAPV value changes for the treated group was about −5% to about −60%, or about −5% to about −50%, or about −5% to about −40%, or about −5% to about −30%, or about −10% to about −50%, or about −20% to about −50%, when compared to baseline of the same patients or when compared to untreated patients with the same indication.

In another embodiment, the administration of a CBSI compound of the invention promotes the regression of ASCVD or CAD or PAD as determined by decreases in proinflammatory cytokines or biomarkers of inflammation. In some embodiments, the proinflammatory cytokine is one of those known to be sensitive to a CBSI compound of the invention. In another embodiment, the administration of a CBSI compound of the invention promotes the regression of ASCVD or CAD or PAD as determined by decreases in the pro-inflammatory biomarker C-reactive protein (CRP) or high sensitivity CRP (hsCRP). Yet another embodiment of the invention encompasses methods of treating, reducing, inhibiting, slowing the progression of, promoting the regression of, or preventing atherosclerotic cardiovascular disease (ASCVD), or alternatively coronary artery disease (CAD), or alternatively peripheral artery disease (PAD), wherein said administering of said CBSI compound of this invention decreases blood levels of proinflammatory cytokines or other biomarkers of inflammation. In some of these embodiments, the proinflammatory cytokines and biomarkers are one or more of high sensitivity C-reactive protein (CRP), interferon (INF) γ, ferritin, interleukin (IL)-7, IL-15, IL-18, monocyte chemoattractant protein (MCP) 1, macrophage inflammatory protein (MIP)-1α, or MIP-1β. In some embodiments, the proinflammatory biomarker is CRP. In some embodiments, the high sensitivity CRP value changes for treated group was about −5% to about −60%, or about −5% to about −50%, or about −5% to about −40%, or about −5% to about −30%, or about −10% to about −50%, or about −20% to about −50%, when compared to baseline of the same patients or when compared to untreated patients with the same indication. In some cases, the administration of a CBSI compound of the invention promotes regression of one or more of atheromatous plaques (LAPV values, etc.), ASCVD related inflammation (CRP values, etc.), and signs/symptoms of disease (decreased angina, etc.).

In one embodiment, the administration of a CBSI compound of the invention slows the progression of atheromatous plaques as visualized by relative decreases of LAPV values compared to untreated patients of the same indication of about −5%, or about −10%, or about −15%, or about −20%, or about −25%, or about −30%, or about −35%, about −40%, or about −50%. In one embodiment, the administration of a CBSI compound of the invention slows the progression of ASCVD or CAD or PAD as determined by decreases in high sensitivity CRP (biomarker of inflammation) values. In some embodiments, the slowed progression is determined by relative decreases of high sensitivity CRP values compared to untreated patients of the same indication of about −5%, or about −10%, or about −15%, or about −20%, or about −25%, or about −30%, or about −35%, about −40%, or about −50%. In some cases, the administration of a CBSI compound of the invention slows progression of one or both atheromatous plaques (LAPV values) and ASCVD related inflammation (CRP values). In other embodiments, the administration of a CBSI compound of the invention promotes regression in terms of LAPV and slows progression in terms of CRP. In other embodiments, the administration of a CBSI compound of the invention promotes regression in terms symptomology of, eg., ACS, or some other ASCVD. Any one the above outcomes would be considered advantageous in terms of treatment of ASCVD and related diseases discussed herein.

In one embodiment, the invention encompasses the administration of a CBSI compound of the invention that treats, reduces, inhibits, slows the progression of, promotes the regression of, or prevents at least one of plaque initiation, plaque progression, plaque rupture, or plaque related thrombosis and its complications. In one embodiment, the invention encompasses the administration of a CBSI compound of the invention that treats, reduces, inhibits, slows the progression of, promotes the regression of, or prevents at least one of plaque initiation, plaque progression, plaque rupture, or plaque related thrombosis and its complications in a subject with ASCVD. In one embodiment, the invention encompasses the administration of a CBSI compound of the invention that treats, reduces, inhibits, slows the progression of, promotes the regression of, or prevents at least one of plaque initiation, plaque progression, plaque rupture, or plaque related thrombosis and its complications in a subject with CAD. In one embodiment, the invention encompasses the administration of a CBSI compound of the invention that treats, reduces, inhibits, slows the progression of, promotes the regression of, or prevents at least one of plaque initiation, plaque progression, plaque rupture, or plaque related thrombosis and its complications in a subject with PAD.

In some embodiments, the invention encompasses the administration of a CBSI compound of the invention to improve atheromatous plaque morphology or plaque composition. In one embodiment, the CBSI compound of the invention improves atheromatous plaque morphology or plaque composition in a subject with ASCVD. In one embodiment, the invention encompasses the administration of a CBSI compound of the invention to improve atheromatous plaque morphology or plaque composition in a subject with CAD. In one embodiment, the invention encompasses the administration of a CBSI compound of the invention to improve atheromatous plaque morphology or plaque composition in a subject with PAD.

In some embodiments, the invention encompasses the administration of a CBSI compound of the invention to improve plaque morphology or plaque composition by at least one of reducing plaque volume, decreasing plaque necrotic core size, increasing fibrous cap thickness, decreasing the extent of calcification of the plaque, or decreasing maladaptive plaque remodeling. In some embodiments, these improvements in plaque morphology or plaque composition occur in a subject with ASCVD. In some embodiments, these improvements occur in a subject with CAD. In some embodiments, these improvements occur in a subject with PAD.

In some embodiments, the methods of the invention encompass the administration of a CBSI compound of the invention to improve plaque morphology or plaque composition as measured by computed tomography angiography (CTA). In some embodiments, the methods of the invention encompass the administration of a CBSI compound of the invention to improve plaque morphology or plaque composition as measured by coronary computed tomography angiography (CCTA). In some embodiments, the improved plaque morphology or plaque composition is reduced plaque volume as compared to the non-treated subject having the same condition or the same subject before treatment. In some embodiments, the improved plaque morphology or plaque composition is observed by decreases of the low-attenuation plaque volume (LAPV). In some embodiments, LAPV is a key marker of unstable plaques prone to rupture and correlates with MACE. In some embodiments, the methods of the invention encompass the administration of a CBSI compound of the invention to decrease the blood levels of proinflammatory cytokines or proinflammatory biomarker. In some of these embodiments, the proinflammatory cytokine or biomarker one or more of high sensitivity C-reactive protein (CRP), interferon (INF) γ, ferritin, interleukin (IL)-7, IL-15, IL-18, monocyte chemoattractant protein (MCP) 1, macrophage inflammatory protein (MIP)-1α, or MIP-1β. In some of these embodiments, the proinflammatory biomarker is high sensitivity C-reactive protein (CRP). In one embodiment, the methods of the invention encompass the administration of a CBSI compound of the invention to decrease both low-attenuation plaque volume (LAPV) and blood levels of high sensitivity C-reactive protein (CRP).

In some embodiments, the methods of the invention encompass the administering of a CBSI compound of the invention that treats, inhibits, reduces the incidence of, or prevents adverse cardiovascular events that occur secondary to at least one of ASCVD, CAD, or PAD. In some embodiments, the methods of the invention encompass the administering of a CBSI compound of the invention that treats, inhibits, slows the progression of, promotes the regression of, or prevents acute coronary syndrome (ACS) that occurs secondary to at least one of ASCVD, CAD, or PAD. In some embodiments, the methods of the invention encompass the administering of a CBSI compound of the invention that treats, inhibits, reduces the incidence of, or prevents at least one of acute myocardial infarction (AMI), unstable angina (USA), chest pain or pressure, shortness of breath, excessive sweating, dizziness, or fatigue. In some embodiments, symptoms of ACS include acute myocardial infarction (AMI), unstable angina (USA), chest pain or pressure, shortness of breath, excessive sweating, dizziness, or fatigue.

In some embodiments, the methods of the invention encompass the administering of a CBSI compound of the invention that treats, inhibits, reduces the incidence of, or prevents major adverse cardiovascular events (MACE). In some embodiments, major adverse cardiovascular events include but are not limited to myocardial infarction, stroke, death, heart failure, hospitalization for heart failure, revascularization, e.g., percutaneous coronary intervention (PCI) or coronary artery bypass graft, arrhythmias, endocarditis, aortic dissection, or thrombotic events. In some embodiments, said MACE occurs secondary to at least one of ASCVD, CAD or PAD. In some embodiments, the methods of the invention treat, inhibit, reduce the incidence of, or prevent major adverse cardiovascular events (MACE) despite ongoing ACS or uncontrolled risk factors. In some embodiments, the uncontrolled risk factors include but are not limited to age, diabetes mellitus, hypertension, urologic conditions, chronic kidney disease, smoking, high LDL cholesterol, low HDL cholesterol, elevated triglycerides, family history of CAD, obesity, physical inactivity, or a history of previous cardiovascular events.

In some embodiments, the methods of the invention encompass the administering of a CBSI compound of the invention to a subject with at least one of ASCVD, CAD, or PAD, wherein said administering reverses physical function decline, or slows the progression of the decline in physical function. In some embodiments, said physical function is assessed by a six-minute walk test or stress test. In some embodiments, an outcome of reverses physical function decline indicates that the subject suffering from ASCVD, CAD, or PAD who is treated with a CBSI compound of the invention has improved physical function compared to said subject before treatment (at baseline). In some embodiments, slows the progression of physical function decline indicates that the subject suffering from ASCVD, CAD, or PAD who is treated with a CBSI compound of the invention has improved physical function compared to an untreated or placebo subject, but not better than said treated subject before treatment. In some embodiments, reverses physical function decline as assessed by six-minute walk would indicate that the subject suffering from ASCVD, CAD, or PAD who was treated with a CBSI compound of the invention is able to walk a further distance in six minutes, indicating better exercise tolerance, compared to said subject before treatment (at baseline). In some embodiments, slows the progression of physical function decline as assessed by six-minute walk would indicate that the subject suffering from ASCVD, CAD, or PAD who is treated with a CBSI compound of the invention is able to walk a further distance in six minutes, indicating better exercise tolerance, compared to an untreated or placebo treated subject, but not better than said treated subject before treatment (at baseline). In some embodiments, physical function is assessed by stress test as an indication of how well a person's heart handles exercise and workload, or to diagnose heart conditions. In some embodiments, physical function as assessed by stress test is measured in metabolic equivalents (MET), which are a less exact measurement of oxygen uptake per kilogram per minute. In some embodiments, physical function as assessed by stress test can reveal an outcome of reverses physical function decline in which the observed METs value is greater (indicating better exercise tolerance) following treatment with a CBSI of the invention than before said treatment. In some embodiments, physical function as assessed by stress test can reveal an outcome of slows progression of physical function decline in which the observed METs value is higher compared to an untreated or placebo treated subject, but not higher than said treated subject before treatment, indicating exercise tolerance that is intermediate between untreated subjects and treated subjects before treatment.

In some embodiments, the methods of the invention include the administering of a CBSI compound of the invention to provide the therapeutic benefit of colchicine to a subject with at least one of ASCVD, CAD, or PAD with reduced risk of, or without the risk of, the adverse events or death associated with colchicine. In some embodiments, the methods of the invention include the administering of a CBSI compound of the invention to provide the therapeutic benefit of colchicine to a subject with at least one of ASCVD, CAD, or PAD with reduced risk of, or without the risk of, the adverse events associated with colchicine; said adverse events include but are not limited to diarrhea, vomiting, abdominal cramps, neutropenia, blood dyscrasias, myalgia, rhabdomyolysis, or neurotoxicity.

In some embodiments, the methods of the invention include the administering of a CBSI compound of the invention to a subject in need thereof wherein said CBSI compound is administered in combination with other drugs for at least one of ASCVD, CAD, or PAD, the other drugs being necessitated by the risk factors and co-morbidities of the subject. In some embodiments, the cornerstone of medical therapy in ASCVD care is typically lipid-lowering agents combined with anti-platelet agents. In some embodiments, the CBSI compound of the invention is administered in combination with lipid-lowering and/or anti-platelet agents. In some embodiments, certain risk factors or co-morbidities are present indicating that further categories of pharmacotherapy need to be included in said ASCVD, CAD, or PAD care. In some embodiments, the other drugs include but are not limited to any or all of lipid-lowering agents and anti-platelet agents. In some embodiments, the other drugs include but are not limited to any or all of lipid-lowering agents and anti-platelet agents, and further additional anti-coagulants, anti-hypertensives, anti-anginal agents, and anti-diabetics. In some embodiments, said lipid-lowering agents include but are not limited to 3-hydroxy-3-methylglutaryl coenzyme A (HMG-CoA) reductase inhibitors (statins), proprotein convertase subtilisin/kexin type 9 (PCSK9) inhibitors, ezetimibe (Zetia), fish oil products, bempedoic acid, high dose icosapent ethyl, or vinacumab. In some embodiments, said combination of said CBSI compound of the invention and the other drugs can be administered with reduced risk of, or without the risk of, of drug-drug interactions between the CBSI compound of the invention and the other drugs. In some embodiments, said combination of said CBSI compound of the invention and said statin can be administered with reduced risk of, or without the risk of, of drug-drug interactions between the CBSI compound and the HMG-CoA reductase inhibitor (statin). In some embodiments, said combination of said CBSI compound of the invention and other drugs used for any of ASCVD, CAD, or PAD can be administered with reduced risk of, or without the risk of, of drug-drug interactions between the CBSI compound of the invention and said other drugs, wherein said other drugs include an HMG-CoA reductase inhibitor (statin). In some embodiments of the invention, the combination of CBSI of the invention and said other drugs used for any of ASCVD, CAD, or PAD is co-therapy, wherein the active pharmaceutical ingredients are not necessarily administered in a single formulation or at a single time. In some embodiments, other drugs may include drugs not indicated for ASCVD, CAD, or PAD but that may be inhibitors of PGP and/or CYP enzymes.

In some embodiments, the methods of the invention include the administering of a CBSI compound of the invention wherein said CBSI compound is administered in combination with other drugs used for at least one of ASCVD, CAD, or PAD with reduced risk of, or without the risk of, of said adverse events associated with colchicine, including but are not limited to diarrhea, vomiting, abdominal cramps, neutropenia, blood dyscrasias, myalgia, rhabdomyolysis, or neurotoxicity. In some embodiments, said CBSI compound of the invention or said co-therapy of the CBSI of the invention with other drugs in said subject, are given despite a history of intolerable adverse events of colchicine therapy in the subject.

In one embodiment, the method encompasses treating, reducing, inhibiting, slowing the progression of, promoting the regression of, or preventing coronary artery disease (CAD) with the CBSI compounds of the invention. In some embodiments, CAD is the atherosclerosis of the coronary arteries supplying blood to the heart. In some embodiments, CAD is also known as coronary heart disease and ischemic heart disease. In some embodiments, CAD is a chronic or stable ischemic heart disease referred to herein as “stable CAD”. In some embodiments, stable CAD is a chronic form wherein the coronary arteries are narrowed gradually over time. In some embodiments of stable CAD, the disease is asymptomatic or symptoms of decreased oxygen delivery to the heart are tolerable. In these embodiments, the coronary arteries gradually narrow over time and the heart receives less oxygen-rich blood. In some embodiments, CAD is an acute disease with sudden onset. In these embodiments, CAD is referred to as acute coronary syndrome (ACS). In these embodiments, ACS is a medical emergency caused by sudden rupture of coronary artery plaque(s) and/or formation of a blood clot that blocks blood flow to your heart. In some embodiments, ACS occurs with an abrupt blockage and causes a heart attack or acute myocardial infraction (AMI) and/or severe uncontrolled chest pain called unstable angina (USA).

The invention encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of Formula (I):

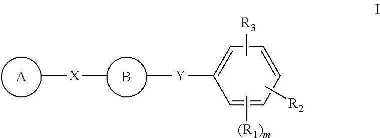

wherein
A is phenyl, indolyl, or indazolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole, thiazole, or benzimidazole, optionally substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond, NH, (C₁-C₄)alkyl, O, or S;
Y is a bond, —C═O, —C═S, SO₂, SO or S; and
m is 1-3, or a pharmaceutically acceptable salt, hydrate, polymorph, or isomer thereof.

Examples of CBSI compounds of Formula I include, but are not limited to, 2-phenyl-4-(3,4,5-trimethoxyphenyl)-1H-benzo[d]imidazole (3), 2-(1H-indol-3-yl)-4-(3,4,5-trimethoxyphenyl)-1H-benzo[d]imidazole (4), 2-(1H-indol-5-yl)-4-(3,4,5-trimethoxyphenyl)-1H-benzo[d]imidazole (5), or 3-(4-(3,4,5-trimethoxyphenyl)-1H-benzo[d]imidazol-2-yl)-1H-indazole (6).

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of Formula (II):

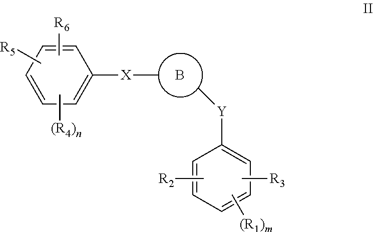

wherein
B is an imidazole, thiazole, or benzimidazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁, R₂, R₃, R₄, R₅ and R₆ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O or bond;
n is 1-3; and
m is 1-3; or a pharmaceutically acceptable salt, hydrate, polymorph, or isomer thereof.

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of Formula (III):

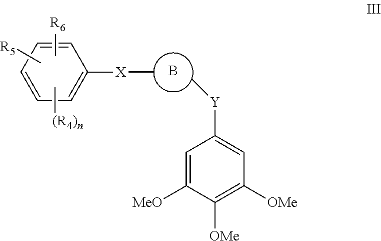

wherein
B is an imidazole, thiazole or benzimidazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₄, R and R₆ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O or bond; and
n is 1-3; or a pharmaceutically acceptable salt, hydrate, polymorph, or isomer thereof.

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of Formula (IV):

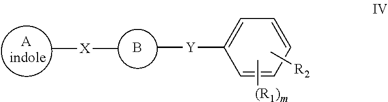

wherein
ring A is an indolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole or benzimidazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁ and R₂ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O or bond; and
m is 1-4; or a pharmaceutically acceptable salt, hydrate, polymorph, or isomer thereof.

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of Formula IV(a):

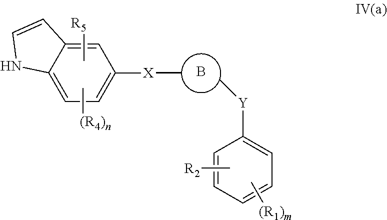

wherein
B is an imidazole or benzimidazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁, R₂, R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
X is a bond or NH;
Y is —C═O or bond;
n is 1-2; and
m is 1-4; or a pharmaceutically acceptable salt, hydrate, polymorph, or isomer thereof.

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of Formula (V):

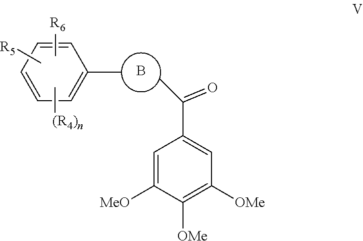

B is an imidazole or thiazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₄, R₅ and R₆ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
n is 1-3; or a pharmaceutically acceptable salt, hydrate, polymorph, or isomer thereof.

Examples of CBSI compounds of Formula V include, but are not limited to, (2-phenyl-thiazol-4-yl)-(3,4,5-trimethoxyphenyl)methanone (8f), [2-(3-fluorophenyl)-thiazol-4-yl](3,4,5-trimethoxyphenyl)methanone (8m), (2-(4-fluorophenyl)-thiazol-4-yl)-(3,4,5-trimethoxyphenyl)methanone (8n), (2-(3,4-dimethoxyphenyl)-thiazol-4-yl)-(3,4,5-trimethoxyphenyl)-methanone (8o), (2-(4-nitrophenyl)-thiazol-4-yl)-(3,4,5-trimethoxyphenyl)methanone (8p), 4-(4-(3,4,5-trimethoxybenzoyl)-thiazol-2-yl)-benzonitrile (8q), (2-(4-(trifluoromethyl)-phenyl)-thiazol-4-yl)(3,4,5-trimethoxyphenyl)methanone (8t), (2-(4-bromophenyl)-thiazol-4-yl)-(3,4,5-trimethoxyphenyl)methanone (8u), and (2-(4-ethylphenyl)-thiazol-4-yl)-(3,4,5-trimethoxy-phenyl)methanone (8v).

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula (VI):

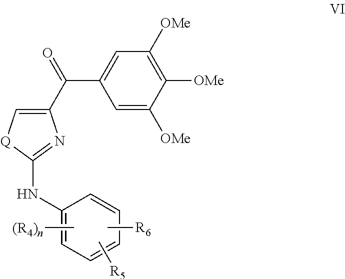

wherein
R₄, R₅ and R₆ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
Q is S or NH; and
n is 1-3; or a pharmaceutically acceptable salt, hydrate, polymorph, or isomer thereof.

Preferably, the variables for the compounds of Formula (VI) of R₄, R₅ and R₆ are independently hydrogen, (C₁-C₄)alkyl, halo(C₁-C₄)alkyl, O—(C₁-C₄)alkyl, O(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; Q is S or NH; and n is 1-3; or a pharmaceutically acceptable salt, hydrate, polymorph, or isomer thereof.

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine

TABLE 1

| Formula VI | Compound | R₄ | R₅ | R₆ | Q |
|---|---|---|---|---|---|
| 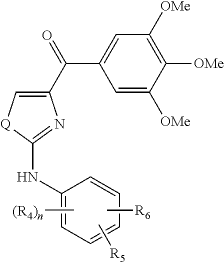 | 5e | H n = 1 | H | H | N |

Examples of CBSI compounds of Formula VI include, but are not limited to, (2-(phenylamino)thiazol-4-yl)(3,4,5-trimethoxyphenyl)methanone (5a), (2-(p-tolylamino)thiazol-4-yl)(3,4,5-trimethoxyphenyl)methanone (5b), (2-(p-fluorophenylamino)thiazol-4-yl)(3,4,5-trimethoxyphenyl)methanone (5c), or (2-((4-chlorophenyl)amino)thiazol-4-yl)(3,4,5-trimethoxyphenyl)methanone (5d). The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula VII:

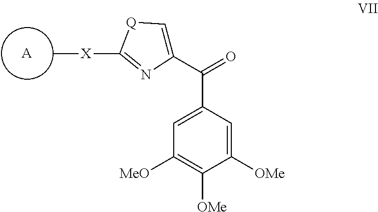

wherein
X is a bond or NH;
Q is S or NH; and
A is a phenyl, indolyl, or indazolyl ring, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer thereof.

Examples of CBSI compounds of Formula VII include, but are not limited to, (2-(phenylamino)-1H-imidazol-4-yl)(3,4,5-trimethoxyphenyl)methanone (5e), (2-(phenylamino)-1H-imidazol-4-yl)(3,4,5-trimethoxyphenyl)methanone hydrochloride salt (5He), or (2-(1H-indol-3-yl)-1H-imidazol-4-yl)(3,4,5-trimethoxyphenyl)methanone (17ya). Examples of CBSI compounds of Formula VII include, but are not limited to, (2-(phenylamino)thiazol-4-yl)(3,4,5-trimethoxyphenyl)methanone (5a), (2-(p-tolylamino)thiazol-4-yl)(3,4,5-trimethoxyphenyl)methanone (5b), (2-(p-fluorophenylamino)thiazol-4-yl)(3,4,5-trimethoxyphenyl)methanone (5c), or (2-((4-chlorophenyl)amino)thiazol-4-yl)(3,4,5-trimethoxyphenyl)methanone (5d).

Preferably, the variables in the compounds of Formula VII are X is a bond; Q is NH; and A is an indolyl ring optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer thereof.

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula VII(a):

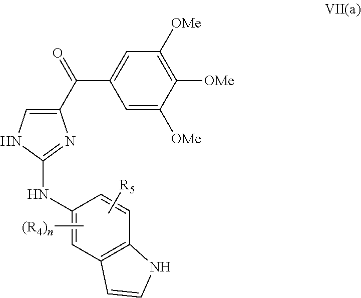

wherein
R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula VII(b):

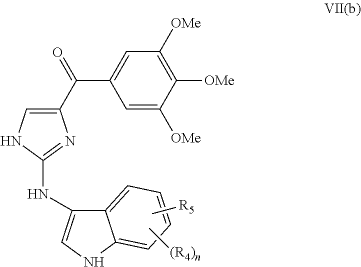

wherein
R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula VII(c):

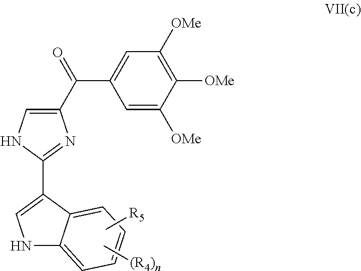

wherein
R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.
   Examples of CBSI compounds of Formula VII(c) include, but are not limited to, (2-(1H-indol-3-yl)-1H-imidazol-4-yl)(3,4,5-trimethoxyphenyl)methanone (17ya).

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula 17ya:

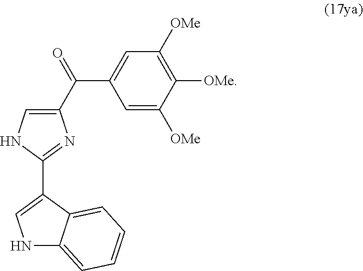 [SMILES: COc1cc(C(=O)c2c[nH]c(-c3c[nH]c4ccccc34)n2)cc(OC)c1OC]

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula XIII:

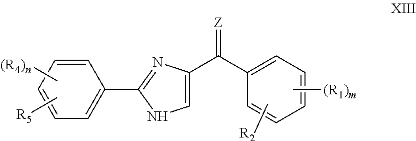

wherein
Z is O;
R₁ and R₄ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₂ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
m is an integer between 1-4; and
n is an integer between 1-4;
or a pharmaceutically acceptable salt, hydrate, polymorph, or isomer.

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula XIV:

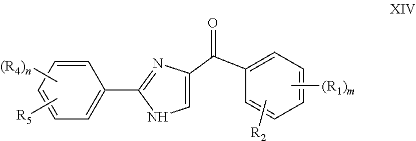

wherein
R₁ and R₄ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₂ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
m is an integer between 1-4; and
n is an integer between 1-4;
or a pharmaceutically acceptable salt, hydrate, polymorph, or isomer thereof.

Non limiting examples of CBSI compounds of Formula XIV are selected from: (2-phenyl-1H-imidazol-4-yl)(3,4,5-trimethoxyphenyl)methanone (12aa), (4-fluorophenyl)(2-phenyl-1H-imidazol-4-yl)methanone (12af), (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (4-fluorophenyl)(2-(4-methoxyphenyl)-1H-imidazol-4-yl)methanone (12cb), (2-(p-tolyl)-1H-imidazol-4-yl)(3,4,5-trimethoxyphenyl)methanone (12da), (4-fluorophenyl)(2-(p-tolyl)-1H-imidazol-4-yl)methanone (12db), (4-hydroxy-3,5-dimethoxyphenyl)(2-(p-tolyl)-1H-imidazol-4-yl)methanone (12dc), (2-(4-chlorophenyl)-1H-imidazol-4-yl)(3,4,5-trimethoxyphenyl)methanone (12fa), (2-(4-chlorophenyl)-1H-imidazol-4-yl)(4-fluorophenyl)methanone (12fb), (2-(4-chlorophenyl)-1H-imidazol-4-yl)(4-hydroxy-3,5-dimethoxyphenyl)methanone (12fc), (2-(4-(dimethylamino)phenyl)-1H-imidazol-4-yl)(3,4,5-trimethoxyphenyl)methanone (12ga), (2-(4-(dimethylamino)phenyl)-1H-imidazol-4-yl)(4-fluorophenyl)methanone (12gb), (2-(3,4-dimethoxyphenyl)-1H-imidazol-4-yl)(3,4,5-trimethoxyphenyl)methanone (12ha), (2-(4-(benzyloxy)phenyl)-1H-imidazol-4-yl)(4-fluorophenyl)methanone (12jb), (2-(4-bromophenyl)-1H-imidazol-4-yl)(3,4,5-trimethoxyphenyl)methanone (12la), or (2-(4-(trifluoromethyl)phenyl)-1H-imidazol-4-yl)(3,4,5-trimethoxyphenyl)methanone (12pa).

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula XIVa:

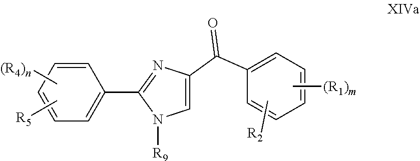

wherein
R₁ and R₄ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₂ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₉ is H, linear or branched alkyl, aryl, CH₂Ph, benzyl, haloalkyl, aminoalkyl, OCH₂Ph, SO₂-Aryl, —(C═O)-Aryl or OH, optionally substituted with at least one of hydrogen, hydroxyl, an aliphatic straight- or branched-chain C₁ to C₁₀ hydrocarbon, alkoxy, haloalkoxy, aryloxy, nitro, cyano, alkyl-CN, halo (e.g., F, Cl, Br, I), haloalkyl, dihaloalkyl, trihaloalkyl, COOH, C(O)Ph, C(O)-alkyl, C(O)O-alkyl, C(O)H, C(O)NH₂, —OC(O)CF₃, OCH₂Ph, amino, aminoalkyl, alkylamino, mesylamino, dialkylamino, arylamino, amido, NHC(O)-alkyl, urea, alkyl-urea, alkylamido (e.g., acetamide), haloalkylamido, arylamido, aryl, and C₅ to C₇ cycloalkyl, arylalkyl, and combinations thereof;
m is an integer between 1-4; and
n is an integer between 1-4;
or a pharmaceutically acceptable salt, hydrate, polymorph, or isomer thereof.

Non limiting examples of CBSI compounds of Formula XIVa are selected from: (4-fluorophenyl)(2-phenyl-1-(phenylsulfonyl)-1H-imidazol-4-yl)methanone (11af), (4-fluorophenyl)(2-(4-methoxyphenyl)-1-(phenylsulfonyl)-1H-imidazol-4-yl)methanone (11cb), (4-fluorophenyl)(1-(phenylsulfonyl)-2-(p-tolyl)-1H-imidazol-4-yl)methanone (11db), (2-(4-chlorophenyl)-1-(phenylsulfonyl)-1H-imidazol-4-yl)(4-fluorophenyl)methanone (11fb), (2-(4-(dimethylamino)phenyl)-1-(phenylsulfonyl)-1H-imidazol-4-yl)(3,4,5-trimethoxyphenyl)methanone (11ga), (2-(4-(dimethylamino)phenyl)-1-(phenylsulfonyl)-1H-imidazol-4-yl)(4-fluorophenyl)methanone (11gb), (2-(3,4-dimethoxyphenyl)-1-(phenylsulfonyl)-1H-imidazol-4-yl)(3,4,5-trimethoxyphenyl)methanone (11ha), (2-(4-(benzyloxy)phenyl)-1-(phenylsulfonyl)-1H-imidazol-4-yl)(4-fluorophenyl)methanone (11jb), (2-(4-(dimethylamino)phenyl)-1-((4-methoxyphenyl)sulfonyl)-1H-imidazol-4-yl)(4-fluorophenyl)methanone (12gba), (1-benzyl-2-(p-tolyl)-1H-imidazol-4-yl)(3,4,5-trimethoxyphenyl)methanone (12daa), (1-methyl-2-(p-tolyl)-1H-imidazol-4-yl)(3,4,5-trimethoxyphenyl)methanone (12dab), or (4-fluorophenyl)(2-(4-methoxyphenyl)-1-methyl-1H-imidazol-4-yl)methanone (12cba).

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula XV:

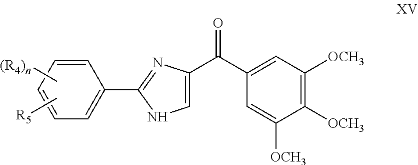

wherein
R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.

Non limiting examples of CBSI compounds of Formula XV are selected from: (2-phenyl-1H-imidazol-4-yl)(3,4,5-trimethoxyphenyl)methanone (12aa), (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (2-(p-tolyl)-1H-imidazol-4-yl)(3,4,5-trimethoxyphenyl)methanone (12da), (3,4,5-trimethoxyphenyl)(2-(3,4,5-trimethoxyphenyl)-1H-imidazol-4-yl)methanone (12ea), (2-(4-chlorophenyl)-1H-imidazol-4-yl)(3,4,5-trimethoxyphenyl)methanone (12fa), (2-(4-(dimethylamino)phenyl)-1H-imidazol-4-yl)(3,4,5-trimethoxyphenyl)methanone (12ga), (2-(3,4-dimethoxyphenyl)-1H-imidazol-4-yl)(3,4,5-trimethoxyphenyl)methanone (12ha), (2-(2-(trifluoromethyl)phenyl)-1H-imidazol-4-yl)(3,4,5-trimethoxyphenyl)methanone (12ia), (2-(4-(benzyloxy)phenyl)-1H-imidazol-4-yl)(3,4,5-trimethoxyphenyl)methanone (12ja), (2-(4-hydroxyphenyl)-1H-imidazol-4-yl)(3,4,5-trimethoxyphenyl)methanone (12ka), (2-(4-bromophenyl)-1H-imidazol-4-yl)(3,4,5-trimethoxyphenyl)methanone (12la), or (2-(4-(trifluoromethyl)phenyl)-1H-imidazol-4-yl)(3,4,5-trimethoxyphenyl)methanone (12pa).

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula XVI:

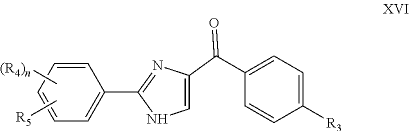

wherein
R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₃ is I, Br, Cl, or F; and
n is 1-4; or a pharmaceutically acceptable salt, hydrate, polymorph, or isomer.

Non limiting examples of CBSI compounds of Formula XVI are selected from: (4-fluorophenyl)(2-phenyl-1H-imidazol-4-yl)methanone (12af), (4-fluorophenyl)(2-(4-methoxyphenyl)-1H-imidazol-4-yl)methanone (12cb), (4-fluorophenyl)(2-(p-tolyl)-1H-imidazol-4-yl)methanone (12db), 4-fluorophenyl)(2-(3,4,5-trimethoxyphenyl)-1H-imidazol-4-yl)methanone (12eb), (2-(4-chlorophenyl)-1H-imidazol-4-yl)(4-fluorophenyl)methanone (12fb), (2-(4-(dimethylamino)phenyl)-1H-imidazol-4-yl)(4-fluorophenyl)methanone (12gb), or (2-(4-(benzyloxy)phenyl)-1H-imidazol-4-yl)(4-fluorophenyl)methanone (12jb).

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula XVII:

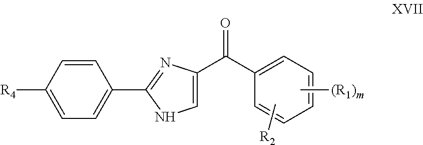

wherein
R₄ is H, O—(C₁-C₄)alkyl, I, Br, Cl, F, (C₁-C₄)alkyl, halo(C₁-C₄)alkyl, amino(C₁-C₄)alkyl, OCH₂Ph, OH, CN, NO₂, —NHCO—(C₁-C₄)alkyl, COOH, C(O)O—(C₁-C₄)alkyl or C(O)H; wherein R₁ and R₂ are independently H, O—(C₁-C₄)alkyl, I, Br, Cl, F, (C₁-C₄)alkyl, halo(C₁-C₄)alkyl, amino(C₁-C₄)alkyl, OCH₂Ph, OH, CN, NO₂, —NHCO—(C₁-C₄)alkyl, COOH, C(O)O—(C₁-C₄)alkyl or C(O)H; and
m is 1-4; or a pharmaceutically acceptable salt, hydrate, polymorph, or isomer thereof.

Non limiting examples of CBSI compounds of Formula XVII are selected from: (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (4-fluorophenyl)(2-(4-methoxyphenyl)-1H-imidazol-4-yl)methanone (12cb), (2-(p-tolyl)-1H-imidazol-4-yl)(3,4,5-trimethoxyphenyl)methanone (12da), (4-fluorophenyl)(2-(p-tolyl)-1H-imidazol-4-yl)methanone (12db), (4-hydroxy-3,5-dimethoxyphenyl)(2-(p-tolyl)-1H-imidazol-4-yl)methanone (12dc), (2-(4-chlorophenyl)-1H-imidazol-4-yl)(3,4,5-trimethoxyphenyl)methanone (12fa), (2-(4-chlorophenyl)-1H-imidazol-4-yl)(4-fluorophenyl)methanone (12fb), (2-(4-chlorophenyl)-1H-imidazol-4-yl)(3,4,5-trihydroxyphenyl)methanone (13fa), (2-(4-(dimethylamino)phenyl)-1H-imidazol-4-yl)(3,4,5-trimethoxyphenyl)methanone (12ga), (2-(4-(dimethylamino)phenyl)-1H-imidazol-4-yl)(4-fluorophenyl)methanone (12gb), (2-(4-(benzyloxy)phenyl)-1H-imidazol-4-yl)(4-fluorophenyl)methanone (12jb), (2-(4-hydroxyphenyl)-1H-imidazol-4-yl)(3,4,5-trimethoxyphenyl)methanone (12ka), (2-(4-bromophenyl)-1H-imidazol-4-yl)(3,4,5-trimethoxyphenyl)methanone (12la), or (2-(4-(trifluoromethyl)phenyl)-1H-imidazol-4-yl)(3,4,5-trimethoxyphenyl)methanone (12pa).

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site

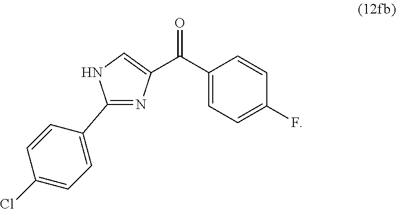 [SMILES: O=C(c1ccc(F)cc1)c1c[nH]c(-c2ccc(Cl)cc2)n1]

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula XVII represented by the structure of formula 12cb:

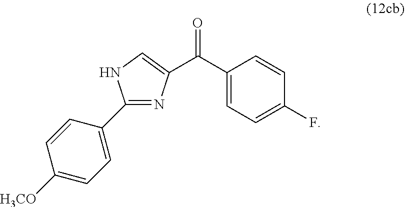 [SMILES: COc1ccc(-c2nc(C(=O)c3ccc(F)cc3)c[nH]2)cc1]

Non limiting examples of CBSI compounds of this invention are selected from: (4-methoxyphenyl)(2-phenyl-1H-imidazol-1-yl)methanone (12aba), (2-phenyl-1H-imidazol-1-yl)(3,4,5-trimethoxyphenyl)methanone (12aaa), 2-phenyl-1-(phenylsulfonyl)-1H-imidazole (10a), 2-(4-nitrophenyl)-1-(phenylsulfonyl)-1H-imidazole (10x), 2-(4-(benzyloxy)phenyl)-1-(phenylsulfonyl)-1H-imidazole (10j), or 2-(2H-indol-5-yl)-4-(3,4,5-trimethoxyphenyl)-1H-imidazo[4,5-c]pyridine (7).

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula XIX:

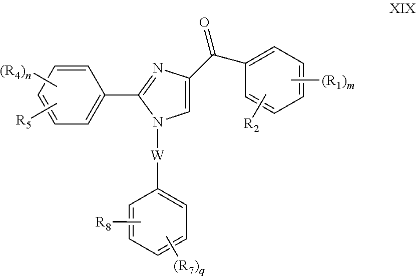

wherein
W is C═O, C═S, SO₂, or S═O;
R₁, R₄ and R₇ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₂, R₅ and R₈ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
m is 1-4;
n is 1-4; and
q is 1-4;
or its pharmaceutically acceptable salt, hydrate, polymorph, or isomer.

Non limiting examples of CBSI compounds of Formula XIX are selected from: (2-(4-(dimethylamino)phenyl)-1-((4-methoxyphenyl)sulfonyl)-1H-imidazol-4-yl)(3,4,5-trimethoxyphenyl)methanone (11gaa); (2-(4-bromophenyl)-1-(phenylsulfonyl)-1H-imidazol-4-yl)(3,4,5-trimethoxyphenyl)methanone (11la), (4-fluorophenyl)(2-(4-methoxyphenyl)-1-(phenylsulfonyl)-1H-imidazol-4-yl)methanone (11cb), (2-(4-chlorophenyl)-1-(phenylsulfonyl)-1H-imidazol-4-yl)(4-fluorophenyl)methanone (11fb), (4-fluorophenyl)(2-phenyl-1-(phenylsulfonyl)-1H-imidazol-4-yl)methanone (11af), (4-fluorophenyl)(1-(phenylsulfonyl)-2-(p-tolyl)-1H-imidazol-4-yl)methanone (11db), (2-(4-(dimethylamino)phenyl)-1-(phenylsulfonyl)-1H-imidazol-4-yl)(3,4,5-trimethoxyphenyl)methanone (11ga), (2-(4-(dimethylamino)phenyl)-1-(phenylsulfonyl)-1H-imidazol-4-yl)(4-fluorophenyl)methanone (11gb), (2-(3,4-dimethoxyphenyl)-1-(phenylsulfonyl)-1H-imidazol-4-yl)(3,4,5-trimethoxyphenyl)methanone (11ha), (2-(4-(benzyloxy)phenyl)-1-(phenylsulfonyl)-1H-imidazol-4-yl)(4-fluorophenyl)methanone (11jb), or (2-(4-(dimethylamino)phenyl)-1-((4-methoxyphenyl)sulfonyl)-1H-imidazol-4-yl)(4-fluorophenyl)methanone (12gba).

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula XIX represented by the structure of formula 11cb:

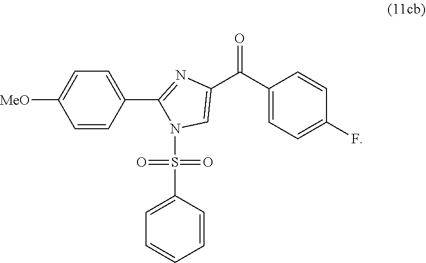 [SMILES: COc1ccc(-c2nc(C(=O)c3ccc(F)cc3)cn2S(=O)(=O)c2ccccc2)cc1]

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula XIX represented by the structure of formula 11fb:

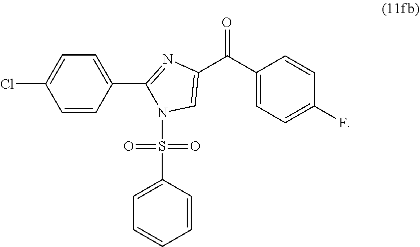 [SMILES: O=C(c1ccc(F)cc1)c1cn(S(=O)(=O)c2ccccc2)c(-c2ccc(Cl)cc2)n1]

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula XX:

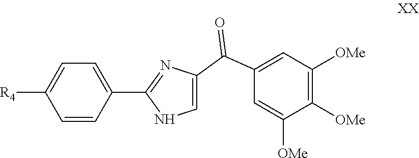

wherein
R₄ is independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer.

Non limiting examples of CBSI compounds of Formula XX are selected from: (2-phenyl-1H-imidazol-4-yl)(3,4,5-trimethoxyphenyl)methanone (12aa), (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (2-(p-tolyl)-1H-imidazol-4-yl)(3,4,5-trimethoxyphenyl)methanone (12da), (2-(4-chlorophenyl)-1H-imidazol-4-yl)(3,4,5-trimethoxyphenyl)methanone (12fa), (2-(4-(dimethylamino)phenyl)-1H-imidazol-4-yl)(3,4,5-trimethoxyphenyl)methanone (12ga), (2-(2-(trifluoromethyl)phenyl)-1H-imidazol-4-yl)(3,4,5-trimethoxyphenyl)methanone (12ia), (2-(4-(benzyloxy)phenyl)-1H-imidazol-4-yl)(3,4,5-trimethoxyphenyl)methanone (12ja), (2-(4-hydroxyphenyl)-1H-imidazol-4-yl)(3,4,5-trimethoxyphenyl)methanone (12ka), (2-(4-bromophenyl)-1H-imidazol-4-yl)(3,4,5-trimethoxyphenyl)methanone (12la), or (2-(4-(trifluoromethyl)phenyl)-1H-imidazol-4-yl)(3,4,5-trimethoxyphenyl)methanone (12pa).

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula XX represented by the structure of formula 12da:

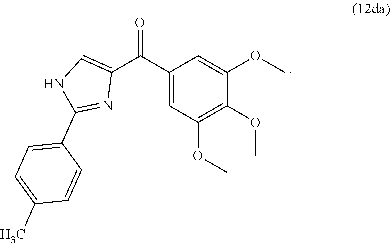 [SMILES: COc1cc(C(=O)c2c[nH]c(-c3ccc(C)cc3)n2)cc(OC)c1OC ; "."]

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula XX represented by the structure of formula 12fa:

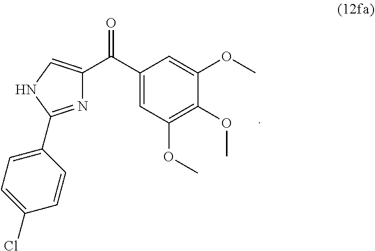 [SMILES: COc1cc(C(=O)c2c[nH]c(-c3ccc(Cl)cc3)n2)cc(OC)c1OC]

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula XXI:

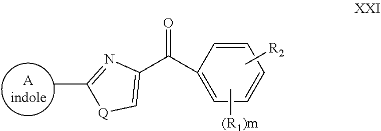

wherein
A is indolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
Q is S or NH;
R₁ and R₂ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
m is 1-4; or a pharmaceutically acceptable salt, hydrate, polymorph, or isomer thereof.

In one embodiment of the method of treating, reducing, inhibiting, slowing the progression of, promoting the regression of, or preventing coronary artery disease (CAD), A ring of compound of Formula XXI is substituted 5-indolyl. In another embodiment the substitution is —(C═O)-Aryl. In another embodiment, the aryl is 3,4,5-(OCH₃)₃-Ph. In another embodiment, A ring of compound of Formula XXI is 3-indolyl. In another embodiment, A ring of compound of Formula XXI is 5-indolyl. In another embodiment, A ring of compound of Formula XXI is 2-indolyl. Non limiting examples of CBSI compounds of Formula XXI are selected from: (5-(4-(3,4,5-trimethoxybenzoyl)-1H-imidazol-2-yl)-1H-indol-2-yl)(3,4,5-trimethoxyphenyl)methanone (15xaa); (1-(phenylsulfonyl)-2-(1-(phenylsulfonyl)-2-(3,4,5-trimethoxybenzoyl)-1H-indol-5-yl)-1H-imidazol-4-yl)(3,4,5-trimethoxyphenyl)methanone (16xaa); (2-(1H-indol-3-yl)-1H-imidazol-4-yl)(3,4,5-trimethoxyphenyl)methanone (17ya); (2-(1H-indol-2-yl)thiazol-4-yl)(3,4,5-trimethoxyphenyl)methanone (62a); or (2-(1H-indol-5-yl)thiazol-4-yl)(3,4,5-trimethoxyphenyl)methanone (66a).

A particularly preferred method of treating, reducing, inhibiting, slowing the progression of, promoting the regression of, or preventing coronary artery disease (CAD) of the invention uses at least one CBSI compound of Formula XXI including (2-(1H-indol-1-yl)-1H-imidazol-4-yl)(3,4,5-trimethoxyphenyl)methanone; (2-(1H-indol-2-yl)-1H-imidazol-4-yl)(3,4,5-trimethoxyphenyl)methanone; (2-(1H-indol-3-yl)-1H-imidazol-4-yl)(3,4,5-trimethoxyphenyl)methanone (17ya); (2-(1H-indol-4-yl)-1H-imidazol-4-yl)(3,4,5-trimethoxyphenyl)methanone; (2-(1H-indol-5-yl)-1H-imidazol-4-yl)(3,4,5-trimethoxyphenyl)methanone; (2-(1H-indol-6-yl)-1H-imidazol-4-yl)(3,4,5-trimethoxyphenyl)methanone; or (2-(1H-indol-7-yl)-1H-imidazol-4-yl)(3,4,5-trimethoxyphenyl)methanone.

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula XXIa:

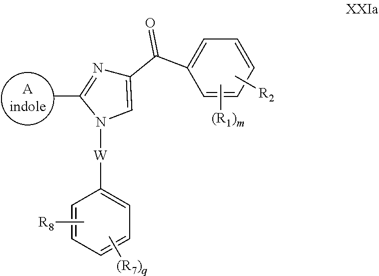

wherein
W is C═O, C═S, SO₂, or S═O;
A is indolyl optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₁ and R₂ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₇ and R₈ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
m is 1-4; and q is 1-4; or a pharmaceutically acceptable salt, hydrate, polymorph, or isomer thereof.

Non limiting examples of CBSI compounds of Formula XXIa are selected from: (1-(phenylsulfonyl)-2-(1-(phenylsulfonyl)-2-(3,4,5-trimethoxybenzoyl)-1H-indol-5-yl)-1H-imidazol-4-yl)(3,4,5-trimethoxyphenyl)methanone (16xaa); or (1-(phenylsulfonyl)-2-(1-(phenylsulfonyl)-1H-indol-3-yl)-1H-imidazol-4-yl)(3,4,5-trimethoxyphenyl)methanone (17yaa).

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula XXII:

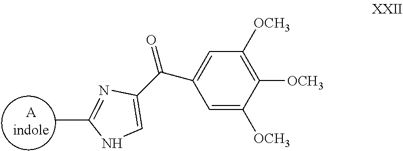

wherein
A is indolyl optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
or a pharmaceutically acceptable salt, hydrate, polymorph, or isomer thereof.

In one embodiment of the method of treating, reducing, inhibiting, slowing the progression of, promoting the regression of, or preventing coronary artery disease (CAD), A ring of compound of Formula XXII is substituted 5-indolyl. In another embodiment the substitution is —(C═O)-Aryl. In another embodiment, the aryl is 3,4,5-(OCH₃)₃-Ph. In another embodiment, A ring of compound of Formula XXII is 3-indolyl. Non limiting examples of CBSI compounds of Formula XXII are selected from: (5-(4-(3,4,5-trimethoxybenzoyl)-1H-imidazol-2-yl)-1H-indol-2-yl)(3,4,5-trimethoxyphenyl)methanone (15xaa); or (2-(1H-indol-3-yl)-1H-imidazol-4-yl)(3,4,5-trimethoxyphenyl)methanone (17ya).

The invention also encompasses methods of treating, reducing, inhibiting, slowing the progression of, promoting the regression of, or preventing coronary artery disease (CAD) in a subject in need thereof by administering to the subject a formulation having a therapeutically effective amount of a colchicine binding site inhibitor (CBSI) compound of the Formula XXI or XXII represented by the structure of Formula 17ya:

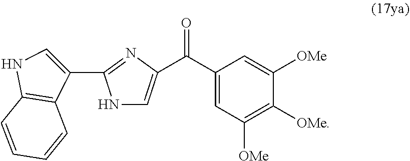 [SMILES: COc1cc(C(=O)c2c[nH]c(-c3c[nH]c4ccccc34)n2)cc(OC)c1OC]

In one embodiment of the method, R₄ and R₅ of compounds of formula XIII-XVI are hydrogens. Non-limiting examples of CBSI compounds of Formula XIII-XVI wherein R₄ and R₅ are hydrogens are selected from (2-phenyl-1H-imidazol-4-yl)(3,4,5-trimethoxyphenyl)methanone (12aa); (4-methoxyphenyl)(2-phenyl-1H-imidazol-4-yl)methanone (12ab); (3-methoxyphenyl)(2-phenyl-1H-imidazol-4-yl)methanone (12ac); (3,5-dimethoxyphenyl)(2-phenyl-1H-imidazol-4-yl)methanone (12ad); (3,4-dimethoxyphenyl)(2-phenyl-1H-imidazol-4-yl)methanone (12ae); (4-fluorophenyl)(2-phenyl-1H-imidazol-4-yl)methanone (12af); (3-fluorophenyl)(2-phenyl-1H-imidazol-4-yl)methanone (12ag); (2-phenyl-1H-imidazol-4-yl)(p-tolyl)methanone (12ah); or (2-phenyl-1H-imidazol-4-yl)(m-tolyl)methanone (12ai).

In one embodiment, the method encompasses treating, reducing, inhibiting, slowing the progression of, promoting the regression of, or preventing peripheral artery disease (PAD) with a CBSI compound of the invention. In some embodiments, PAD is the narrowing of the inside of the peripheral arteries that carry blood away from the heart to other parts of the body. In some embodiments, PAD is caused by atherosclerosis of the peripheral arteries supplying blood to the limbs and lower extremities. In some embodiments, PAD is asymptomatic. In other embodiments, PAD has symptoms that include intermittent claudication which is muscle pain in a limb (usually a leg) that resolves with rest. In other embodiments, PAD progresses to critical limb ischemia (CLI) which is severe and life-threatening form of PAD characterized by ischemic rest pain, a burning pain or numbness in the legs or feet, ulcers or sores on the legs or feet, gangrene, or discoloration, coldness, numbness, or tingling in the lower legs and feet. In some embodiments, PAD progresses to atherosclerosis of the heart (CAD), brain (CVA or TIA), or limbs (CLI).

The invention encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of Formula (I):

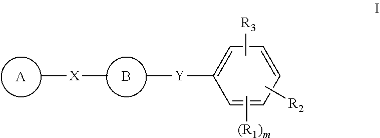

wherein
A is phenyl, indolyl, or indazolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole, thiazole, or benzimidazole, optionally substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond, NH, (C₁-C₄)alkyl, O, or S;
Y is a bond, —C═O, —C═S, SO₂, SO or S; and
m is 1-3, or a pharmaceutically acceptable salt, hydrate, polymorph, or isomer thereof.

Examples of CBSI compounds of Formula I include, but are not limited to, 2-phenyl-4-(3,4,5-trimethoxyphenyl)-1H-benzo[d]imidazole (3), 2-(1H-indol-3-yl)-4-(3,4,5-trimethoxyphenyl)-1H-benzo[d]imidazole (4), 2-(1H-indol-5-yl)-4-(3,4,5-trimethoxyphenyl)-1H-benzo[d]imidazole (5), or 3-(4-(3,4,5-trimethoxyphenyl)-1H-benzo[d]imidazol-2-yl)-1H-indazole (6).

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of Formula (II):

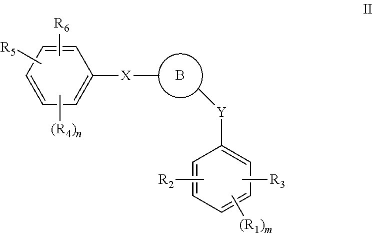

wherein
B is an imidazole, thiazole, or benzimidazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁, R₂, R₃, R₄, R₅ and R₆ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O or bond;
n is 1-3; and
m is 1-3; or a pharmaceutically acceptable salt, hydrate, polymorph, or isomer thereof.

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of Formula (III):

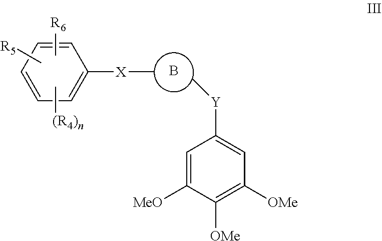

wherein
B is an imidazole, thiazole or benzimidazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₄, R₅ and R₆ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O or bond; and
n is 1-3; or a pharmaceutically acceptable salt, hydrate, polymorph, or isomer thereof.

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of Formula (IV):

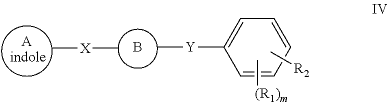

ring A is an indolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole or benzimidazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁ and R₂ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O or bond; and
m is 1-4; or a pharmaceutically acceptable salt, hydrate, polymorph, or isomer thereof.

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of Formula IV(a):

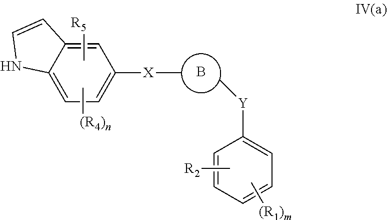

wherein
B is an imidazole or benzimidazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁, R₂, R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
X is a bond or NH;
Y is —C═O or bond;
n is 1-2; and
m is 1-4; or a pharmaceutically acceptable salt, hydrate, polymorph, or isomer thereof.

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of Formula (V):

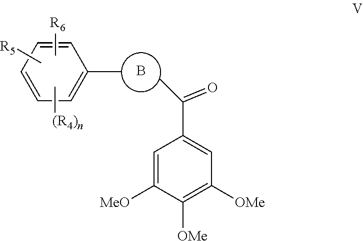

wherein
B is an imidazole or thiazole, optionally independently substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₄, R₅ and R₆ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
n is 1-3; or a pharmaceutically acceptable salt, hydrate, polymorph, or isomer thereof. Examples of CBSI compounds of Formula V include, but are not limited to, (2-phenyl-thiazol-4-yl)-(3,4,5-trimethoxyphenyl)methanone (8f), [2-(3-fluorophenyl)-thiazol-4-yl](3,4,5-trimethoxyphenyl)methanone (8m), (2-(4-fluorophenyl)-thiazol-4-yl)-(3,4,5-trimethoxyphenyl)methanone (8n), (2-(3,4-dimethoxyphenyl)-thiazol-4-yl)-(3,4,5-trimethoxyphenyl)-methanone (8o), (2-(4-nitrophenyl)-thiazol-4-yl)-(3,4,5-trimethoxyphenyl)methanone (8p), 4-(4-(3,4,5-trimethoxybenzoyl)-thiazol-2-yl)-benzonitrile (8q), (2-(4-(trifluoromethyl)-phenyl)-thiazol-4-yl)(3,4,5-trimethoxyphenyl)methanone (8t), (2-(4-bromophenyl)-thiazol-4-yl)-(3,4,5-trimethoxyphenyl)methanone (8u), and (2-(4-ethylphenyl)-thiazol-4-yl)-(3,4,5-trimethoxy-phenyl)methanone (8v).

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula (VI):

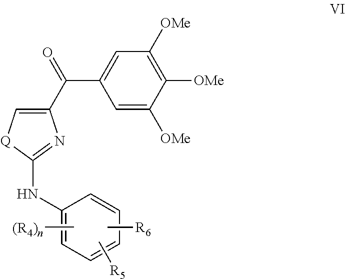

wherein
R₄, R₅ and R₆ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
Q is S or NH; and
n is 1-3; or a pharmaceutically acceptable salt, hydrate, polymorph, or isomer thereof.

Preferably, the variables for the compounds of Formula (VI) for R₄, R₅ and R₆ are independently hydrogen, (C₁-C₄)alkyl, halo(C₁-C₄)alkyl, O—(C₁-C₄)alkyl, O(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; Q is S or NH; and n is 1-3; or a pharmaceutically acceptable salt, hydrate, polymorph, or isomer thereof.

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine

TABLE 1

| Formula VI | Compound | R₄ | R₅ | R₆ | Q |
|---|---|---|---|---|---|
| 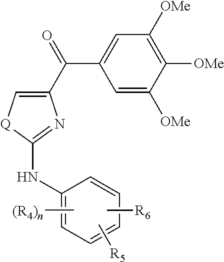 | 5e | H n = 1 | H | H | N |

Examples of CBSI compounds of Formula VI include, but are not limited to, (2-(phenylamino)thiazol-4-yl)(3,4,5-trimethoxyphenyl)methanone (5a), (2-(p-tolylamino)thiazol-4-yl)(3,4,5-trimethoxyphenyl)methanone (5b), (2-(p-fluorophenylamino)thiazol-4-yl)(3,4,5-trimethoxyphenyl)methanone (5c), or (2-((4-chlorophenyl)amino)thiazol-4-yl)(3,4,5-trimethoxyphenyl)methanone (5d).

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula VII:

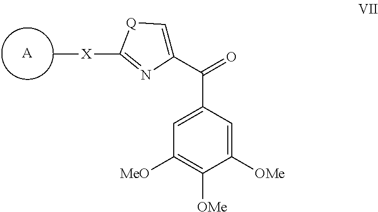

wherein
X is a bond or NH;
Q is S or NH; and
A is a phenyl, indolyl, or indazolyl ring, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer thereof.

Examples of CBSI compounds of Formula VII include, but are not limited to, (2-(phenylamino)-1H-imidazol-4-yl)(3,4,5-trimethoxyphenyl)methanone (5e), (2-(phenylamino)-1H-imidazol-4-yl)(3,4,5-trimethoxyphenyl)methanone hydrochloride salt (5He), (2-(1H-indol-3-yl)-1H-imidazol-4-yl)(3,4,5-trimethoxyphenyl)methanone (17ya), (2-(phenylamino)thiazol-4-yl)(3,4,5-trimethoxyphenyl)methanone (5a), (2-(p-tolylamino)thiazol-4-yl)(3,4,5-trimethoxyphenyl)methanone (5b), (2-(p-fluorophenylamino)thiazol-4-yl)(3,4,5-trimethoxyphenyl)methanone (5c), or (2-((4-chlorophenyl)amino)thiazol-4-yl)(3,4,5-trimethoxyphenyl)methanone (5d).

Preferably, the variables in the compounds of Formula VII are X is a bond; Q is NH; and A is an indolyl ring optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer thereof.

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula VII(a):

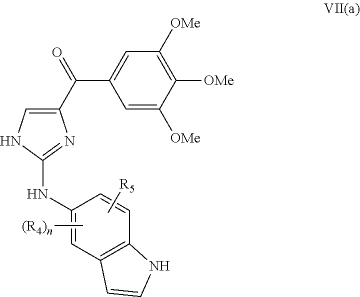

wherein
R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula VII(b):

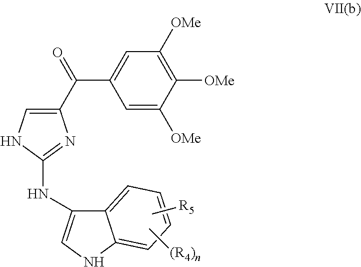

wherein
R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula VII(c):

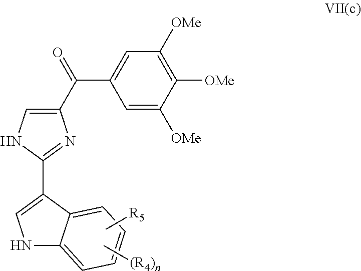

wherein
R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.
   Examples of CBSI compounds of Formula VII(c) include, but are not limited to, (2-(1H-indol-3-yl)-1H-imidazol-4-yl)(3,4,5-trimethoxyphenyl)methanone (17ya).

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula 17ya:

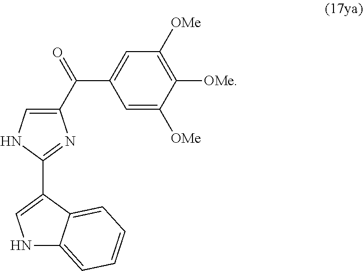 [SMILES: COc1cc(C(=O)c2c[nH]c(-c3c[nH]c4ccccc34)n2)cc(OC)c1OC]

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula XIII:

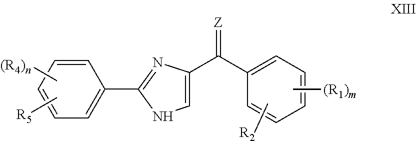

wherein
Z is O;
R₁ and R₄ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₂ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
m is an integer between 1-4; and
n is an integer between 1-4;
or a pharmaceutically acceptable salt, hydrate, polymorph, or isomer.

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula XIV:

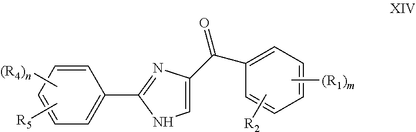

wherein
R₁ and R₄ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₂ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
m is an integer between 1-4; and
n is an integer between 1-4;
or a pharmaceutically acceptable salt, hydrate, polymorph, or isomer thereof.

Non limiting examples of CBSI compounds of Formula XIV are selected from: (2-phenyl-1H-imidazol-4-yl)(3,4,5-trimethoxyphenyl)methanone (12aa), (4-fluorophenyl)(2-phenyl-1H-imidazol-4-yl)methanone (12af), (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (4-fluorophenyl)(2-(4-methoxyphenyl)-1H-imidazol-4-yl)methanone (12cb), (2-(p-tolyl)-1H-imidazol-4-yl)(3,4,5-trimethoxyphenyl)methanone (12da), (4-fluorophenyl)(2-(p-tolyl)-1H-imidazol-4-yl)methanone (12db), (4-hydroxy-3,5-dimethoxyphenyl)(2-(p-tolyl)-1H-imidazol-4-yl)methanone (12dc), (2-(4-chlorophenyl)-1H-imidazol-4-yl)(3,4,5-trimethoxyphenyl)methanone (12fa), (2-(4-chlorophenyl)-1H-imidazol-4-yl)(4-fluorophenyl)methanone (12fb), (2-(4-chlorophenyl)-1H-imidazol-4-yl)(4-hydroxy-3,5-dimethoxyphenyl)methanone (12fc), (2-(4-(dimethylamino)phenyl)-1H-imidazol-4-yl)(3,4,5-trimethoxyphenyl)methanone (12ga), (2-(4-(dimethylamino)phenyl)-1H-imidazol-4-yl)(4-fluorophenyl)methanone (12gb), (2-(3,4-dimethoxyphenyl)-1H-imidazol-4-yl)(3,4,5-trimethoxyphenyl)methanone (12ha), (2-(4-(benzyloxy)phenyl)-1H-imidazol-4-yl)(4-fluorophenyl)methanone (12jb), (2-(4-bromophenyl)-1H-imidazol-4-yl)(3,4,5-trimethoxyphenyl)methanone (12la), or (2-(4-(trifluoromethyl)phenyl)-1H-imidazol-4-yl)(3,4,5-trimethoxyphenyl)methanone (12pa).

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula XIVa:

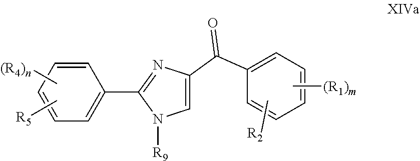

wherein
R₁ and R₄ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₂ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₉ is H, linear or branched alkyl, aryl, CH₂Ph, benzyl, haloalkyl, aminoalkyl, OCH₂Ph, SO₂-Aryl, —(C═O)-Aryl or OH, optionally substituted with at least one of hydrogen, hydroxyl, an aliphatic straight- or branched-chain C₁ to C₁₀ hydrocarbon, alkoxy, haloalkoxy, aryloxy, nitro, cyano, alkyl-CN, halo (e.g., F, Cl, Br, I), haloalkyl, dihaloalkyl, trihaloalkyl, COOH, C(O)Ph, C(O)-alkyl, C(O)O-alkyl, C(O)H, C(O)NH₂, —OC(O)CF₃, OCH₂Ph, amino, aminoalkyl, alkylamino, mesylamino, dialkylamino, arylamino, amido, NHC(O)-alkyl, urea, alkyl-urea, alkylamido (e.g., acetamide), haloalkylamido, arylamido, aryl, and C₅ to C₇ cycloalkyl, arylalkyl, and combinations thereof;
m is an integer between 1-4; and
n is an integer between 1-4;
or a pharmaceutically acceptable salt, hydrate, polymorph, or isomer thereof.

Non limiting examples of CBSI compounds of Formula XIVa are selected from: (4-fluorophenyl)(2-phenyl-1-(phenylsulfonyl)-1H-imidazol-4-yl)methanone (11af), (4-fluorophenyl)(2-(4-methoxyphenyl)-1-(phenylsulfonyl)-1H-imidazol-4-yl)methanone (11cb), (4-fluorophenyl)(1-(phenylsulfonyl)-2-(p-tolyl)-1H-imidazol-4-yl)methanone (11db), (2-(4-chlorophenyl)-1-(phenylsulfonyl)-1H-imidazol-4-yl)(4-fluorophenyl)methanone (11fb), (2-(4-(dimethylamino)phenyl)-1-(phenylsulfonyl)-1H-imidazol-4-yl)(3,4,5-trimethoxyphenyl)methanone (11ga), (2-(4-(dimethylamino)phenyl)-1-(phenylsulfonyl)-1H-imidazol-4-yl)(4-fluorophenyl)methanone (11gb), (2-(3,4-dimethoxyphenyl)-1-(phenylsulfonyl)-1H-imidazol-4-yl)(3,4,5-trimethoxyphenyl)methanone (11ha), (2-(4-(benzyloxy)phenyl)-1-(phenylsulfonyl)-1H-imidazol-4-yl)(4-fluorophenyl)methanone (11jb), (2-(4-(dimethylamino)phenyl)-1-((4-methoxyphenyl)sulfonyl)-1H-imidazol-4-yl)(4-fluorophenyl)methanone (12gba), (1-benzyl-2-(p-tolyl)-1H-imidazol-4-yl)(3,4,5-trimethoxyphenyl)methanone (12daa), (1-methyl-2-(p-tolyl)-1H-imidazol-4-yl)(3,4,5-trimethoxyphenyl)methanone (12dab), or (4-fluorophenyl)(2-(4-methoxyphenyl)-1-methyl-1H-imidazol-4-yl)methanone (12cba).

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula XV:

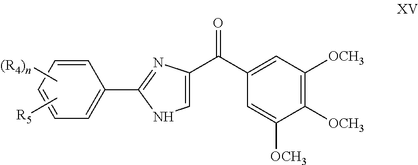

wherein
R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.

Non limiting examples of CBSI compounds of Formula XV are selected from: (2-phenyl-1H-imidazol-4-yl)(3,4,5-trimethoxyphenyl)methanone (12aa), (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (2-(p-tolyl)-1H-imidazol-4-yl)(3,4,5-trimethoxyphenyl)methanone (12da), (3,4,5-trimethoxyphenyl)(2-(3,4,5-trimethoxyphenyl)-1H-imidazol-4-yl)methanone (12ea), (2-(4-chlorophenyl)-1H-imidazol-4-yl)(3,4,5-trimethoxyphenyl)methanone (12fa), (2-(4-(dimethylamino)phenyl)-1H-imidazol-4-yl)(3,4,5-trimethoxyphenyl)methanone (12ga), (2-(3,4-dimethoxyphenyl)-1H-imidazol-4-yl)(3,4,5-trimethoxyphenyl)methanone (12ha), (2-(2-(trifluoromethyl)phenyl)-1H-imidazol-4-yl)(3,4,5-trimethoxyphenyl)methanone (12ia), (2-(4-(benzyloxy)phenyl)-1H-imidazol-4-yl)(3,4,5-trimethoxyphenyl)methanone (12ja), (2-(4-hydroxyphenyl)-1H-imidazol-4-yl)(3,4,5-trimethoxyphenyl)methanone (12ka), (2-(4-bromophenyl)-1H-imidazol-4-yl)(3,4,5-trimethoxyphenyl)methanone (12la), or (2-(4-(trifluoromethyl)phenyl)-1H-imidazol-4-yl)(3,4,5-trimethoxyphenyl)methanone (12pa).

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula XVI:

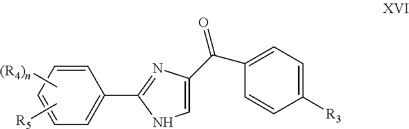

wherein
R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₃ is I, Br, Cl, or F; and
n is 1-4; or a pharmaceutically acceptable salt, hydrate, polymorph, or isomer.

Non limiting examples of CBSI compounds of Formula XVI are selected from: (4-fluorophenyl)(2-phenyl-1H-imidazol-4-yl)methanone (12af), (4-fluorophenyl)(2-(4-methoxyphenyl)-1H-imidazol-4-yl)methanone (12cb), (4-fluorophenyl)(2-(p-tolyl)-1H-imidazol-4-yl)methanone (12db), 4-fluorophenyl)(2-(3,4,5-trimethoxyphenyl)-1H-imidazol-4-yl)methanone (12eb), (2-(4-chlorophenyl)-1H-imidazol-4-yl)(4-fluorophenyl)methanone (12fb), (2-(4-(dimethylamino)phenyl)-1H-imidazol-4-yl)(4-fluorophenyl)methanone (12gb), or (2-(4-(benzyloxy)phenyl)-1H-imidazol-4-yl)(4-fluorophenyl)methanone (12jb).

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula XVII:

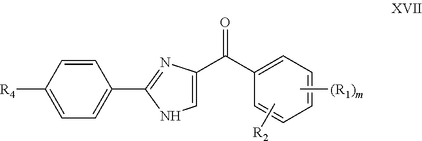

wherein
R₄ is H, O—(C₁-C₄)alkyl, I, Br, Cl, F, (C₁-C₄)alkyl, halo(C₁-C₄)alkyl, amino(C₁-C₄)alkyl, OCH₂Ph, OH, CN, NO₂, —NHCO—(C₁-C₄)alkyl, COOH, C(O)O—(C₁-C₄)alkyl or C(O)H; wherein R₁ and R₂ are independently H, O—(C₁-C₄)alkyl, I, Br, Cl, F, (C₁-C₄)alkyl, halo(C₁-C₄)alkyl, amino(C₁-C₄)alkyl, OCH₂Ph, OH, CN, NO₂, —NHCO—(C₁-C₄)alkyl, COOH, C(O)O—(C₁-C₄)alkyl or C(O)H; and
m is 1-4; or a pharmaceutically acceptable salt, hydrate, polymorph, or isomer thereof.

Non limiting examples of CBSI compounds of Formula XVII are selected from: (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (4-fluorophenyl)(2-(4-methoxyphenyl)-1H-imidazol-4-yl)methanone (12cb), (2-(p-tolyl)-1H-imidazol-4-yl)(3,4,5-trimethoxyphenyl)methanone (12da), (4-fluorophenyl)(2-(p-tolyl)-1H-imidazol-4-yl)methanone (12db), (4-hydroxy-3,5-dimethoxyphenyl)(2-(p-tolyl)-1H-imidazol-4-yl)methanone (12dc), (2-(4-chlorophenyl)-1H-imidazol-4-yl)(3,4,5-trimethoxyphenyl)methanone (12fa), (2-(4-chlorophenyl)-1H-imidazol-4-yl)(4-fluorophenyl)methanone (12fb), (2-(4-chlorophenyl)-1H-imidazol-4-yl)(3,4,5-trihydroxyphenyl)methanone (13fa), (2-(4-(dimethylamino)phenyl)-1H-imidazol-4-yl)(3,4,5-trimethoxyphenyl)methanone (12ga), (2-(4-(dimethylamino)phenyl)-1H-imidazol-4-yl)(4-fluorophenyl)methanone (12gb), (2-(4-(benzyloxy)phenyl)-1H-imidazol-4-yl)(4-fluorophenyl)methanone (12jb), (2-(4-hydroxyphenyl)-1H-imidazol-4-yl)(3,4,5-trimethoxyphenyl)methanone (12ka), (2-(4-bromophenyl)-1H-imidazol-4-yl)(3,4,5-trimethoxyphenyl)methanone (12la), or (2-(4-(trifluoromethyl)phenyl)-1H-imidazol-4-yl)(3,4,5-trimethoxyphenyl)methanone (12pa).

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site

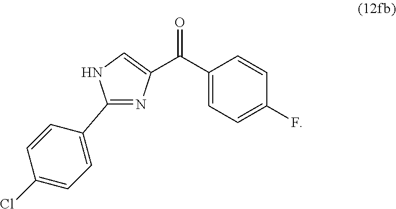 [SMILES: O=C(c1ccc(F)cc1)c1c[nH]c(-c2ccc(Cl)cc2)n1]

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula XVII represented by the structure of formula 12cb:

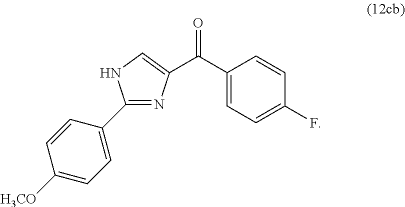 [SMILES: COc1ccc(-c2nc(C(=O)c3ccc(F)cc3)c[nH]2)cc1]

Non limiting examples of CBSI compounds of this invention are selected from: (4-methoxyphenyl)(2-phenyl-1H-imidazol-1-yl)methanone (12aba), (2-phenyl-1H-imidazol-1-yl)(3,4,5-trimethoxyphenyl)methanone (12aaa), 2-phenyl-1-(phenylsulfonyl)-1H-imidazole (10a), 2-(4-nitrophenyl)-1-(phenylsulfonyl)-1H-imidazole (10x), 2-(4-(benzyloxy)phenyl)-1-(phenylsulfonyl)-1H-imidazole (10j), or 2-(2H-indol-5-yl)-4-(3,4,5-trimethoxyphenyl)-1H-imidazo[4,5-c]pyridine (7).

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula XIX:

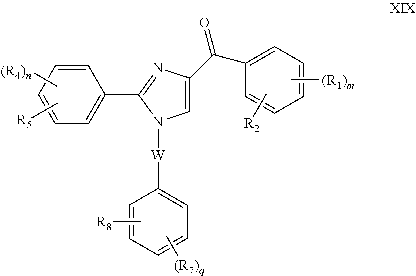

wherein
W is C═O, C═S, SO₂, or S═O;
R₁, R₄ and R₇ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₂, R₅ and R₈ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
m is 1-4;
n is 1-4; and
q is 1-4;
or its pharmaceutically acceptable salt, hydrate, polymorph, or isomer.

Non limiting examples of CBSI compounds of Formula XIX are selected from: (2-(4-(dimethylamino)phenyl)-1-((4-methoxyphenyl)sulfonyl)-1H-imidazol-4-yl)(3,4,5-trimethoxyphenyl)methanone (11gaa); (2-(4-bromophenyl)-1-(phenylsulfonyl)-1H-imidazol-4-yl)(3,4,5-trimethoxyphenyl)methanone (11la), (4-fluorophenyl)(2-(4-methoxyphenyl)-1-(phenylsulfonyl)-1H-imidazol-4-yl)methanone (11cb), (2-(4-chlorophenyl)-1-(phenylsulfonyl)-1H-imidazol-4-yl)(4-fluorophenyl)methanone (11fb), (4-fluorophenyl)(2-phenyl-1-(phenylsulfonyl)-1H-imidazol-4-yl)methanone (11af), (4-fluorophenyl)(1-(phenylsulfonyl)-2-(p-tolyl)-1H-imidazol-4-yl)methanone (11db), (2-(4-(dimethylamino)phenyl)-1-(phenylsulfonyl)-1H-imidazol-4-yl)(3,4,5-trimethoxyphenyl)methanone (11ga), (2-(4-(dimethylamino)phenyl)-1-(phenylsulfonyl)-1H-imidazol-4-yl)(4-fluorophenyl)methanone (11gb), (2-(3,4-dimethoxyphenyl)-1-(phenylsulfonyl)-1H-imidazol-4-yl)(3,4,5-trimethoxyphenyl)methanone (11ha), (2-(4-(benzyloxy)phenyl)-1-(phenylsulfonyl)-1H-imidazol-4-yl)(4-fluorophenyl)methanone (11jb), or (2-(4-(dimethylamino)phenyl)-1-((4-methoxyphenyl)sulfonyl)-1H-imidazol-4-yl)(4-fluorophenyl)methanone (12gba).

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula XIX represented by the structure of formula 11cb:

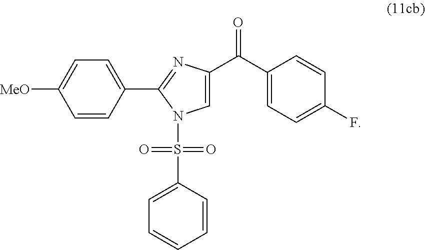 [SMILES: COc1ccc(-c2nc(C(=O)c3ccc(F)cc3)cn2S(=O)(=O)c2ccccc2)cc1]

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula XIX represented by the structure of formula 11fb:

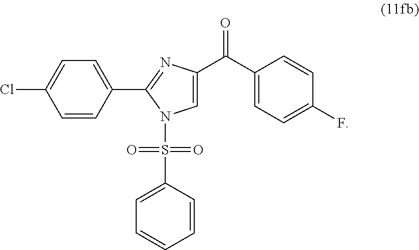 [SMILES: O=C(c1ccc(F)cc1)c1cn(S(=O)(=O)c2ccccc2)c(-c2ccc(Cl)cc2)n1]

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula XX:

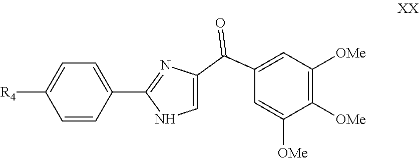

wherein
R₄ is independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer.

Non limiting examples of CBSI compounds of Formula XX are selected from: (2-phenyl-1H-imidazol-4-yl)(3,4,5-trimethoxyphenyl)methanone (12aa), (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (2-(p-tolyl)-1H-imidazol-4-yl)(3,4,5-trimethoxyphenyl)methanone (12da), (2-(4-chlorophenyl)-1H-imidazol-4-yl)(3,4,5-trimethoxyphenyl)methanone (12fa), (2-(4-(dimethylamino)phenyl)-1H-imidazol-4-yl)(3,4,5-trimethoxyphenyl)methanone (12ga), (2-(2-(trifluoromethyl)phenyl)-1H-imidazol-4-yl)(3,4,5-trimethoxyphenyl)methanone (12ia), (2-(4-(benzyloxy)phenyl)-1H-imidazol-4-yl)(3,4,5-trimethoxyphenyl)methanone (12ja), (2-(4-hydroxyphenyl)-1H-imidazol-4-yl)(3,4,5-trimethoxyphenyl)methanone (12ka), (2-(4-bromophenyl)-1H-imidazol-4-yl)(3,4,5-trimethoxyphenyl)methanone (12la), or (2-(4-(trifluoromethyl)phenyl)-1H-imidazol-4-yl)(3,4,5-trimethoxyphenyl)methanone (12pa).

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula XX represented by the structure of formula 12da:

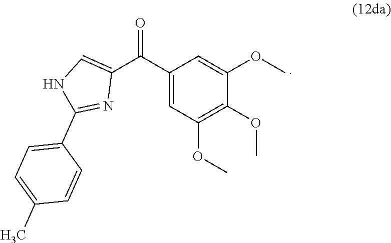 [SMILES: COc1cc(C(=O)c2c[nH]c(-c3ccc(C)cc3)n2)cc(OC)c1OC ; "."]

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula XX represented by the structure of formula 12fa:

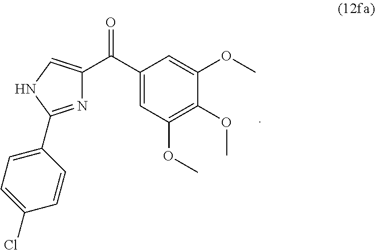 [SMILES: COc1cc(C(=O)c2c[nH]c(-c3ccc(Cl)cc3)n2)cc(OC)c1OC]

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula XXI:

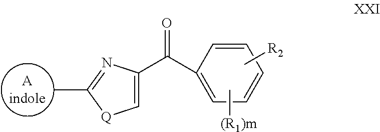

wherein
A is indolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
Q is S or NH;
R₁ and R₂ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
m is 1-4; or a pharmaceutically acceptable salt, hydrate, polymorph, or isomer thereof.

In one embodiment of the method of treating, reducing, inhibiting, slowing the progression of, promoting the regression of, or preventing peripheral artery disease (PAD), A ring of compound of Formula XXI is substituted 5-indolyl. In another embodiment the substitution is —(C═O)-Aryl. In another embodiment, the aryl is 3,4,5-(OCH₃)₃-Ph. In another embodiment, A ring of compound of Formula XXI is 3-indolyl. In another embodiment, A ring of compound of Formula XXI is 5-indolyl. In another embodiment, A ring of compound of Formula XXI is 2-indolyl. Non limiting examples of CBSI compounds of Formula XXI are selected from: (5-(4-(3,4,5-trimethoxybenzoyl)-1H-imidazol-2-yl)-1H-indol-2-yl)(3,4,5-trimethoxyphenyl)methanone (15xaa); (1-(phenylsulfonyl)-2-(1-(phenylsulfonyl)-2-(3,4,5-trimethoxybenzoyl)-1H-indol-5-yl)-1H-imidazol-4-yl)(3,4,5-trimethoxyphenyl)methanone (16xaa); (2-(1H-indol-3-yl)-1H-imidazol-4-yl)(3,4,5-trimethoxyphenyl)methanone (17ya); (2-(1H-indol-2-yl)thiazol-4-yl)(3,4,5-trimethoxyphenyl)methanone (62a); or (2-(1H-indol-5-yl)thiazol-4-yl)(3,4,5-trimethoxyphenyl)methanone (66a).

A particularly preferred method of treating, reducing, inhibiting, slowing the progression of, promoting the regression of, or preventing peripheral artery disease (PAD) uses at least one CBSI compound of Formula XXI including (2-(1H-indol-1-yl)-1H-imidazol-4-yl)(3,4,5-trimethoxyphenyl)methanone; (2-(1H-indol-2-yl)-1H-imidazol-4-yl)(3,4,5-trimethoxyphenyl)methanone; (2-(1H-indol-3-yl)-1H-imidazol-4-yl)(3,4,5-trimethoxyphenyl)methanone (17ya); (2-(1H-indol-4-yl)-1H-imidazol-4-yl)(3,4,5-trimethoxyphenyl)methanone; (2-(1H-indol-5-yl)-1H-imidazol-4-yl)(3,4,5-trimethoxyphenyl)methanone; (2-(1H-indol-6-yl)-1H-imidazol-4-yl)(3,4,5-trimethoxyphenyl)methanone; or (2-(1H-indol-7-yl)-1H-imidazol-4-yl)(3,4,5-trimethoxyphenyl)methanone.

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula XXIa:

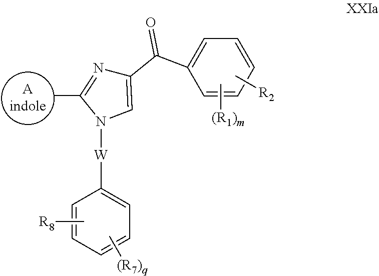

wherein
W is C═O, C═S, SO₂, or S═O;
A is indolyl optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₁ and R₂ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
R₇ and R₈ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
m is 1-4; and
q is 1-4; or a pharmaceutically acceptable salt, hydrate, polymorph, or isomer thereof.

Non limiting examples of CBSI compounds of Formula XXIa are selected from: (1-(phenylsulfonyl)-2-(1-(phenylsulfonyl)-2-(3,4,5-trimethoxybenzoyl)-1H-indol-5-yl)-1H-imidazol-4-yl)(3,4,5-trimethoxyphenyl)methanone (16xaa); or (1-(phenylsulfonyl)-2-(1-(phenylsulfonyl)-1H-indol-3-yl)-1H-imidazol-4-yl)(3,4,5-trimethoxyphenyl)methanone (17yaa).

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula XXII:

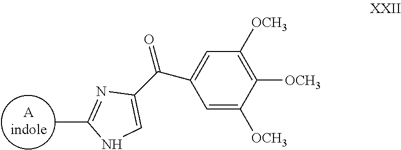

wherein
A is indolyl optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
or a pharmaceutically acceptable salt, hydrate, polymorph, or isomer thereof.

In one embodiment of the method of treating, reducing, inhibiting, slowing the progression of, promoting the regression of, or preventing peripheral artery disease (PAD), A ring of compound of Formula XXII is substituted 5-indolyl. In another embodiment the substitution is —(C═O)-Aryl. In another embodiment, the aryl is 3,4,5-(OCH₃)₃-Ph. In another embodiment, A ring of compound of Formula XXII is 3-indolyl. Non limiting examples of CBSI compounds of Formula XXII are selected from: (5-(4-(3,4,5-trimethoxybenzoyl)-1H-imidazol-2-yl)-1H-indol-2-yl)(3,4,5-trimethoxyphenyl)methanone (15xaa); and (2-(1H-indol-3-yl)-1H-imidazol-4-yl)(3,4,5-trimethoxyphenyl)methanone (17ya).

The invention also encompasses methods of treating, reducing, inhibiting, slowing the progression of, promoting the regression of, or preventing peripheral artery disease (PAD) in a subject in need thereof by administering to the subject a formulation having a therapeutically effective amount of a colchicine binding site inhibitor (CBSI) compound of the Formula XXI or XXII represented by the structure of Formula 17ya:

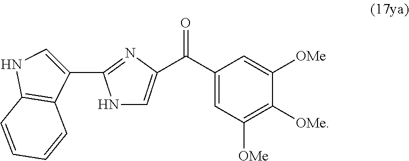 [SMILES: COc1cc(C(=O)c2c[nH]c(-c3c[nH]c4ccccc34)n2)cc(OC)c1OC]

In one embodiment of the method, R₄ and R₅ of compounds of formula XIII-XVI are hydrogens. Non-limiting examples of CBSI compounds of Formula XIII-XVI wherein R₄ and R₅ are hydrogens are selected from (2-phenyl-1H-imidazol-4-yl)(3,4,5-trimethoxyphenyl)methanone (12aa); (4-methoxyphenyl)(2-phenyl-1H-imidazol-4-yl)methanone (12ab); (3-methoxyphenyl)(2-phenyl-1H-imidazol-4-yl)methanone (12ac); (3,5-dimethoxyphenyl)(2-phenyl-1H-imidazol-4-yl)methanone (12ad); (3,4-dimethoxyphenyl)(2-phenyl-1H-imidazol-4-yl)methanone (12ae); (4-fluorophenyl)(2-phenyl-1H-imidazol-4-yl)methanone (12af); (3-fluorophenyl)(2-phenyl-1H-imidazol-4-yl)methanone (12ag); (2-phenyl-1H-imidazol-4-yl)(p-tolyl)methanone (12ah); or (2-phenyl-1H-imidazol-4-yl)(m-tolyl)methanone (12ai).

### Pharmacotherapy in ASCVD

Statins are the cornerstone of the prevention and treatment of atherosclerotic cardiovascular disease (ASCVD), including the ASCVD subtypes of CAD and PAD, and a component of almost any optimal medical therapy. Also known as HMG-CoA reductase inhibitors, statins are a class of prescription drugs that reduce LDL (low-density lipoprotein) cholesterol (LDL-C), or “bad cholesterol” by blocking its synthesis in the body. They can also increase HDL (high-density lipoprotein) concentrations and lower triglyceride levels. For decades, statins have been used as the primary pharmacotherapy in hyperlipidemia as primary prevention or in combination with other classes of agents as secondary prevention in the context of ASCVD. Recent guidelines have expanded the patient population where statins are indicated, resulting in an overall increase in the use of statins by approximately 40-fold between 1991 and 2022. Further, in order to reduce major adverse cardiovascular events (MACE), intensive statin therapy has been recently popularized wherein the goals of statin therapy are driven by achievement of at least a pre-specified amount of LDL-C reduction. Correspondingly, increased statin doses also increase the liabilities for statin toxicities and drug-drug interactions with PGP or CYP inhibitors or inducers, as discussed in greater detail herein. Notably, co-administering with colchicine has elevated muscle-related toxicity risk.

However, even under intensive statin therapy, a significant residual ASCVD risk remains. Accordingly, other classes of lipid-lowering agents have been approved including the proprotein convertase subtilisin/kexin type 9 (PCSK-9) inhibitors. These drugs lower LDL cholesterol and can be taken alone or in addition to a statin. Examples of monoclonal antibody PCSK9 inhibitors include: alirocumab (Praluent) and evolocumab (Repatha). Inclisiran (Leqvio), a small interfering RNA that targets PCSK9, shows comparable effects to that of PCSK9 monoclonal antibodies. Ezetimibe (Zetia) is distinct from these PCSK9 and statin agents because it does not inhibit cholesterol synthesis in the liver or increase bile acid excretion. It belongs to a class of lipid-lowering compounds that selectively inhibits the intestinal absorption of cholesterol and related phytosterols. This drug is an add-on therapy to other lipid-lowering agents in certain high-risk adults. Fish oil products containing omega-3 fatty acids, such as eicosapentaenoic acid (EPA) and docosahexaenoic acid (DHA), can reduce triglycerides in patients with severe hypertriglyceridemia. Novel and emerging lipid-lowering agents include bempedoic acid (an ATP citrate lyase inhibitor), which is a valuable treatment option for patients with statin intolerance. Also, high dose icosapent ethyl (a modified eicosapentaenoic acid preparation) shows cardiovascular benefit and vinacumab (an angiopoietin-like 3 (ANGPTL3) monoclonal antibody) reduces plasma LDL-C levels in patients with refractory hypercholesterolemia. Despite many approved agents of several classes, there remains an unmet clinical need for novel lipid-lowering agents that can target low-density lipoprotein cholesterol (LDL-C) and other atherogenic particles, and also a need to target novel non-lipid-lowering atherosclerotic mechanisms of action.

Anti-platelet agents are another cornerstone used in the optimal medical therapy of ASCVD. Anti-platelet therapy is primarily used to prevent thrombotic events by inhibiting platelet aggregation, and is typically administered as low-dose aspirin (irreversible inhibitor of cyclooxygenase-1 (COX-1) enzyme present in the platelets), either alone or in combination with other antiplatelet medications like clopidogrel, prasugrel, or ticagrelor (adenosine diphosphate P2Y₁₂ inhibitors), depending on the patient's specific situation and risk factors. This approach is particularly important in secondary prevention (after a cardiovascular event) where it aims to reduce the risk of future MACE like heart attacks and strokes. The third group of antiplatelet agents available for clinical use are the glycoprotein IIb/IIIa inhibitors abciximab, tirofiban and eptifibatide. They are administered intravenously and target the final pathway of platelet aggregation. Another class of antiplatelet agent is the orally active phosphodiesterase inhibitors, represented by dipyridamole. Lastly, vorapaxar is an orally active thrombin receptor inhibitor that prevents thrombin from activating platelets via the protease-activated receptor (PAR)-1.

Other anti-coagulants, in addition to anti-platelet agents, are sometimes used in ASCVD in certain high-risk patients. These additional anti-coagulants are typically direct oral anticoagulants (DOACs) such as rivaroxaban (Xarelto; used in addition to antiplatelet agents in patients with atrial fibrillation or recent stent placement), dabigatran (Pradaxa; requires careful monitoring due to potential interactions with other medications), abixaban, endoxaban, and betrixaban (prophylactic agent in mechanical prosthetic valve, or moderate to severe mitral stenosis). Similar to statins, the DOAC agents are typically a liability for drug-drug interactions with PGP inhibitors and CYP3A4 inhibitors. An older class of anti-coagulants are the vitamin K antagonists (VKA) such as warfarin, acenocoumarol, and phenprocoumon. The VKA class has largely been replaced in ASCVD by the DOACs due to their relatively narrow therapeutic window and increased risk of bleeding. Warfarin is a liability for drug-drug interactions with predominantly CYP2C9 inducers or inhibitors, but also CYP3A4 or CYP2A1 inducers or inhibitors.

Optimal medical therapy in most subtypes of ASCVD including PAD and CAD is usually a combination of lipid-lowering therapies (most commonly at least statins) and anti-platelet therapies. Occasionally, other classes of agents are also used in high-risk patients, or when needed due to co-morbidities such as anti-hypertensive agents if blood pressure is high or anti-diabetic agents if blood sugar is high or in the presence of heart failure or renal disease. Anti-hypertensives are more commonly used in CAD than other types of ASCVD. Classes of anti-hypertensives used in CAD include beta blockers such as acebutolol, atenolol, bisoprolol, carvedilol, esmolol, labetalol, metoprolol, nadolol, nebivolol, propranolol, sotalol, or timolol which are used first-line in CAD, or sometimes calcium channel blockers (if beta blockers are ineffective or contraindicated) such as amlodipine, bepridil, diltiazem, felodipine, nicardipine, nifedipine, nisoldipine, or verapamil. Often a beta-blocker or a calcium channel blocker is combined with an angiotensin-converting enzyme (ACE) inhibitors such as ramipril, captopril, enalapril, lisinopril, perindopril, or trandolapril, or an angiotensin 2 receptor blockers (ARBs) such as candesartan, losartan, or valsartan. This combination therapy can be used, if needed, to lower blood pressure, to avoid accelerating the atherosclerotic process, and to relieve the workload on the heart. In some ASCVD disease, thiazide diuretics are prescribed such as hydrochlorothiazide, chlorthalidone, indapamide, metolazone, bendroflumethiazide, or chlorothiazide.

While some anti-hypertensive agents such beta-blockers and calcium channel blockers provide relief from angina (chest pain), other anti-anginal agents include nitroglycerin (widens the heart arteries to reduce chest pain) or ranolazine.

Classes of anti-diabetic agents most commonly used in ASCVD include glucagon-like peptide-1 receptor agonists (GLP-1 RA) such as liraglutide, semaglutide, dulaglutide, and albiglutide, and sodium-glucose cotransporter 2 inhibitors (SGLT2i) such as empaglifozin, canaglifozin, dapaglifozin, and sotaglifozin. Both SGLT2i and GLP-1 RA agents have been shown to reduce the risk of major cardiovascular events (MACE) like myocardial infarction, stroke, and cardiovascular death in clinical trials, making them particularly beneficial for individuals with established ASCVD. Many of the GLP-1 RA and SGLT2i agents in addition to their glucose lowering effects (measured as decreased HbAlc) also have other positive effects such as weight loss, blood pressure reduction, and renal protective properties that would be beneficial in ASCVD. For GLP-1 RA agents, some preclinical evidence suggests direct anti-atherosclerotic effects as well, mediated by anti-inflammation (Rakipovski, G et al. JACC Basic. Trans. Sci. 3, 844-857, 2018). For SGLT2i agents, there is also good clinical evidence for improved ejection fraction and overall benefit in heart failure patients.

### Adverse Events Due Colchicine Toxicity

Colchicine is a natural alkaloid that has anti-inflammatory properties that can be therapeutic for a variety of diseases when used at low doses; whereas high doses are generally cytotoxic, especially in rapidly dividing cells. Approved colchicine doses include 0.5 mg daily for atherosclerotic cardiovascular disease (ASCVD; approved 2023), 0.6 mg daily for prophylaxis of gout or 1.2 mg initial dose plus 0.6 mg one hour later for gout flares (approved 2009), and 1.2 to 2.4 mg daily for familial Mediterranean fever (approved 2009). Acute oral ingestion exceeding 0.5-0.8 mg/kg of colchicine is considered to be fatal, or alternatively colchicine can accumulate in certain organs during chronic therapy. Nevertheless, there is no clear-cut line between nontoxic, toxic, and lethal doses of colchicine in human. This narrow therapeutic window is exacerbated by unstable pharmacokinetics based on inter-individual variation in CYP3A4 and PGP expression and the presence of inducers/inhibitors of CYP3A4 and/or PGP. Long-term use of colchicine such as for ASCVD increases the risk of chronic colchicine poisoning due to accumulation or drug-drug interaction. These uncertainties cause colchicine to have a wide range of half-lives in humans of 9.3-30 hours. Correspondingly, in some cases, there is no clear delimitation between therapeutic doses and toxic doses, and colchicine poisoning is common. Colchicine at high doses is cytotoxic, especially in tissues with high cell turnover, owing to its mechanism of action of disrupting microtubule formation which is essential for cell division and function. Unfortunately, there is no known antidote and no established medical treatment for systemic colchicine toxicity. Further, symptoms of systemic colchicine toxicity have a delayed onset of 12 hours. Colchicine adverse events can be thought of as a result of colchicine accumulation to cytotoxicity in certain organs including the intestinal mucosa, heart, muscles, kidneys, liver, lungs, bone marrow, and brain. The accumulation is apparent in the increase in volume of distribution for colchicine from 7-10 L/kg at therapeutic doses to 21 L/kg in colchicine toxicity. In overview, colchicine toxicity causes various metabolic disturbances and can cause multiorgan failure and death.

Expected adverse events of colchicine (26-77%) include gastrointestinal (GI) symptoms such as diarrhea, nausea, cramping, abdominal pain, vomiting, etc. which can be tolerable, but these intestinal mucosa cytotoxicities are also early symptoms of systemic colchicine toxicity (accumulation in GI). Multiple organ dysfunction can occur and each organ will be discussed individually below as if colchicine accumulated to, or was overdosed to, cellular cytotoxicity (anti-mitosis) in the organ. In the muscles, colchicine can cause adverse events due to myotoxicities including acute myocardial injury (heart attack) which can cause chest pain, arrhythmias or cardiac failure. Skeletal muscle adverse events include muscle pain, weakness, or stiffness, elevations in creatine phosphokinase (CPK), severe myopathy, and rhabdomyolysis (muscle breakdown). Indirectly, acute kidney injury (AKI) secondary to the rhabdomyolysis can be a result of myotoxicity and can be observed as dark red or cola-colored urine, or decreased urine output. Myoglobin is released as muscle degrades and this protein precipitates in the renal tubules causing potentially life-threatening renal failure. Cumulative or even synergistic rhabdomyolysis risk is seen with co-therapy with other drugs causing rhabdomyolysis such as all statin drugs (class effect), fibrates (like gemfibrozil, bezafibrate), and high dose salicylates (aspirin). Statins and anti-platelet agents (most commonly aspirin) are the cornerstone of ASCVD therapy, so rhabdomyolysis incidence due to co-therapy with colchicine is exacerbated by drug-drug interactions between colchicine and statins and/or aspirin. But rhabdomyolysis may also be exacerbated by other co-therapies with either colchicine (drug-drug interactions with CYP3A4, PGP inhibitors) or statins (drug-drug interactions with CYP3A4, PGP, CYP2C9, or CYP2C19 inhibitors). Adverse events such as AKI and liver damage (elevated liver enzymes, jaundice, etc.) is also seen in colchicine toxicity, and pre-existing renal and liver impairment increases the risk to these organs. Adverse events related to liver damage can include coagulation disorders such as disseminated intravascular coagulation. Adverse events due to acute lung injury is also seen, possibly exacerbated by respiratory muscle (diaphragm) weakness (myotoxicity), with symptoms of coughing, shortness of breath, cyanosis, respiratory distress or failure, etc. Adverse events include blood dyscrasias, e.g. due to bone marrow suppression (hematopoetic cell cytotoxicity) can be seen as pancytopenia, or erythro-, leuco-, lympho-, neutro-, or thrombocytopenias starting at 3-5 days and lasting a week, that can increase the risk of infections, bleeding, and anemia. Neurotoxicity is another adverse event of colchicine which is a result of neuronal toxicity in certain regions of the brain, and can result in seizures, CNS symptoms (confusion, amnesia, numbness, etc.), and worsening of myotoxicity (neuromuscular toxicity); neurotoxicity is reversible in mild cases.

In overview, colchicine toxicity can occur in three phases: phase 1 occurs within 24 hours of the dose, and characterized by GI symptoms and hypovolemia; phase 2 occurs 1 to 7 days after the dose and marked by multiorgan failure, including respiratory failure, cardiac arrhythmias and arrest, encephalopathy, seizures, renal failure, liver failure, disseminated intravascular coagulation, bone marrow suppression, pancytopenia, hemolysis, metabolic derangements, myopathy, neuropathy, and secondary sepsis; and phase 3 occurs one to three weeks after ingestion and can be characterized by general recovery, and alopecia.

Patients with low apparent levels of PGP or CYP3A4 may have increased risk, but screening for this is not routine (or maybe not even possible). In renal or hepatic impairment, co-therapy with strong inhibitors of PGP or CYP3A4, such as amiodarone, diltiazem, grapefruit juice, atorvastatin, lovastatin, ranolazine, simvastatin, and verapamil, is contraindicated; whereas in the absence of renal or hepatic impairment, colchicine dose reductions are recommended in strong PGP or CYP3A4 inhibitor co-therapy. The full list of possible CYP3A4 and PGP inhibitors is quite extensive and would be known to the skilled artisan, whereas listed above are just a few cardiovascular related agents commonly co-administered in ASCVD. In summary, co-morbid conditions and co-pharmacotherapies further blur the line between therapeutic and toxic colchicine doses.

In one embodiment of the method, the compounds of this invention are the pure (E)-isomers. In another embodiment, the compounds of this invention are the pure (Z)-isomers. In another embodiment, the compounds of this invention are a mixture of the (E) and the (Z) isomers. In one embodiment, the compounds of this invention are the pure (R)-isomers. In another embodiment, the compounds of this invention are the pure (S)-isomers. In another embodiment, the compounds of this invention are a mixture of the (R) and the (S) isomers.

The compounds of the present invention can also be present in the form of a racemic mixture, containing substantially equivalent amounts of stereoisomers. In another embodiment, the compounds of the present invention can be prepared or otherwise isolated, using known procedures, to obtain a stereoisomer substantially free of its corresponding stereoisomer (i.e., substantially pure). As used herein, the term “substantially pure” refers to stereoisomer that is at least about 95% pure in one isomer. Alternatively, the stereoisomer purity may be at least about 98% pure, and more preferably at least about 99% pure.

Compounds can also be in the form of a hydrate, which means that the compound further includes a stoichiometric or non-stoichiometric amount of water bound by non-covalent intermolecular forces.

The invention includes “pharmaceutically acceptable salts” of the compounds used in the method of the invention, which may be produced, by reaction of a compound of this invention with an acid or base. Certain compounds, particularly those possessing acid or basic groups, can also be in the form of a salt, preferably a pharmaceutically acceptable salt. As used herein, the term “pharmaceutically acceptable salt” refers to those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxylic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

Suitable pharmaceutically acceptable salts of amines of compounds used in the method of the invention may be prepared from an inorganic acid or from an organic acid. In one embodiment, examples of inorganic salts of amines are bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphate, sulfates, sulfamates, sulfanilates, sulfonic acids (alkylsulfonates, arylsulfonates, halogen substituted alkylsulfonates, halogen substituted arylsulfonates), sulfonates and thiocyanates.

Examples of organic salts of amines include, but are not limited to, aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates gluconates, glutamates, glycolates, glucorate, glucoheptanoates, glycerophosphates, gluceptates, glycollylarsanilates, glutarates, glutamate, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, nitrates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartrates, theophyllineacetates, p-toluenesulfonates (tosylates), trifluoroacetates, terephthalates, tannates, teoclates, trihaloacetates, triethiodide, tricarboxylates, undecanoates and valerates.

Examples of inorganic salts of carboxylic acids or hydroxyls may be selected from ammonium, alkali metals to include lithium, sodium, potassium, cesium; alkaline earth metals to include calcium, magnesium, aluminium; zinc, barium, cholines, quaternary ammoniums.

Examples of organic salts of carboxylic acids or hydroxyl may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglamines, N-methyl-D-glucamines, N,N′-dibenzylethylenediamines, nicotinamides, organic amines, ornithines, pyridines, picolies, piperazines, procain, tris(hydroxymethyl)methylamines, triethylamines, triethanolamines, trimethylamines, tromethamines and ureas.

Typical salts include, but are not limited to, hydrofluoric, hydrochloric, hydrobromic, hydroiodic, boric, nitric, perchloric, phosphoric, sulfuric, acetate, citrate, maleate, malate, or mesylate. Preferred salts include hydrofluoric, hydrochloric, hydrobromic, hydroiodic, acetate, citrate, maleate, or mesylate. More preferred salts include hydrochloric, acetate, or maleate.

The salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of an existing salt for another ion or suitable ion-exchange resin.

The compounds used in the methods of the invention were synthesized using the methodology described in U.S. Pat. Nos. 8,592,465; 8,822,513; 9,029,408; 9,334,242; 9,447,049; 10,301,285; and 11,084,811, hereby incorporated by reference.

### Pharmaceutical Composition

The methods of the invention include the administration of a pharmaceutical composition including a pharmaceutically acceptable carrier and at least one compound described herein. Typically, the pharmaceutical composition may include a compound or its pharmaceutically acceptable salt, and at least one pharmaceutically acceptable excipient. The term “pharmaceutically acceptable excipient” refers to any suitable adjuvants, carriers, excipients, flavorant, or stabilizers, and can be used in pharmaceutical formulations either in solid or liquid form. Such forms include, but are not limited to, tablets, capsules, powders, solutions, suspensions, or emulsions.

The amount of compound used in the method and the dosage regimen for treating a disease condition depends on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods.

Typically, the formulations have from about 0.01 to about 99 percent by weight of at least one compound by weight, preferably from about 20 to 75 percent of active compound(s), together with the adjuvants, carriers and/or excipients. While individual needs may vary, determination of optimal ranges of effective amounts of each component is within the skill of the art. Typical daily dosages include about 0.1 mg to about 100 mg or about 0.2 mg to about 20 mg, preferred daily dosages include about 1 mg to about 10 mg, and the most preferred daily dosages include about 3 mg to about 9 mg of the compound. Other preferred dosages include the anti-atherosclerotic cardiovascular disease compound of the invention in an amount of about 4 mg to about 45 mg, or 9 mg to about 18 mg. Alternatively, a dose is from about 0.01 to 150 mg/kg body weight, preferably from about 1 mg to about 100 mg/kg body weight, and more preferably from about 2 to 50 mg/kg body weight, may be appropriate. The daily dose can be administered in one to four doses per day. Preferably, the total daily dose is divided and administered twice daily. E.g., about 1.5 mg twice daily or about 4.5 mg twice daily. Treatment regimen for the administration of the compounds of the present invention can also be determined readily by those with ordinary skill in art. That is, the frequency of administration and size of the dose can be established by routine optimization, preferably while minimizing any side effects.

Lower or higher doses than those recited above may be required. Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a subject's condition, a maintenance dose of a compound, composition or formulation may be administered, if necessary. Subsequently, the dosage or frequency of administration, or both, may be reduced, as a function of the symptoms, to a level at which the improved condition is retained when the symptoms have been alleviated to the desired level. Subjects may, however, require intermittent treatment on a long-term basis upon any recurrence of disease symptoms.

The solid unit dosage forms can be of the conventional type. The solid form can be a capsule and the like, such as an ordinary gelatin type containing the compounds and a carrier. Carriers include, but are not limited to, lubricants and inert fillers such as, castor oil and similar materials, lactose, sucrose, or cornstarch. The formulations may be tabulated with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The tablets, capsules, and the like can also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it can contain, in addition to materials of the above type, a liquid carrier such as a fatty oil.

The invention can be mixed at cold temperatures, room temperature, or elevated temperatures with a liquid carrier such as a fatty oil, castor oil, or other similar oil to manufacture tablets, capsules, and the like.

Various other materials may be present as coatings or to modify the physical form of the dosage unit. For instance, tablets can be coated with shellac, sugar, or both. A syrup can contain, in addition to active ingredient, sucrose as a sweetening agent, methyl and propylparabens as preservatives, a dye, and flavoring such as cherry or orange flavor.

For oral therapeutic administration, the formulation may include excipients and used in the form of tablets, capsules, elixirs, suspensions, syrups, and the like. Such compositions and preparations should contain at least 0.1% of active compound. The percentage of the compound in these compositions can, of course, be varied and can conveniently be between about 2% to about 60% of the weight of the unit. The amount of active compound in such therapeutically useful compositions is such that a suitable dosage will be obtained. Typical compositions according to the present invention are prepared so that an oral dosage unit contains between about 0.1 mg and 100 mg of active compound, and preferred oral compositions contain between 1 mg and 10 mg of active compound.

The formulations may be orally administered with an inert diluent, or with an assimilable edible carrier, or they can be enclosed in hard or soft shell capsules, or they can be compressed into tablets, or they can be incorporated directly with the food of the diet. A preferred formulation is an oral formulation.

The pharmaceutical forms suitable for injectable use include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds or pharmaceutical compositions used in the method of the present invention may also be administered in injectable dosages by solution or suspension of these materials in a physiologically acceptable diluent with a pharmaceutical adjuvant, carrier or excipient. Such adjuvants, carriers and/or excipients include, but are not limited to, sterile liquids, such as water and oils, with or without the addition of a surfactant and other pharmaceutically and physiologically acceptable components. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

The formulation may also be administered parenterally. Solutions or suspensions of these formulations can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

For use as aerosols, the formulations may be in solution or suspension may be packaged in a pressurized aerosol container together with suitable propellants, for example, hydrocarbon propellants like propane, butane, or isobutane with conventional adjuvants. The formulations also may be administered in a non-pressurized form such as in a nebulizer or atomizer.

When administering the formulations in the methods of the invention, the formulations may be administered systemically or sequentially. Administration can be accomplished in any manner effective for delivering the compounds or the pharmaceutical compositions to the site of the plaque. Exemplary modes of administration include, without limitation, administering the compounds or compositions orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes.

The methods may include co-therapy comprised of a CBSI compound of the invention and other drugs used for ASCVD or its subtypes of CAD or PAD. Other drugs used for ASCVD or its subtypes of CAD or PAD can include many possible combinations of agents used in ASCVD or its subtypes CAD or PAD, as discussed herein. Other drugs used for ASCVD, CAD or PAD typically comprises at least one lipid-lowering agent and at least one anti-platelet agent as the cornerstone of medical therapy. Other drugs may additionally comprise any or all of several other classes of pharmacotherapy including other anti-coagulants (not just anti-platelet agents), anti-hypertensives, anti-anginals, and anti-diabetics; as will be determined to be appropriate given the specific subject to be treated and their risk factors and co-morbidities. In some embodiments, other drugs comprise at least one of said lipid-lowering agents selected from such classes as statins, PCSK-9 inhibitors, cholesterol intestinal absorption blockers, fish oil products, ATP citrate lyase inhibitors, modified eicosapentaenoic acid preparations, and angiopoietin-like 3 (ANGPTL3) monoclonal antibodies. In many embodiments, said lipid-lowering agent comprises a statin as the agent of choice. In some embodiments, the lipid-lowering agent may additionally or alternatively comprise a non-statin agent as described herein. Accordingly, the methods may comprise co-therapy of a CBSI compound of the invention with a statin (HMG-CoA reductase inhibitors) such as atorvastatin, fluvastatin, lovastatin, lovastatin extended-release, pitavastatin, pravastatin, rosuvastatin, simvastatin, and the like. However, even under intensive statin therapy, a significant residual ASCVD risk remains. Accordingly, other classes of lipid-lowering agents have been approved including the proprotein convertase subtilisin/kexin type 9 (PCSK-9) inhibitors that can be taken alone or in addition to a statin. Accordingly, the methods may comprise co-therapy of a CBSI compound of the invention with a PCSK9 inhibitor such as alirocumab (Praluent), evolocumab (Repatha), inclisiran (Leqvio), and the like. The methods of the invention may comprise co-therapy of a CBSI compound of the invention with further lipid-lowering agents such as ezetimibe (Zetia) and the like, that selectively inhibit the intestinal absorption of cholesterol and related phytosterols; or fish oil products containing omega-3 fatty acids, such as eicosapentaenoic acid (EPA) and docosahexaenoic acid (DHA), and the like, that can reduce triglycerides in patients with severe hypertriglyceridemia. The methods of the invention may comprise co-therapy of a CBSI compound of the invention with novel and an emerging lipid-lowering agent such as ATP citrate lyase inhibitors such as bempedoic acid (an option available for statin intolerance) and the like; or a modified eicosapentaenoic acid preparation such as high dose icosapent ethyl and the like; or angiopoietin-like 3 (ANGPTL3) monoclonal antibodies such as vinacumab and the like.

As stated above, anti-platelet agents are another cornerstone used in the optimal medical therapy of ASCVD and its subtypes CAD and PAD. Accordingly, other drugs used commonly comprise lipid-lowering and anti-platelet agents. The methods of the invention may comprise co-therapy of a CBSI compound of the invention with anti-platelet therapies such as an irreversible inhibitor of cyclooxygenase-1 (COX-1; enzyme present in the platelets) such as acetylated salicylates such as low-dose aspirin and the like; either alone or in combination with other antiplatelet medications such as adenosine diphosphate P2Y₁₂ inhibitors such as clopidogrel, prasugrel, or ticagrelor, and the like. The methods of the invention may comprise co-therapy of a CBSI compound of the invention with a glycoprotein IIb/IIIa inhibitor such as abciximab, tirofiban, eptifibatide, and the like. The methods of the invention may comprise co-therapy of a CBSI compound of the invention with an orally active phosphodiesterase inhibitors such as dipyridamole, and the like. The methods of the invention may comprise co-therapy of a CBSI compound of the invention with an orally active thrombin receptor inhibitor such as vorapaxar, and the like. At a minimum, other drugs typically comprise a statin and low-dose aspirin. However, the prescriber may administer other drugs that involve other possible combinations of the specific lipid-lowering agents and anti-platelet agents, as discussed herein. Accordingly, the methods may comprise co-therapy of a CBSI compound of the invention with many possible pharmacotherapeutic combinations. Accordingly, other drugs used in ASCVD, CAD or PAD typically comprise lipid-lowering agents and anti-platelet agents and optionally comprise other agents of several classes, as described.

Other anti-coagulants, in addition to anti-platelet agents, are sometimes used in ASCVD in certain high-risk subjects. The methods of the invention may comprise co-therapy of a CBSI compound of the invention with a direct oral anticoagulant (DOAC) such as rivaroxaban, dabigatran, abixaban, endoxaban, betrixaban, and the like. Similar to statins, the DOACs are typically susceptible to drug-drug interactions with PGP inhibitors and CYP3A4 inhibitors. In certain embodiments, at least one of ASCVD, CAD, or PAD can be treated with a CBSI compound of the invention with reduced risk of, or without the risk of, adverse events associated with colchicine such as diarrhea, vomiting, abdominal cramps, neutropenia, blood dyscrasias, myalgia, rhabdomyolysis, or neurotoxicity. In certain embodiments, at least one of ASCVD, CAD, or PAD can be treated with a CBSI compound of the invention with reduced risk of, or without the risk of, adverse events associated with colchicine such as diarrhea, nausea, vomiting, cramping, abdominal pain, and hypovolemia. In certain embodiments, at least one of ASCVD, CAD, or PAD can be treated with a CBSI compound of the invention with reduced risk of, or without the risk of, adverse events associated with colchicine such as acute myocardial injury, chest pain, arrhythmias, cardiac arrest, or cardiac failure. In certain embodiments, at least one of ASCVD, CAD, or PAD can be treated with a CBSI compound of the invention with reduced risk of, or without the risk of, adverse events associated with colchicine such as myotoxicities, muscle pain, muscle weakness, muscle stiffness, elevated creatine phosphotransferase (CPK), myopathies, and rhabdomyolysis. In certain embodiments, at least one of ASCVD, CAD, or PAD can be treated with a CBSI compound of the invention with reduced risk of, or without the risk of, adverse events associated with colchicine such as acute kidney injury, hematuria, rhabdomyolysis, dark red or cola-colored urine, and decreased urinary output. In certain embodiments, at least one of ASCVD, CAD, or PAD can be treated with a CBSI compound of the invention with reduced risk of, or without the risk of, adverse events associated with colchicine such as acute liver injury, elevated liver enzymes, jaundice, coagulation disorders, disseminated intravascular coagulation, etc. In certain embodiments, at least one of ASCVD, CAD, or PAD can be treated with a CBSI compound of the invention with reduced risk of, or without the risk of, adverse events associated with colchicine such as acute lung injury, coughing, shortness of breath, cyanosis, respiratory distress, and respiratory failure. In certain embodiments, at least one of ASCVD, CAD, or PAD can be treated with a CBSI compound of the invention with reduced risk of, or without the risk of, adverse events associated with colchicine such as blood dyscrasias, bone marrow suppression, pancytopenia, erythrocytopenia, leucopenia, lymphopenia, neutropenia, thrombocytopenia, infections, hemolysis, and sepsis. In certain embodiments, at least one of ASCVD, CAD, or PAD can be treated with a CBSI compound of the invention with reduced risk of, or without the risk of, adverse events associated with colchicine such as neurotoxicity, encephalopathy, brain damage, seizures, central nervous system symptoms, confusion, amnesia, neuromuscular toxicity, worsening myotoxicity, and neuropathy. In certain embodiments, at least one of ASCVD, CAD, or PAD can be treated with a CBSI compound of the invention with reduced risk of, or without the risk of, adverse events associated with colchicine such as alopecia.

In certain embodiments, at least one of ASCVD, CAD, or PAD can be treated with a CBSI compound of the invention with reduced risk of, or without the risk of, drug-drug interactions between colchicine and DOACs. An older class of anti-coagulants are the vitamin K antagonists (VKA). The methods of the invention may comprise co-therapy of a CBSI compound of the invention with a VKA such as warfarin, acenocoumarol, phenprocoumon, and the like. The VKA class has largely been replaced in ASCVD by the DOACs due to the relatively narrow therapeutic window and increased risk of bleeding for VKA agents. In certain embodiments, at least one of ASCVD, CAD, or PAD can be treated with a CBSI compound of the invention with reduced risk of, or without the risk of, adverse events associated with colchicine. In certain embodiments, at least one of ASCVD, CAD, or PAD can be treated with a CBSI compound of the invention with reduced risk of, or without the risk of, drug-drug interactions between colchicine and VKA. In one embodiment, the VKA is warfarin.

As mentioned previously, typical pharmacotherapy in most subtypes of ASCVD including PAD and CAD is a combination of lipid-lowering therapies (most commonly statins) and anti-platelet therapies. Occasionally, other classes of agents are also used in high-risk patients, or when needed due to co-morbidities. Examples include anti-hypertensive agents if blood pressure is high and/or anti-diabetic agents if blood sugar is high or in the presence of heart failure or renal disease. In some embodiments, anti-hypertensive agents are more commonly used to treat CAD. The methods of the invention may comprise co-therapy of a CBSI compound of the invention with other drugs such as anti-hypertensives that comprise a beta blocker such as acebutolol, atenolol, bisoprolol, carvedilol, esmolol, labetalol, metoprolol, nadolol, nebivolol, propranolol, sotalol, timolol, and the like; or a calcium channel blocker such as amlodipine, bepridil, diltiazem, felodipine, nicardipine, nifedipine, nisoldipine, verapamil, and the like. Often either the beta-blocker or the calcium channel blocker is combined with an angiotensin-converting enzyme (ACE) inhibitor or an angiotensin 2 receptor blocker (ARB). The methods of the invention may comprise co-therapy of a CBSI compound of the invention with an angiotensin-converting enzyme (ACE) inhibitor such as ramipril, captopril, enalapril, lisinopril, perindopril, trandolapril, and the like; or an angiotensin 2 receptor blocker (ARB) such as candesartan, losartan, vasartan, and the like. These combinations of anti-hypertensive therapy can be used, if needed, to lower blood pressure, to avoid accelerating the atherosclerotic process, and to relieve the workload on the heart. The methods of the invention may further comprise co-therapy of a CBSI compound of the invention with a thiazide diuretic such as hydrochlorothiazide, chlorthalidone, indapamide, metolazone, bendroflumethiazide, chlorothiazide, and the like.

While some anti-hypertensive agents such as beta-blockers and calcium channel blockers may provide relief from angina (chest pain), other anti-anginal agents include nitroglycerin (widens the heart arteries to reduce chest pain) or ranolazine. The methods of the invention may further comprise co-therapy of a CBSI compound of the invention with a nitroglycerin agent, ranolazine, and the like.

Both SGLT2is and GLP-1 RAs have been shown to reduce the risk of major cardiovascular events (MACE) like myocardial infarction, stroke, and cardiovascular death in clinical trials, making them particularly beneficial for individuals with established ASCVD. Accordingly, these agents are sometimes indicated in ASCVD that is complicated by hyperglycemia or diabetes, heart failure, obesity, and/or renal disease. Classes of anti-diabetic agents most commonly used in ASCVD include glucagon-like peptide-1 receptor agonists (GLP-lRA) and sodium-glucose cotransporter 2 inhibitors (SGLT2i). The methods of the invention may further comprise co-therapy of a CBSI compound of the invention with a glucagon-like peptide-1 receptor agonist (GLP-lRA) such as liraglutide, semaglutide, dulaglutide, albiglutide, and the like; or a sodium-glucose cotransporter 2 inhibitors (SGLT2i) such as empaglifozin, canaglifozin, dapaglifozin, sotaglifozin, and the like.

In overview, typical pharmacotherapy for ASCVD, CAD, or PAD comprises a combination of lipid-lowering agents and anti-platelet agents, possibly further comprising at least one of other anti-coagulants, anti-hypertensives, anti-anginals, and anti-diabetic agents. Given the complexity of possible pharmacotherapy regimens for these diseases, and multiple examples of PGP and/or CYP3A4 inducers, substrates or inhibitors therein including some with a narrow therapeutic index, combinations of colchicine+typical pharmacotherapy creates a significant liability for iatrogenic toxicity and/or drug-drug interactions. For example, many combinations of typical pharmacotherapy and colchicine could result in increased levels of colchicine, a statin, and/or a DOAC, and expose the patients to increased risk of the life-threatening adverse events of these agents, such as cumulative myotoxicity or renal toxicity, and/or excessive bleeding. In contrast, replacing colchicine with a CBSI compound of the invention does not increase the risk of adverse events or drug-drug interaction when combined with these agents or other typical pharmacotherapy in ASCVD, PAD or CAD, as defined herein.

The following examples are presented in order to more fully illustrate the preferred embodiments of the invention. They should in no way, however, be construed as limiting the broad scope of the invention.

### EXAMPLES

The Examples set forth below are for illustrative purposes only and are not intended to limit, in any way, the scope of the present invention.

### Example 1

Phase 2, Randomized, Placebo-Controlled, Efficacy and Safety Study to Slow the Progression or Promote Regression of Coronary Artery Disease (CAD) with CBSI Compounds of the Invention

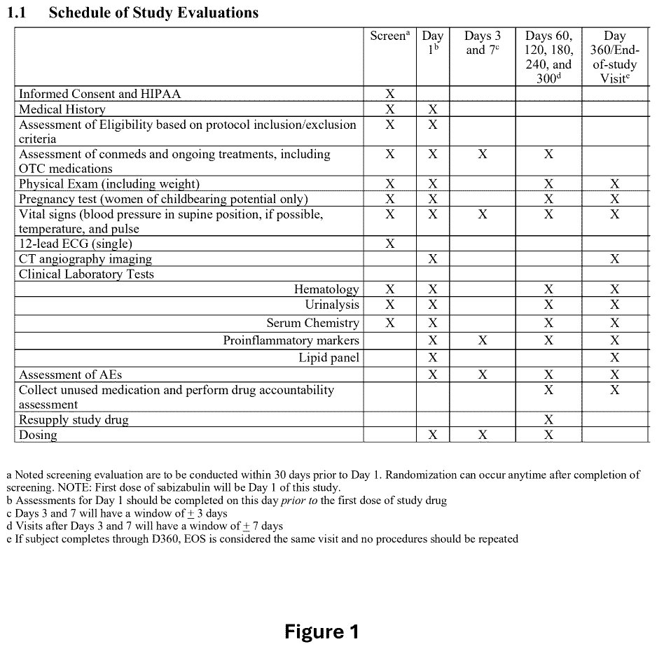

Efficacy of CBSI compounds of the invention will be tested in a Phase 2, double-blind, placebo-controlled, efficacy and safety study of oral compound 17ya, a CBSI compound of this invention, to slow the progression or promote the regression of atherosclerosis in patients with stable CAD. Approximately 120 patients will be randomized in a blinded fashion to three treatment groups (40 patients/arm): Compound 17ya 4.5 mg twice a day, compound 17ya 1.5 mg twice a day, and matching placebo. Patients will have stable coronary artery disease for ≥6 months prior to the date of initiation of screening and will be taking a statin or PCSK-9 inhibitors to manage lipid levels. Patients will receive the study drug for 12 months. The primary endpoint for the study will be percent change from baseline in low attenuation non-calcified plaque volume (LAPV) in the coronary arteries as measured by coronary computed tomography angiography (CCTA). Low attention plaque volume has been shown be an excellent predictor of cardiovascular events in the SCOT-HEART Trial (Scottish Computed Tomography of the HEART) (Williams M C et al. Circulation 141:1452-1462, 2020; Figtree G A et al. Circulation 146:1712-1727, 2022). Key secondary endpoints will be percent change from baseline in non-calcified plaque volume in ischemic arteries, high sensitivity C-reactive protein (CRP) blood levels to assess the contribution of inflammation, and physical function as measured by 6-minute walk test. Further secondary endpoints will be percent change in the following: (a) total atheroma volume, (b) non-calcified plaque volume, (c) dense calcified plaque volume, and (d) remodeling index as measured by CTA; as well as percent change in pericoronary adipose tissue attenuation, epicardial adipose tissue volume and attenuation, brachial flow mediated dilation, and blood values of pro-inflammatory cytokines as biomarkers of inflammation including high sensitivity C-reactive protein (CRP), interferon (INF) γ, ferritin, interleukin (IL)-7, IL-15, IL-18, monocyte chemoattractant protein (MCP) 1, macrophage inflammatory protein (MIP)-1α, and MIP-1β. The key safety assessment will be the proportion of patients that experience a new or worsening cardiovascular event including major adverse cardiovascular events. The trial is summarized in FIG. 1 which shows an expected schedule of study evaluations.

Expected results include that Compound 17ya will demonstrate significant dose-dependent reductions in coronary artery LAPV compared to baseline (promote regression) or compared the placebo group (slow progression). Reductions may be on the order of 40% for Compound 17ya compared to placebo. Similarly, Compound 17ya is expected to slow the progression or promote regression relative to placebo in terms of coronary atherosclerosis (above) but also other atherosclerosis criteria such as total atheroma volume, non-calcified plaque volume, dense calcified plaque volume and remodeling index. Further, Compound 17ya compared to placebo is expected to decrease pericoronary and epicardial adipose tissue volume or attenuation. These reductions may be to levels lower than baseline in the same individual such that treatment is observed to promote regression of CAD (if in coronary artery) or PAD (if in peripheral artery). Alternatively, the reductions may be relatively lower than placebo treated patients but higher than baseline such that treatment is observed to slow progression of CAD or PAD. Lastly, the anti-atherosclerotic observations above are expected to be correlated with reductions in most, if not all, pro-inflammatory biomarkers listed above. E.g., CRP levels in Compound 17ya patients may be reduced by about 40% relative to placebo treated patients.

### Example 2

Phase 2, Randomized, Placebo-Controlled, Efficacy and Safety Study to Slow the Progression or Promote Regression of Peripheral Artery Disease (PAD) with CBSI Compounds of the Invention

Efficacy of CBSI compounds of the invention will be tested in a Phase 2, double-blind, placebo-controlled, efficacy and safety study of oral compound 17ya, a CBSI compound of this invention, to slow the progression or promote the regression of atherosclerosis in patients with stable PAD. Approximately 120 patients will be randomized in a blinded fashion to three treatment groups (40 patients/arm): Compound 17ya 4.5 mg twice a day, compound 17ya 1.5 mg twice a day, and matching placebo. Patients will have stable peripheral artery disease (PAD) for ≥6 months prior to the date of initiation of screening and will be taking a statin or PCSK-9 inhibitors to manage lipid levels. Patients will receive the study drug for 12 months. The primary endpoint for the study will be percent change from baseline in low attenuation non-calcified plaque volume (LAPV) in the peripheral arteries as measured by computed tomography angiography (CTA). Low attention plaque volume has been shown be an excellent predictor of cardiovascular events in the SCOT-HEART Trial (Scottish Computed Tomography of the HEART) (Williams M C et al. Circulation 141:1452-1462, 2020; Figtree G A et al. Circulation 146:1712-1727, 2022). Key secondary endpoints will be percent change from baseline in non-calcified plaque volume in ischemic arteries, high sensitivity C-reactive protein (CRP) blood levels to assess the contribution of inflammation, and physical function as measured by 6-minute walk test. Further secondary endpoints will be percent change in the following: (a) total atheroma volume, (b) non-calcified plaque volume, (c) dense calcified plaque volume, and (d) remodeling index as measured by CTA; as well as percent change in pericoronary adipose tissue attenuation, epicardial adipose tissue volume and attenuation, brachial flow mediated dilation, and blood values of pro-inflammatory cytokines as biomarkers of inflammation including high sensitivity C-reactive protein (CRP), interferon (INF) γ, ferritin, interleukin (IL)-7, IL-15, IL-18, monocyte chemoattractant protein (MCP) 1, macrophage inflammatory protein (MIP)-1α, and MIP-1β. The key safety assessment will be the proportion of patients that experience a new or worsening peripheral vascular or cardiovascular event including major adverse cardiovascular events. The trial is summarized in FIG. 1 which shows an expected schedule of study evaluations.

Expected results are that Compound 17ya will demonstrate significant dose-dependent reductions in specified peripheral artery LAPV compared to baseline (promote regression) or compared the placebo group (slow progression). Reductions may be on the order of 40% for Compound 17ya compared to placebo. Similarly, Compound 17ya is expected to slow the progression or promote regression relative to placebo in terms of peripheral or coronary atherosclerosis but also other atherosclerosis criteria such as total atheroma volume, non-calcified plaque volume, dense calcified plaque volume and remodeling index. Further, Compound 17ya compared to placebo is expected to decrease pericoronary and epicardial adipose tissue volume or attenuation. These reductions may be to levels lower than baseline in the same individual such that treatment is observed to promote regression of CAD (if in coronary artery) or PAD (if in peripheral artery). Alternatively, the reductions may be relatively lower than placebo treated control but higher than baseline such that treatment is observed to slow progression of CAD or PAD. Lastly, the anti-atherosclerotic observations above are expected to be correlated with reductions in most, if not all, pro-inflammatory biomarkers listed above. E.g., CRP levels in Compound 17ya patients may be reduced by about 40% relative to placebo treated patients.

### Example 3

Collagen Antibody-Induced Rheumatoid Arthritis (CAIA) Efficacy Model in Mice Treated with CBSI Compounds of the Invention

Inflammatory arthritis affects millions of humans around the world each year and the treatments currently available are limited to reducing disease symptoms, but do not modify the disease state. Animal models of arthritis mimic many of the features of arthritis in humans and have been used successfully in establishing proof of concept for new treatment compounds. Of the animal models available, the murine collagen antibody-induced arthritis model appears to be relevant and is widely used because of the similar putative etiology to human disease. The mouse collagen antibody induced arthritis (CAIA) model is induced by a primary injection of collagen antibodies (inducing agent) followed by a boost immunization with LPS (boost agent). The boost injection is given on Day 6 and erosive polyarthritis usually becomes evident starting Day 7. The objective of the present study is to evaluate the efficacy of the Compound 17ya in the collagen-antibody induced arthritic (CAIA) mouse model in Balb/c mice (females, 8-9 weeks old from Charles River Laboratories). The inducing agent for this experiment will be ArthritoMab (an arthritogenic monoclonal antibody available from MD Bioproducts), whereas the boost agent will be lipopolysaccharides cocktail (LPS) also from MD Bioproducts. The test article will be Compound 17ya which will be compared to a positive control agent, dexamethasone.

Ten animals have been assigned to five treatment groups (fifty total animals) that include: (1) Non-disease (no ArthritoMab) plus vehicle, (2) ArithritoMab/LPS plus vehicle, (3) ArithritoMab/LPS plus Compound 17ya (2 mg/kg), (4) ArithritoMab/LPS plus Compound 17ya (4 mg/kg), and (5) ArithritoMab/LPS plus dexamethasone (0.3 mg/kg). The ArthritoMab was given (0.2 mL of the 10 mg/mL inducing agents as supplied) to groups 2-5 on Day 1 via IV bolus injections. The LPS was reconstituted in phosphate buffered saline at pH 7.4 to a final concentration of 0.5 mg/mL which is administered on Day 6 to groups 2-5 as a 0.1 mL (50 μg) IP injection. Compound 17ya (groups 3 and 4), dexamethasone (group 5) or vehicle (groups 1 and 2) is administered daily starting at Day 7 until Day 20 via oral gavage.

In Life Assessments: Animals will be checked on Days 1-20 for mortality or moribundity, and body weight. Clinical observations will be made related to arthritis model such as swelling and redness of paws, and will be used for disease scoring evaluation (once pre-treatment, and on Days 7 and 8, then 3 times a week during treatment). Paw volume of the hindlimbs will be measured using a plethysmometer once pre-treatment and on Days 7 and 8 then 3 time a week during the treatment period. The sum of volume will be calculated. In brief, the Plethysmometer is a volume meter, designed for accurate measurements of inflammation-induced swelling. It consists of a water filled cell into which the paw along with the ankle joint is dipped. A transducer records the differences in water level caused by volume displacement and provides LCD readout of the exact volume gain due to swelling. Clinical score of the 4 limbs will be evaluated as per below:

### Clinical Disease Evaluation Score (0 to 4)

| | |
|---|---|
| 0 | Normal |
| 1 | Erythema and mild swelling confined to the mid-foot (tarsals) or ankle, or digits. |
| 2 | Erythema and mild swelling extended to 2 segments of the paw OR erythema and moderate |
| | swelling confined from 1 to 2 segments of the paw. |
| 3 | Erythema and moderate swelling extended to mid foot (tarsal) + ankle + digits. |
| 4 | Erythema and severe swelling extended to mid foot (tarsal) + ankle + digits. The paw is shiny. |

Euthanasia: On Day 21 at the end of the final in-life assessment, all mice from Groups 1 to 5 will be anesthetized by deep isoflurane anesthesia and exsanguination of the abdominal aorta. All 4 limbs will be collected from all animals, and the skin removed, and snap frozen in liquid nitrogen and stored at −80° C. for cytokine analysis. The left hindlimb (without skin) will be fixed in 10% neutral buffered formalin (NBF) for histopathology analysis. The forelimbs will be snap frozen and stored in a freezer set to maintain −80° C. for possible future analysis. Histology: The left hindpaws from all animals will be decalcified, embedded in paraffin, sectioned, mounted on glass slides and stained with hematoxylin and eosin (H&E). Microscopic Evaluation: The slides will be evaluated by a veterinary pathologist using a semi-quantitative scoring scale for inflammation, pannus/synovial hyperplasia, cartilage damage, bone resorption and periosteal/exostotic changes. Data will be reported in an Excel spreadsheet. Cytokine Analysis: The right hindlimbs from all animals will be analyzed by CRL-SEN Biomarkers group for TNFα, IL-6, IL-10, IL-1β and IFN-γ using a bead-based multiplexed assay. In life-observations are ongoing but not complete, and euthanasia is expected within a month, with a final report soon thereafter.

Expected results include significant signs and symptoms of CAIA disease observed in group 2 (inducer/booster plus vehicle) compared to group 1 (no inducers/no booster), and dose-dependent attenuation of CAIA disease in groups 3 and 4 (2 and 4 mg/kg Compound 17ya) that is comparable to group 5 (dexamethasone as positive control). Expected in-life observations are significantly increased swelling and redness of paws, and increased paw volume in group 2 vs 1, and significantly reduced values in groups 3-5 compared to group 2, possibly reduced to as low as group 1. Expect Compound 17ya to demonstrate efficacy similar to or possibly in excess of dexamethasone. This would indicate that treatment with 2 mg/kg 17ya and/or 4 mg/kg 17ya and/or dexamethasone, slowed progression or promoted regression of the CAIA disease state based on in-life clinical observations. Expected observations in post-euthanasia mice include significantly increased signs/symptoms of CAIA disease in group 2 vs 1, and significant reductions in groups 3-5 vs 2. E.g., histology slides may reveal inflammation and hyperplasia, cartilage damage, bone resorption and periosteal/exostotic changes in group 2 that are prevented or attenuated in groups 3-5. Further, the pro-inflammatory cytokines analysis should reveal pan-elevated cytokine levels in group 2 vs 1, and significant reductions thereof in groups 3-5, possibly reducing to baseline or non-disease (group 1) levels. Again, expect Compound 17ya to show efficacy across the cytokine panel that is similar to in excess of dexamethasone.

All of the features described herein (including any accompanying claims, abstract and drawings), and/or all of the steps of any method or process so disclosed, may be combined with any of the above aspects in any combination, except combinations where at least some of such features and/or steps are mutually exclusive. Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of treating, reducing, inhibiting, slowing the progression of, promoting the regression of, or preventing atherosclerotic cardiovascular disease (ASCVD) in a subject in need thereof by administering to the subject a formulation having a therapeutically effective amount of a colchicine binding site inhibitor (CBSI) compound of Formula (I): 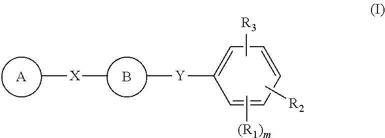
wherein
A is phenyl, indolyl, or indazolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole or benzimidazole, optionally substituted with at least one of (C₁-C₄)alkyl, halo(C₁-C₄)alkyl, O—(C₁-C₄)alkyl, O-halo(C₁-C₄)alkyl, F, Cl, Br, I, CN, —CH₂CN, hydroxyl, or NO₂;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond or NH;
Y is —C═O; and
m is 1-3, or a pharmaceutically acceptable salt, hydrate, polymorph, or isomer thereof.

2. The method of treating, reducing, inhibiting, slowing the progression of, promoting the regression of, or preventing ASCVD according to claim 1, wherein A is indolyl, optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
B is an imidazole, optionally substituted with at least one of (C₁-C₄)alkyl;
R₁, R₂ and R₃ are independently at least one of hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂;
X is a bond;
Y is —C═O; and
m is 1-3, or a pharmaceutically acceptable salt, hydrate, polymorph, or isomer thereof.

3. The method of treating, reducing, inhibiting, slowing the progression of, promoting the regression of, or preventing ASCVD according to claim 1, wherein said compound of Formula I is represented by a compound of the Formula VII: 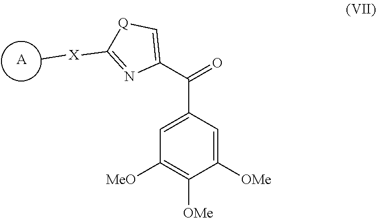
wherein
X is a bond or NH;
Q is NH and
A is a phenyl, indolyl, or indazolyl ring optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer thereof.

4. The method according to claim 3, wherein X is a bond.

5. The method according to claim 3, wherein X is a bond; Q is NH; and A is an indolyl ring optionally substituted with at least one of (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; or a pharmaceutically acceptable salt, hydrate, polymorph, or isomer thereof.

6. A method of treating, reducing, inhibiting, slowing the progression of, promoting the regression of, or preventing ASCVD in a subject in need thereof by administering to the subject a formulation having a therapeutically effective amount of a colchicine binding site inhibitor (CBSI) compound of the Formula VII(c): 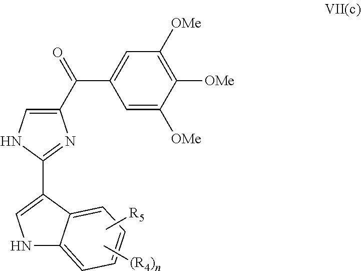
wherein
R₄ and R₅ are independently hydrogen, (C₁-C₄)alkyl, halo(C₁-C₄)alkyl, O—(C₁-C₄)alkyl, O—(C₁-C₄)haloalkyl, (C₁-C₄)alkylamino, amino(C₁-C₄)alkyl, F, Cl, Br, I, CN, —CH₂CN, NH₂, hydroxyl, OC(O)CF₃, —OCH₂Ph, —NHCO—(C₁-C₄)alkyl, COOH, —C(O)Ph, C(O)O—(C₁-C₄)alkyl, C(O)H, —C(O)NH₂ or NO₂; and
n is 1-4; or a pharmaceutically acceptable salt, hydrate, polymorph, or isomer thereof.

7. A method of treating, reducing, inhibiting, slowing the progression of, promoting the regression of, or preventing ASCVD in a subject in need thereof by administering to the subject a formulation having a therapeutically effective amount of a colchicine binding site inhibitor (CBSI) compound 17ya represented as:

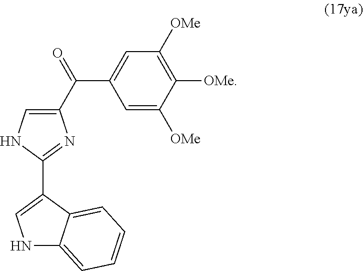 [SMILES: COc1cc(C(=O)c2c[nH]c(-c3c[nH]c4ccccc34)n2)cc(OC)c1OC]

8. The method of treating, reducing, inhibiting, slowing the progression of, promoting the regression of, or preventing ASCVD according claim 1, wherein said ASCVD is at least one of a cerebrovascular accident (CVA), transient ischemic accident (TIA), er abdominal aortic aneurysm (AAA), coronary artery disease (CAD), or peripheral artery disease (PAD).

9. (canceled)

10. The method of claim 8, wherein said CAD is stable CAD.

11. The method of claim 8, wherein said administering of said CBSI compound treats, reduces, inhibits, slows the progression of, promotes the regression of, or prevents intermittent claudication, critical limb ischemia, or atherothrombosis of the heart, brain or limbs.

12. The method of either claim 1, wherein said administering of said CBSI compound any one of slows the progression, promotes the regression of atheromatous plaques, improves atheromatous plaque morphology or atheromatous plaque composition.

13. The method of either claim 1, wherein said administering of said CBSI compound treats, reduces, inhibits, slows the progression of, promotes the regression of, or prevents at least one of the following: plaque initiation, plaque progression, plaque rupture and/or plaque related thrombosis and its complications.

14. (canceled)

15. The method according to claim 12, wherein said administering of said CBSI compound improves plaque morphology or plaque composition by reducing plaque volume, decreasing plaque necrotic core size, increasing fibrous cap thickness of the plaque, decreasing the extent of calcification of the plaque, and/or decreasing maladaptive plaque remodeling.

16. The method according to claim 15, wherein said administering of said CBSI compound decreases the low-attenuation plaque volume (LAPV) in said subject measured by computed tomography angiography (CTA) or coronary computed tomography angiography (CCTA).

17. The method of claim 1, wherein said administering of said CBSI compound decreases blood levels of proinflammatory cytokines.

18. The method of claim 17, wherein said proinflammatory cytokine is one or more of high sensitivity C-reactive protein (CRP), interferon (INF) γ, ferritin, interleukin (IL)-7, IL-15, IL-18, monocyte chemoattractant protein (MCP) 1, macrophage inflammatory protein (MIP)-1α, or MIP-1β.

19. The method according claim 1, wherein said administering of said CBSI compound decreases both LAPV and CRP.

20. The method of claim 1, wherein said administering of said CBSI compound treats, inhibits, reduces the incidence of, or prevents adverse cardiovascular events or acute coronary syndrome (ACS) that occur secondary to at least one of said ASCVD, CAD, or PAD.

21. (canceled)

22. The method of claim 1, wherein said administering of said CBSI compound treats, inhibits, reduces the incidence of, or prevents at least one of acute myocardial infarction (AMI), unstable angina (USA), chest pain or pressure, shortness of breath, excessive sweating, dizziness, or fatigue.

23. The method of claim 1, wherein said administering of said CBSI compound treats, inhibits, reduces the incidence of, or prevents major adverse cardiovascular events (MACE).

24. The method of claim 23, wherein said MACE events include but are not limited to myocardial infarction, stroke, death, heart failure, hospitalization for heart failure, revascularization, e.g., percutaneous coronary intervention (PCI) or coronary artery bypass graft, arrhythmias, endocarditis, aortic dissection, or thrombotic events.

25. The method of claim 23, wherein said administering of said compound treats, inhibits, reduces the incidence of, or prevents MACE despite ongoing ACS or uncontrolled risk factors.

26. The method of claim 25, wherein said uncontrolled risk factors include but are not limited to age, diabetes mellitus, hypertension, urologic conditions, chronic kidney disease, smoking, high LDL cholesterol, low HDL cholesterol, elevated triglycerides, family history of CAD, obesity, physical inactivity, or a history of previous cardiovascular events.

27. The method of claim 1, wherein said administering of said CBSI compound slows the progression of or reverses the decline of physical function.

28. (canceled)

29. The method of claim 1, wherein said CBSI compound provides the therapeutic benefit of colchicine without the risk of the adverse events associated with colchicine.

30. The method of claim 29, wherein at least one of ASCVD, CAD, or PAD is treated with reduced risk of, or without the risk of, adverse events associated with colchicine; said adverse events including but not limited to diarrhea, vomiting, abdominal cramps, neutropenia, blood dyscrasias, myalgia, rhabdomyolysis, or neurotoxicity.

31. The method of claim 29, wherein said CBSI compound is administered in combination with other drugs used for at least one of said ASCVD, CAD, or PAD.

32. The method of claim 31, wherein said other drugs comprise at least one of lipid-lowering agents, anti-platelet agents, anti-coagulants, anti-hypertensives, anti-anginal agents, or anti-diabetics.

33. (canceled)

34. The method of claim 31, wherein said combination can be administered with reduced risk of, or without the risk of, drug-drug interactions (DDI) between said CBSI compound and said other drugs used for ASCVD, CAD, or PAD.

35. The method of claim 34, wherein said other drugs used for ASCVD, CAD, or PAD include an HMG-CoA reductase inhibitor (statin).

36. The method of claim 35, wherein said statin is at least one of atorvastatin, fluvastatin, lovastatin, lovastatin extended-release, pitavastatin, pravastatin, rosuvastatin, or simvastatin.

37. The method of claim 29, wherein said CBSI compound is co-administered with at least one of an inhibitor of CYP3A4 or an inhibitor of PGP.

38. The method of claim 37, wherein said CYP3A4 or PGP inhibitor is at least one of a direct acting oral anticoagulant, amiodarone, diltiazem, grapefruit juice, atorvastatin, lovastatin, ranolazine, simvastatin, or verapamil.

39. The method of claim 29, wherein said subject has renal or hepatic impairment.

40. The method of claim 1, wherein said CBSI compound can be administered with reduced risk of, or without the risk of, of drug toxicity despite administration of accepted therapeutic doses, or without need for dose adjustments.

41. (canceled)

42. The method of claim 1, wherein said CBSI compound is administered with other drugs used in ASCVD despite a history of intolerable adverse events due to colchicine therapy in the subject.

43. The method of treating said ASCVD according to claim 1, wherein the CBSI compound is administered in an amount of about 0.1 to about 100 mg twice daily, or in an amount of about 1 mg to about 10 mg twice daily, or in an amount of about 1.5 mg to 4.5 mg twice daily.

44-45. (canceled)

46. The method of treating said ASCVD according to claim 1, further comprising a pharmaceutically acceptable excipient.
